SEQUENCE LISTING (103 sequences)

SEQ ID NO: 1 - protein, 217 aa, homo sapiens
CEVQLVESGGGLVKPGGSLRLSCAASGFTFRSYSMNWVRQAPGKGLEWVSSISSSGNYIYYADSVKGRFTLSRDNAKNSLYLQMNSLRAEDTAVYYCARGGGTSWSHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKSE

SEQ ID NO: 2 - protein, 220 aa, homo sapiens
VVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDAYDSSGPDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 3 - protein, 225 aa, homo sapiens
AVQLVQSGAEVKKPGESLRISCKGSGYSFTSYWISWVRQMPGKGLEWMGRIDPSDSYTNYSPSFQGHVTISADKSISTAYLQWSSLKTSDTAMYYCARPLQTYSIASVGHWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLP

SEQ ID NO: 4 - protein, 219 aa, homo sapiens
QITLKESGPTLVKPTQTLTLTCTFSGFSLNTPGVGVGWIRQPPGKALEWLALIYWDDDKRYRPSLESRLTITKDTSKNHVVLTMTNMDPVDTATYFCAHKNLQYSEWFDPWGQGTLVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 5 - protein, 217 aa, homo sapiens
PVQLQESGPGLVKPSETLSLTCTVSGGSMSIRSSYWGWIRQSPGKGLEWIGHIFYSGSTYYNPSLQSRVTILVDTSKNQFSLRLSSVTAADTAVYYCVRSFGVARWDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 6 - protein, 217 aa, homo sapiens
QFMLTQPHSVSESPGKTVTISCTRSSGSIASNYVQWYQQRPGSAPTTVIYEDNERPSGVPDRFSGSIDSSSNSASLTISGLKTEDEADYYCQSYDSNNRWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 7 - protein, 216 aa, homo sapiens
ASALTQPASVSGSPGQSITISCTGTSSDVGDYNYVSWYQQHPGKAPKLMIYDVSNRPSGVSNRFSGSKSGNTASLTISGLQAEDEADYYCSSYTSSSTLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 217 aa, homo sapiens
ASALTQPASVSGSPGQSITISCTGTSSDVGSYNLVSWYQQHPGKAPKLMIYEGSKRPSGVSNRFSGSKSGNTASLTISGLQAEDEADYYCCSYAGSSTFAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 215 aa, homo sapiens
EMVLTQSPATLSLSPGERATLSCRASQSVSRNSLAWYQQRPGQTPRLLIYGASSRATGIPDRFSGSGSGTDFTLIISRLEPEDFAVYFCLQYDESPYTFGQGAKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 10 - protein, 215 aa, homo sapiens
DIQMTQSPSSLSASVGDRVTITCQASQNIRNYLNWYQQKPGKAPKLLIYDASNLETGVPSRFSGSGSGTDFTLTISSLQPEDIATYYCQQYDNLLLFTFGPGTTVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 11 aa, homo sapiens
ARGGGTSWSHY

SEQ ID NO: 12 - protein, 15 aa, homo sapiens
AKDAYDSSGPDAFDI

SEQ ID NO: 13 - protein, 14 aa, homo sapiens
ARPLQTYSIASVGH

SEQ ID NO: 14 - protein, 13 aa, homo sapiens
AHKNLQYSEWFDP

SEQ ID NO: 15 - protein, 11 aa, homo sapiens
VRSFGVARWDF

SEQ ID NO: 16 - protein, 10 aa, homo sapiens
QSYDSNNRWV

SEQ ID NO: 17 - protein, 10 aa, homo sapiens
SSYTSSSTLV

SEQ ID NO: 18 - protein, 11 aa, homo sapiens
CSYAGSSTFAV

SEQ ID NO: 19 - protein, 9 aa, homo sapiens
LQYDESPYT

SEQ ID NO: 20 - protein, 10 aa, homo sapiens
QQYDNLLLFT

SEQ ID NO: 21 - protein, 8 aa, homo sapiens
ISSSGNYI

SEQ ID NO: 22 - protein, 8 aa, homo sapiens
ISGSGGST

SEQ ID NO: 23 - protein, 8 aa, homo sapiens
IDPSDSYT

SEQ ID NO: 24 - protein, 7 aa, homo sapiens
IYWDDDK

SEQ ID NO: 25 - protein, 7 aa, homo sapiens
IFYSGST

SEQ ID NO: 26 - protein, 3 aa, homo sapiens
EDN

SEQ ID NO: 27 - protein, 3 aa, homo sapiens
DVS

SEQ ID NO: 28 - protein, 3 aa, homo sapiens
EGS

SEQ ID NO: 29 - protein, 3 aa, homo sapiens
GAS

SEQ ID NO: 30 - protein, 3 aa, homo sapiens
DAS

SEQ ID NO: 31 - protein, 8 aa, homo sapiens
GFTFRSYS

SEQ ID NO: 32 - protein, 8 aa, homo sapiens
GFTFSSYA

SEQ ID NO: 33 - protein, 8 aa, homo sapiens
GYSFTSYW

SEQ ID NO: 34 - protein, 10 aa, homo sapiens
GFSLNTPGVG

SEQ ID NO: 35 - protein, 10 aa, homo sapiens
GGSMSIRSSY

SEQ ID NO: 36 - protein, 8 aa, homo sapiens
SGSIASNY

SEQ ID NO: 37 - protein, 9 aa, homo sapiens
SSDVGDYNY

SEQ ID NO: 38 - protein, 9 aa, homo sapiens
SSDVGSYNL

SEQ ID NO: 39 - protein, 7 aa, homo sapiens
QSVSRNS

SEQ ID NO: 40 - protein, 6 aa, homo sapiens
QNIRNY

SEQ ID NO: 41 - DNA, 652 bp, homo sapiens
gtgtgaggtgcagctggtggagtctgggggaggcctggtcaagcctggggggtccctgagactctcctgtgcagcctctggattcaccttcaggagctatagcatgaactgggtccgccaggctccagggaaggggctggagtgggtctcatccattagtagtagtggtaattacatatactacgcagactcagtgaagggccgattcaccctctccagagacaacgccaagaactcactgtatctgcaaatgaacagcctgagagccgaggacacggctgtgtattactgtgcgagaggtgggggtaccagctggtcgcattactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagtgag

SEQ ID NO: 42 - DNA, 660 bp, homo sapiens
gtggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatgcatatgatagtagtggcccagatgcttttgatatctggggccaagggacaatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagtg

SEQ ID NO: 43 - DNA, 675 bp, homo sapiens
gccgtgcagctggtgcagtccggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctacagtggagcagcctgaagacctcggacaccgccatgtattactgtgcgagaccgctacaaacttatagtatagcatcagtaggacactggggccagggaaccctggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccg

SEQ ID NO: 44 - DNA, 659 bp, homo sapiens
cccagatcaccttgaaggagtctggtccgacgctggtgaagcccacacagaccctcacgctgacctgcaccttctctgggttctcactcaacactcctggagtgggtgtgggctggatccgtcagcccccaggaaaggccctggaatggcttgcactcatttattgggatgatgataagcgctacaggccatctctggagagcaggctcaccatcaccaaggacacctccaaaaaccacgttgtccttacgatgaccaacatggaccctgtggacacagccacatatttttgtgcacacaagaaccttcagtattcggaatggttcgacccctggggccagggcaccctggtcattgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaatgtagatcacaagcccagcaacaccaaggtggacaagagagtt

SEQ ID NO: 45 - DNA, 651 bp, homo sapiens
cccgtgcagctgcaggagtcgggcccaggactggtgaagccttcggagaccctgtccctcacctgcactgtctctggtggctccatgagcattaggagttcctactggggctggatccgccagtcaccagggaaggggctggagtggattgggcatatattttatagtgggagcacctactacaacccgtccctccagagtcgagtcacaatattagtagacacgtccaagaaccaattctccctgaggctgagctctgtgaccgcagcggacacggccgtgtattactgtgtgagaagttttggcgtggctcgatgggacttctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaggtg

SEQ ID NO: 46 - DNA, 651 bp, homo sapiens
caatttatgctgactcagccccactctgtgtcggagtctccggggaagacggtaaccatctcctgcacccgcagcagtggcagcattgccagcaactatgtgcagtggtaccagcagcgcccgggcagtgcccccaccactgtgatctatgaggataacgaaagaccctctggggtccctgatcggttctctggctccatcgacagctcctccaactctgcctccctcaccatctctggactgaagactgaggacgaggctgactactactgtcagtcttatgatagcaacaatcgttgggtgttcggcggagggaccaagctgaccgtcctaggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 47 - DNA, 648 bp, homo sapiens
gcctctgccctgactcagcctgcctccgtgtctgggtctcctggacagtcgatcaccatctcctgcactggaaccagcagtgacgttggtgattataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcactcttgtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 48 - DNA, 651 bp, homo sapiens
gcctctgccctgactcagcctgcctccgtgtctgggtctcctggacagtcgatcaccatctcctgcactggaaccagcagtgatgttgggagttataaccttgtctcctggtaccaacagcacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgctgctcatatgcaggtagtagcactttcgcggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 49 - DNA, 645 bp, homo sapiens
gaaatggtgttgacgcagtctccagccaccctgtctttgtctccaggggaaagagccaccctctcctgcagggccagtcagagtgttagcagaaactccttagcctggtaccagcagagacctggccagactcccaggctcctcatctatggtgcctccagcagggccactggcatcccagacaggttcagtggcagtgggtctgggacagacttcactctcatcatcagcagactggagcctgaagattttgcagtgtatttctgtctccagtatgatgagtcaccgtacacttttggccagggggccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 50 - DNA, 647 bp, homo sapiens
gtgacatccagatgacccagtctccatcctccctgtctgcatctgtaggagacagagtcaccatcacttgccaggcgagtcagaacattaggaattatttaaattggtatcagcagaaaccagggaaagcccctaagctcctgatctacgatgcatccaatttggaaacaggggtcccatcaaggttcagtggaagtggatctgggacagattttactctcaccatcagcagcctgcagcctgaagatattgcaacatattactgtcaacagtatgataatctcctcctattcactttcggccctgggaccacagttgatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 51 - DNA, 33 bp, homo sapiens
gcgagaggtgggggtaccagctggtcgcattac

SEQ ID NO: 52 - DNA, 45 bp, homo sapiens
gcgaaagatgcatatgatagtagtggcccagatgcttttgatatc

SEQ ID NO: 53 - DNA, 42 bp, homo sapiens
gcgagaccgctacaaacttatagtatagcatcagtaggacac

SEQ ID NO: 54 - DNA, 39 bp, homo sapiens
gcacacaagaaccttcagtattcggaatggttcgacccc

SEQ ID NO: 55 - DNA, 33 bp, homo sapiens
gtgagaagttttggcgtggctcgatgggacttc

SEQ ID NO: 56 - DNA, 30 bp, homo sapiens
cagtcttatgatagcaacaatcgttgggtg

SEQ ID NO: 57 - DNA, 30 bp, homo sapiens
agctcatatacaagcagcagcactcttgta

SEQ ID NO: 58 - DNA, 33 bp, homo sapiens
tgctcatatgcaggtagtagcactttcgcggta

SEQ ID NO: 59 - DNA, 27 bp, homo sapiens
ctccagtatgatgagtcaccgtacact

SEQ ID NO: 60 - DNA, 30 bp, homo sapiens
caacagtatgataatctcctcctattcact

SEQ ID NO: 61 - DNA, 24 bp, homo sapiens
attagtagtagtggtaattacata

SEQ ID NO: 62 - DNA, 24 bp, homo sapiens
attagtggtagtggtggtagcaca

SEQ ID NO: 63 - DNA, 24 bp, homo sapiens
attgatcctagtgactcttatacc

SEQ ID NO: 64 - DNA, 21 bp, homo sapiens
atttattgggatgatgataag

SEQ ID NO: 65 - DNA, 21 bp, homo sapiens
atattttatagtgggagcacc

SEQ ID NO: 66 - DNA, 9 bp, homo sapiens
gaggataac

SEQ ID NO: 67 - DNA, 9 bp, homo sapiens
gatgtcagt

SEQ ID NO: 68 - DNA, 9 bp, homo sapiens
gagggcagt

SEQ ID NO: 69 - DNA, 9 bp, homo sapiens
ggtgcctcc

SEQ ID NO: 70 - DNA, 9 bp, homo sapiens
gatgcatcc

SEQ ID NO: 71 - DNA, 24 bp, homo sapiens
ggattcaccttcaggagctatagc

SEQ ID NO: 72 - DNA, 24 bp, homo sapiens
ggattcacctttagcagctatgcc

SEQ ID NO: 73 - DNA, 24 bp, homo sapiens
ggatacagctttaccagctactgg

SEQ ID NO: 74 - DNA, 30 bp, homo sapiens
gggttctcactcaacactcctggagtgggt

SEQ ID NO: 75 - DNA, 30 bp, homo sapiens
ggtggctccatgagcattaggagttcctac

SEQ ID NO: 76 - DNA, 24 bp, homo sapiens
agtggcagcattgccagcaactat

SEQ ID NO: 77 - DNA, 27 bp, homo sapiens
agcagtgacgttggtgattataactat

SEQ ID NO: 78 - DNA, 27 bp, homo sapiens
agcagtgatgttgggagttataacctt

SEQ ID NO: 79 - DNA, 21 bp, homo sapiens
cagagtgttagcagaaactcc

SEQ ID NO: 80 - DNA, 18 bp, homo sapiens
cagaacattaggaattat

SEQ ID NO: 81 - protein, 223 aa, homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAREGYCSSTSCYATTLTTGAREPWSPSPQASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 82 - protein, 217 aa, homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEYYYDSSGYYYCFYLGPRDNGHRLFRPPPRAHRSSPWHPPPRAPLGAQRPWAAWSRTTSPNRRCRGTQAPPAACTPSRLSYSPQDSTPSAAWPCPPAAWAPRPTSATITSPATPRWTRK

SEQ ID NO: 83 - protein, 225 aa, homo sapiens
EVQLVQSGAEVKKPGESLRISCKGSGYSFTSYWISWVRQMPGKGLEWMGRIDPSDSYTNYSPSFQGHVTISADKSISTAYLQWSSLKASDTAMYYCARGYSSSWYTTLTTGAREPWSPSPQGSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLP

SEQ ID NO: 84 - protein, 221 aa, homo sapiens
QITLKESGPTLVKPTQTLTLTCTFSGFSLSTSGVGVGWIRQPPGKALEWLALIYWDDDKRYSPSLKSRLTITKDTSKNQVVLTMTNMDPVDTATYYCAHRRYNRNHTTGSTPGAREPWSPSPQASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 85 - protein, 224 aa, homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAREYYDFWSGYYTTTLTTGAREPWSPSPQASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 86 - protein, 210 aa, homo sapiens
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNHWVFGGGTKLTVLAQGCPLGHSVPTLLGASSQQGHTGVSHKLLPGSRDSCLEGRQPRQGGGGDHHTLQTKQQQVRGQQLPEPDAAVEVPQKLQLPGHAREHRGEDSCPYGMF

SEQ ID NO: 87 - protein, 213 aa, homo sapiens
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTLCGIRRRDQADRPRVSPRLPPRSLCSRPPLRSFKPTRPHWCVSVTSTREPQWLGKQIAAPSRREWRPPHPPNKATTSTRPAAIARLSSGSPTEATAARSRMKGAPWRRQWPLQNV

SEQ ID NO: 88 - protein, 211 aa, homo sapiens
QSALTQPASVSGSPGQSITISCTGTSSDVGSYNLVSWYQQHPGKAPKLMIYEGSKRPSGVSNRFSGSKSGNTASLTISGLQAEDEADYYCCSYAGSSTFCGIRRRDQADRPSPRLPPRSLCSHPPLRSFKPTRPHWCVSVTSTREPQLPGRQIAAPSRRGWRPPHPPNKATTSTRPAATARLSSGSPTKATAARSRMKGAPWRRQLPLRNV

SEQ ID NO: 89 - protein, 211 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPVHFWPGDQAGDQTELWLHHLSSSSRHLMSSNLELPLLCACITSIPERPKYSGRWITPSNRVTPRRVSQSRTARTAPTASAAPRAKQTTRNTKSTPAKSPIRAARPSQRASTGES

SEQ ID NO: 90 - protein, 210 aa, homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPPFTFGPGTKVDIKPNCGCTICLHLPAIAVEIWNCLCCVPAELLSQRGQSTVEGGRPPIGLPGECHRAGQQGQHLQPQQHPDAEQSRLRETQSLRLRSHPSGPELARHKELQQGRV

SEQ ID NO: 91 - DNA, 669 bp, homo sapiens
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagaaggatattgtagtagtaccagctgctatgccactactttgactactggggccagggaaccctggtcaccgtctcctcaggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtt

SEQ ID NO: 92 - DNA, 671 bp, homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagagtattactatgatagtagtggttattactactgatgcttttgatatctggggccaagggacaatggtcaccgtctcttcaggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtt

SEQ ID NO: 93 - DNA, 675 bp, homo sapiens
gaagtgcagctggtgcagtccggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcagcctgaaggcctcggacaccgccatgtattactgtgcgagagggtatagcagcagctggtacactactttgactactggggccagggaaccctggtcaccgtctcctcaggggagtgcatccgccccaacccttttccccctcgtctcctgtgagaattccccgtcggatacgagcagcgtggccgttggctgcctcgcacaggacttccttcccgactccatcactttgtcctggaaatacaagaacaactctgacatcagcagtacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttcca

SEQ ID NO: 94 - DNA, 663 bp, homo sapiens
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattgggatgatgataagcgctacagcccatctctgaagagcaggctcaccatcaccaaggacacctccaaaaaccaggtggtccttacaatgaccaacatggaccctgtggacacagccacatattactgtgcacacagacggtataaccggaaccacacaactggttcgacccctggggccagggaaccctggtcaccgtctcctcaggcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagtt

SEQ ID NO: 95 - DNA, 672 bp, homo sapiens
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatcagtagacacgtccaagaaccagttctccctgaagctgagctctgtgaccgccgcggacacggccgtgtattactgtgcgagagagtattacgatttttggagtggttattataccactactttgactactggggccagggaaccctggtcaccgtctcctcaggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtt

SEQ ID NO: 96 - DNA, 646 bp, homo sapiens
aattttatgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaatcattgggtgttcggcggagggaccaagctgaccgtcctagcccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 97 - DNA, 653 bp, homo sapiens
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcagcagcactctctgtggtattcggcggagggaccaagctgaccgtcctagggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 98 - DNA, 647 bp, homo sapiens
cagtctgccctgactcagcctgcctccgtgtctgggtctcctggacagtcgatcaccatctcctgcactggaaccagcagtgatgttgggagttataaccttgtctcctggtaccaacagcacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgctgctcatatgcaggtagtagcactttctgtggtattcggcggagggaccaagctgaccgtcctagcccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 99 - DNA, 650 bp, homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcctgtacacttttggccaggggaccaagctggagatcaaaccgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 100 - DNA, 646 bp, homo sapiens
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctccctccattcactttcggccctgggaccaaagtggatatcaaaccgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 101 - DNA, 42 bp, homo sapiens
tggaccctgtggacacagccacatattactgtgcacacagac

SEQ ID NO: 102 - DNA, 90 bp, homo sapiens
tggaccctgtggacacagccacatatttttgtgcacacaagaaccttcagtattcggaatggttcgacccctggggccagggcaccctgg

SEQ ID NO: 103 - DNA, 51 bp, homo sapiens
acaactggttcgactcctggggccaaggaaccctggtcaccgtctcctcag

(19) United States
(12) Patent Application Publication
(10) Pub. No.: US 2025/0104804 A1
(43) Pub. Date: Mar. 27, 2025

(54) SYSTEMS AND METHODS OF DEVELOPING DISEASE ASSOCIATED ANTIBODY SEQUENCES

(71) Applicant: Absci Corporation, Vancouver, WA (US)

(72) Inventors: Daniele Biasci, Poole, Dorset (GB); Ines De Santiago Domingos De Jesus, London (GB); Berke Cagkan Toptas, Cambridge, MA (US); Goran Rakocevic, Vancouver, WA (US)

(21) Appl. No.: 18/265,738

(22) PCT Filed: Dec. 6, 2021
(86) PCT No.: PCT/US21/62027

§ 371 Date: Nov. 29, 2023

Related U.S. Application Data
(60) Provisional application No. 63/122,406, filed on Dec. 7, 2020.

(51) Int. Cl. G16B 15/30; C12N 15/10; G16B 35/00

(52) U.S. Cl. CPC G16B 15/30; C12N 15/1089; G16B 35/00

(57) ABSTRACT

The disclosure herein relates to in silico methods for reconstructing complete polypeptide and nucleic acid consensus sequences for novel biologically active protein dimers, including but not limited to antibodies that are useful for the treatment and diagnosis of a cancer, autoimmune condition, or infectious disease.

### REFERENCE TO A SEQUENCE LISTING

The present application includes a Sequence Listing which has been submitted electronically in ASCII format and is hereby incorporated by reference in its entirety. Said ASCII copy, created on Dec. 6, 2021, is named 57301_Seglisting.txt and is 67,012 bytes in size.

### BACKGROUND OF THE INVENTION

Within the genome of humans as well as other organisms are multiple loci that produce polypeptides which join together to form dimer structures with biologically active properties. Exemplary protein structures include human immunoglobulins, which comprise a pair of dimer heavy chain and light chain polypeptides, and human T cell receptors, which form a dimer comprising either an alpha and beta chain polypeptide, or a gamma and delta chain polypeptide.

### SUMMARY OF THE INVENTION

This disclosure describes embodiments of systems and methods for producing novel protein compositions which are biologically active and useful for treating patients suffering from a range of disease conditions. In one aspect, the novel protein composition comprises a protein dimer. The protein dimer may be identified by reconstructing polypeptide sequences contained within (e.g.) ribonucleic acid sequencing data isolated from patients having a disease or disorder.

Certain embodiments of the present disclosure recognize and take advantages of two elements: 1) the existence of a small number of cancer, autoimmune, or infectious disease patients with a highly oligoclonal antibody repertoire, and 2) a specialized bioinformatics platform facilitating identification and analysis of such patients. Samples from cancer, autoimmune, or infectious disease patients can be processed according to embodiments of the disclosure to generate RNA sequencing data, and generated sequences from patients can be analyzed to identify treatment candidates.

Another advantage of the present disclosure is that it provides for the generation of fully human antibodies that are candidates for treating various diseases such as cancer. Accordingly, there is no need for the traditional humanization process or laboratory wet steps (e.g., phage display) that are required in classical immunological methods. Instead, the in silico reconstructed consensus sequence is fully human, which can be incorporated directly into a pharmaceutical composition or medicament without the need for further bioengineering.

Another advantage of the present disclosure is the ability to generate sequences for antibodies or antigen binding fragments thereof for treating human diseases or conditions in silico without requiring classical immunological methods. For example, the classical approach is labor-intensive and requires having the purified target antigen to generate antibodies that target the antigen. By contrast, systems and methods of the present disclosure may utilize bioinformatics techniques to reconstruct the sequences of intratumoral antibodies directly from ribonucleic acid sequencing data (e.g., RNA-Seq data).

In one embodiment, a method of inferring protein dimers associated with a disease or disorder from mRNA sequencing data comprises obtaining ribonucleic acid sequence data for a plurality of biological samples obtained from subjects having the disease or disorder. The method further comprises processing the ribonucleic acid sequence data to identify a plurality of mRNA isoforms and inferring at least one protein dimer from the plurality of unique mRNA isoforms. The at least one protein dimer can comprise a first protein isoform and a second protein isoform inferred from the plurality of mRNA isoforms. A consensus sequence may then be reconstructed that codes for the at least one protein dimer based on the plurality of mRNA isoforms.

In some embodiments, the protein dimer at least partially comprises an immunoglobulin variable heavy chain, wherein the variable heavy chain comprises a reconstructed polypeptide consensus sequence. In some embodiments, the reconstructed polypeptide consensus sequence comprises one or more of a variable heavy chain complementarity-determining region CDR-H1, CDR-H2 or CDR-H3.

In some embodiments, the protein dimer at least partially comprises an immunoglobulin variable light chain, wherein the variable light chain comprises a reconstructed polypeptide consensus sequence. In some embodiments, the reconstructed polypeptide consensus sequence comprises one or more of variable light chain complementarity-determining region CDR-L1, CDR-L2 or CDR-L3.

In some embodiments, the protein dimer is a variable heavy chain and variable light chain within an IgG, IgA, or IgM antibody. In some embodiments, the IgG is IgG1, IgG2, IgG3, IgG4, IgGA1, or IgGA2. In some embodiments, the antibody is a chimeric, humanized, or human antibody. In some embodiments, the antibody is a monoclonal antibody. In some embodiments, the antibody is a multispecific antibody. In some embodiments, the antibody is a multivalent antibody. In some embodiments of the aspects disclosed above, the antigen binding fragment is a Fab, Fab′, Fab′-SH, Fv, scFv, F(ab′)2, or a diabody. In some embodiments, the antibody or antigen binding fragment thereof is recombinant. In some embodiments, the antibody or antigen binding fragment thereof further comprises an enzyme, substrate, cofactor, fluorescent marker, chemiluminescent marker, peptide tag, magnetic particle, drug, or toxin. In some embodiments, the antibody or antigen binding fragment thereof is cytolytic to tumor cells.

In some embodiments, the protein dimer is alpha and beta chain or gamma and delta chain of a human T cell receptor. In some embodiments, the T cell receptor is a chimeric antigen receptor.

In some embodiments of the aspects disclosed herein, the protein dimer inhibits tumor growth. In some embodiments, the tumor is selected from the group consisting of brain cancer, renal cancer, ovarian cancer, prostate cancer, colon cancer, lung cancer, squamous cell carcinoma of head and neck, and melanoma.

In some embodiments of the aspects disclosed herein, the protein dimer neutralizes viral infection. In some embodiments, the neutralized virus may be SARS-CoV-2.

In another aspect, provided herein are the systems and methods for generating a polypeptide sequence comprising a fusion protein comprising the protein dimers of the aspects disclosed above.

In one aspect, provided herein are the systems and methods for generating a polypeptide sequence comprising a chimeric antigen receptor of a T cell comprising, (a) an antigen binding fragment of the aspects disclosed above, (b) a transmembrane domain, and (c) an intracellular signaling domain.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary scheme of computational pipeline used for identifying immunoglobulin clonotypes.

FIG. 2A-2J shows alignment visualization of 5 patients and immunoglobulin sequences. Individual reads obtained from RNA-seq are shown for the 5 selected patients. The aligned germline VDJ segments are shown at the bottom of each track. IGV colors paired-end alignments that deviate from expectations (horizontal colored lines) and the mismatched bases are displayed in color (A as green, C as blue, G as yellow and T as red).

FIG. 3 depicts an exemplary schema of VDJ identification pipeline.

FIG. 4 shows a detailed schema of Somatic VDJ sequence identification.

FIG. 5A shows heavy chain and FIG. 5B shows light chain refined alignment for selected patient compared to the initial alignment. Sudden coverage drop can be observed at D segment of heavy chain and the V-J junction of the light chain.

FIG. 6 shows assembly visualization of a heavy D segment.

FIG. 7 shows an IGV plot of heavy chain with a corrected D segment after the alignment stage.

FIG. 8 illustrates a detailed schema of Germline and CDR sequence identification.

FIG. 9 shows an exemplary method of generating a reconstructed consensus sequence in accordance with an embodiment of the disclosure.

FIG. 10 shows an exemplary method of inferring the presence of a protein dimer in accordance with an embodiment of the disclosure.

FIGS. 11A-B depicts one exemplary embodiment of computational and experimental workflows for processing ribonucleic acid sequence data and experimentally validating identified antibodies, respectively.

FIGS. 12A-E are charts depicting various properties of antibodies identified according to embodiments of the disclosure.

FIG. 13 is a chart depicting the design of a synthetic benchmark that may be used to assess the performance of an antibody reconstruction workflow according to an embodiment of the disclosure.

FIGS. 14A-E are charts depicting the distribution of K_{D} values for antibodies derived from intratumoral Ig and commercially available antibodies against the same antigens according to an embodiment of the disclosure.

FIG. 15 is a chart depicting a histogram of the distribution of reads mapped to reconstructed heavy chains for different TCGA samples.

FIGS. 16A-B are charts illustrating an evaluation of reconstruction performance on synthetic data.

FIG. 17 is a graphical representation of epitope mapping results according to an embodiment of the disclosure.

### DETAILED DESCRIPTION OF THE INVENTION

B cells are a central component of the adaptive immune system, playing a diverse set of roles, including antigen recognition and presentation, antibody production and secretion, as well as having regulatory functions. When analysing the immune response to cancer the major scientific focus has, however, been placed on another type of tumor infiltrating lymphocytes (TIL), the T cells, which are often found in high abundance within the tumor microenvironment (TME). Even though a number of studies indicated the presence of B cells as an important prognostic factor, it is with the discovery of Tertiary Lymphoid Structures (TLS) within tumors (Dieu-Nosjean et al. 2008) (Sautes-Fridman et al. 2011) (Dieu-Nosjean et al. 2016), and their direct implication in immunotherapy response and survival (Dieu-Nosjean et al. 2016; Helmink et al. 2020)(Sautes-Fridman et al. 2011; Petitprez et al. 2020)(Cabrita et al. 2020) that the complex nature and organization of immunity in cancer is being illuminated. TLS are lymphoid formations that develop within solid tumors, whose structure and function mirrors that of secondary lymphoid organs (Sautes-Fridman et al. 2019). TLS contain a T cell rich zones and germinal centers (GC) composed of B cells, follicular dendritic cells, and plasma cells. Within the GCs, B cells compete in binding the antigens captured from the surrounding tumour microenvironment, undergo somatic hypermutation, and class switch recombination. It has been suggested that the function and prognostic value of TLS are highly dependent on the presence of GCs(Sautes-Fridman et al. 2019; Silina et al. 2018)(Posch et al. 2018), and consequently on the successful development of B cells within the GC.

Having recognized the importance of B cells in the immune response to cancer, the inventors faced the problem of determining which antigens are recognized by the antibodies produced by these B cells. To study this problem, the inventors developed various embodiments of methods for sequence reconstruction and pairing of, e.g., immunoglobulin chains of clonally expanded B cell populations from bulk RNA sequencing of solid tumor tissues. In one embodiment, antibodies from a selected subset of cancer RNA sequencing samples from The Cancer Genome Atlas (TCGA) are identified and evaluated for therapeutic potential. Many of these antibodies bind known cancer antigens or genes overexpressed in cancer tissue and often show cancer-specific expression patterns, and thus may potentially be used as novel treatments for disease.

### Computationally Reconstructing Antibodies

Provided herein are systems and methods for reconstructing polypeptide and nucleic acid consensus sequences for cancer associated antibodies. The consensus sequences are reconstructed in silico. The term “polypeptide consensus sequence” as used herein refers to an amino acid sequence which comprises the most frequently occurring amino acid residues at each location in all immunoglobulins of any particular subclass or subunit structure. The polypeptide consensus sequence may be based on immunoglobulins of a particular species or of many species. A polypeptide “consensus” sequence, “consensus” structure, or “consensus” antibody is understood to encompass a human polypeptide consensus sequence as described in certain embodiments provided herein, and to refer to an amino acid sequence which comprises the most frequently occurring amino acid residues at each location in all human immunoglobulins of any particular subclass or subunit structure. The embodiments herein provide consensus human structures and consensus structures, which consider other species in addition to human.

The term, “nucleic acid consensus sequence” as used herein refers to a nucleic acid sequence, which comprises the most frequently occurring nucleotide residues at each location in all immunoglobulin nucleic acid sequence of any particular subclass or subunit structure. The nucleic acid consensus sequence may be based on immunoglobulins of a particular species or of many species. A nucleic acid “consensus” sequence, or “consensus” structure, is understood to encompass a human nucleic acid consensus sequence as described in certain embodiments of this invention, and to refer to a nucleic acid sequence which comprises the most frequently occurring nucleotide residues at each location in all human immunoglobulins nucleic acid of any particular subclass or subunit structure.

Provided herein are consensus human structures and consensus structures of other species in addition to human. Methods to computationally reconstruct the consensus sequences from RNA seq data are described in the examples herein. Non limiting examples of computational tools known in the art for reconstructing full-length antibody repertoires including MIGEC (Shugay et al. 2014), PRESTO (Vander Heiden et al. 2014), MiXCR (Bolotin et al. 2015), and IGREPERTOIRECONSTRUCTOR (Safonova et al. 2015). In some embodiments, the TraCeR pipeline by Stubbington and Teichmann is implemented, which uses de novo assembly after a pre-filtering step against a custom database containing in silico combinations for all known human V and J gene segments/alleles in the International Immunogenetics Information System (IMGT) repository. In some embodiments, another pipeline, VDJPuzzle, is implemented which filters in reads by mapping to TCR genes followed by a Trinity-based assembly; whereby the total reads are then mapped back to the assemblies in order to retrieve reads missed in the initial mapping step, followed by another round of assembly with Trinity. An exemplary method for computationally reconstructing consensus sequences can comprise somatic sequence identification, manual IGV investigation and (if necessary) correction of somatic vdj sequence and identification of germline sequence and CDR regions.

In some embodiments, RNA-seq FASTQ files retrieved for patients e.g., a cancer patient are recorded and analysed. Kallisto, BWA, MiXCR or other known tools can be used, in some embodiments, to perform a first alignment of RNA-seq samples to reference V, D and J genes of immunoglobulins in order to identify the repertoire present in the samples. In further embodiments, identical CDR3 sequences are identified and grouped in clonotypes (Bolotin D A et al., Nature Methods, 2015; Bolotin D A et al. Nature biotechnology, 2017). VDJtools are used, in some embodiments, (Shugay M. et al. PLoS computational biology, 2015) to filter out non-functional (non-coding) clonotypes and to compute basic diversity statistics. In further embodiments, non-functional clonotypes are identified as those containing a stop codon or frameshift in their receptor sequence. In some embodiments, the diversity of the Ig repertoire is obtained based on the effective number of species which is calculated as the exponent of the Shannon-Wiener Entropy index (MacArthur RH. Biological reviews. 1965).

In some embodiments, further alignments against the immunoglobulin segments present in the samples are performed for viewing the results to explore the frequency distribution of sequence mismatches along the V, D, J gene segments and, in particular in the CDR3 region length statistics. This alignment step can be useful, for example, for summarizing repertoires, as well as offering a detailed view of rearrangements and region alignments for individual query sequences. Exemplary methodology for alignment and assembly is described in the examples herein.

In some embodiments, the immunoglobulin segments present in the samples are identified using IMGT reference files or equivalent. In some instances, the heavy D segment and light V-J junction sequences can be assembled using an assembler. Non limiting examples of assembler known in the art include Trinity and V'DJer. A FASTA file with corrected heavy D and light V-J junction sequences can be generated for each sample in some embodiments. In addition to the assembled FASTA files, germline FASTA files can be generated, for example, by using IgBLAST v1.9.0 [Ye J, et al Nucleic Acids Research, 2013] and the IMGT database. In further embodiments, the somatic FASTA sequence can be input to IgBLAST to obtain the closest segment ids for the heavy and light chain. The germline FASTA can be generated by merging corresponding segment sequences from the IMGT database. The final assembled FASTA sequences can serve as ‘reference’ sequences for the alignment and visualisation steps.

In further embodiments, using the reference files generated from the assembly step, the FASTQs can be aligned in BowTie2 default mode. Other alignment tools, known in the art, for example STAR or TopHat2 can also be used. The output BAM file can be used for IGV visualization and mutations in the patient can be observed.

In further embodiments, the identification of the CDR3 region and corresponding V, D, and J chains from the final assembled FASTA sequences can be done, for example with IgBLAST. The standardized output using version v.1.9.0 of IgBLAST can be delivered by wrapping IgBLASTn with default parameters in some instances. In other instances, the output from the IgBLAST service can be extracted using a purpose-built parser tool designed to extract the CDR1, CDR2 and CDR3 nucleotide and amino acid sequences.

### Cancer Associated Antibodies or Antigen Binding Fragments Thereof

In another aspect, the present disclosure provides systems and methods for generating cancer associated antibodies comprising a reconstructed consensus sequence. In some embodiments, the antibodies or antigen binding fragment thereof induce lysis of cancer cells. Lysis can be induced by any mechanism, such as by mediating an effector function, such as C1q binding and complement dependent cytotoxicity (CDC); Fc receptor binding; antibody-dependent cell-mediated cytotoxicity (ADCC); phagocytosis, or direct induction of cell apoptosis.

In some embodiments, disclosed herein are systems and methods for generating an antibody or antigen binding fragment thereof, disclosed herein, that is engineered to have at least one increase in effector function as compared to the non-engineered parent antibody or antigen binding fragment thereof. Effector functions are biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis. For example, an antibody or antigen binding fragment thereof, disclosed herein can be glycoengineered to have at least one increase in effector function as compared to the non-glycoengineered parent. Antibody-dependent cellular cytotoxicity (ADCC) is the result of the formation of a complex between the IgG Fab portion of the antibody with the viral protein on the cell surface and binding of the Fc portion to the Fc receptors (FcγRs), on effector cells. The increase in effector function can be increased binding affinity to an Fc receptor, increased ADCC; increased cell mediated immunity; increased binding to cytotoxic CD8 T cells; increased binding to NK cells; increased binding to macrophages; increased binding to polymorphonuclear cells; increased binding to monocytes; increased binding to macrophages; increased binding to large granular lymphocytes; increased binding to granulocytes; direct signaling inducing apoptosis; increased dendritic cell maturation; or increased T cell priming.

### Antibodies

The present disclosure provides systems and methods for generating reconstructed polypeptide consensus sequences for cancer associated antibodies that find use in treating and/or diagnosing cancer. The term “cancer associated antibody” as used herein refers to an antibody specific for a cancer associated antigen. In some embodiments, the cancer associated antibody comprises at least one antigen binding region specific for a cancer associated antigen. Disclosed herein are the complete reconstructed nucleic acid consensus sequences and complete reconstructed polypeptide consensus sequences of the variable heavy chain (VH) and variable light chain (VL) of the antibodies. The nucleic acid and polypeptide sequences of the CDR3 of the VH and the VL are also provided.

An antibody includes monoclonal antibodies, multispecific antibodies (for example, bispecific antibodies and polyreactive antibodies), and antibody fragments. Thus, an antibody includes, but not be limited to, any specific binding member, immunoglobulin class and/or isotype (e.g., IgG1, IgG2, IgG3, IgG4, IgM, IgA, IgD, IgE and IgM); and biologically relevant fragment or specific binding member thereof, including but not limited to Fab, F(ab′)2, Fv, and scFv (single chain or related entity). A monoclonal antibody is obtained from a population of substantially homogeneous antibodies, e.g., the individual antibodies comprising the population are identical except for possible naturally occurring mutations that may be present in minor amounts. A polyclonal antibody is a preparation that includes different antibodies directed against different determinants (epitopes).

It is understood in the art that an antibody is a glycoprotein having at least two heavy (H) chains and two light (L) chains interconnected by disulfide bonds, or an antigen binding portion thereof. A heavy chain is comprised of a heavy chain variable region (VH) and a heavy chain constant region (CH1, CH2 and CH3). A light chain is comprised of a light chain variable region (VL) and a light chain constant region (CL). The variable regions of both the heavy and light chains comprise framework regions (FRs or FWRs) and hypervariable regions (HVRs). The HVRs are the amino acid residues of an antibody that are responsible for antigen binding. The hypervariable region generally comprises amino acid residues from a complementarity determining region (CDR), which have the highest sequence variability and/or involved in antigen recognition. With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See, e.g., Fransson, Front. Biosci. 13:1619-1633 (2008).)

Unless otherwise indicated, HVR residues and other residues in the variable domain (e.g., FR residues) are numbered herein according to Kabat et al., supra. A variable region is a domain of an antibody heavy or light chain that is involved in binding the antibody to antigen. (See, e.g., Kindt et al. Kuby Immunology, 6th ed., W.H. Freeman and Co., p. 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. (See, e.g., Portolano et al., J. Immunol. 150:880-887 (1993); Clarkson et al., Nature 352:624-628 (1991)). The four FWR regions are typically more conserved while CDR regions (CDR1, CDR2 and CDR3) represent hypervariable regions and are arranged from NH2 terminus to the COOH terminus as follows: FWR1, CDR1, FWR2, CDR2, FWR3, CDR3, and FWR4. The variable regions of the heavy and light chains contain a binding domain that interacts with an antigen while, depending of the isotype, the constant region(s) may mediate the binding of the immunoglobulin to host tissues or factors. An antibody also includes chimeric antibodies, humanized antibodies, and recombinant antibodies, human antibodies generated from a transgenic non-human animal, as well as antibodies selected from libraries using enrichment technologies available to the artisan.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Antibody Properties

### Mutation Frequency

The systems and methods of the present disclosure can generate antibodies or antigen binding fragments thereof that comprise a heavy chain sequence with a mutation frequency of at least about 5%, 6%, 7%, 8%, 9%, 10%, 11%, 12%, 13%, 14%, 15%, 16%, 17%, 18%, 19%, or 20%, or higher from a germline sequence. In some embodiments, the reconstructed germline polypeptide sequences of the antibodies or antigen binding fragment thereof of the disclosure can be selected from Table 5.

The antibodies of the present disclosure can comprise a CDR3 region that is a light chain sequence with a mutation frequency of at least about 5%, 6%, 7%, 8%, 9%, 10%, 11%, 12%, 13%, 14%, 15%, 16%, 17%, 18%, 19%, or 20%, or higher from a germline sequence. The antibodies of the present disclosure can comprise a CDR1 region that is a light chain sequence with a mutation frequency of at least about 5%, 6%, 7%, 8%, 9%, 10%, 11%, 12%, 13%, 14%, 15%, 16%, 17%, 18%, 19%, or 20%, or higher from a germline sequence. The antibodies of the present disclosure can comprise a CDR2 region that is a light chain sequence with a mutation frequency of at least about 5%, 6%, 7%, 8%, 9%, 10%, 11%, 12%, 13%, 14%, 15%, 16%, 17%, 18%, 19%, or 20%, or higher from a germline sequence.

The antibodies of the present disclosure can comprise a CDR3 region that is a heavy chain sequence with a mutation frequency of at least about 5%, 6%, 7%, 8%, 9%, 10%, 11%, 12%, 13%, 14%, 15%, 16%, 17%, 18%, 19%, or 20%, or higher from a germline sequence. The antibodies of the present disclosure can comprise a CDR1 region that is a heavy chain sequence with a mutation frequency of at least about 5%, 6%, 7%, 8%, 9%, 10%, 11%, 12%, 13%, 14%, 15%, 16%, 17%, 18%, 19%, or 20%, or higher from a germline sequence. The antibodies of the present disclosure can comprise a CDR2 region that is a heavy chain sequence with a mutation frequency of at least about 5%, 6%, 7%, 8%, 9%, 10%, 11%, 12%, 13%, 14%, 15%, 16%, 17%, 18%, 19%, or 20%, or higher from a germline sequence.

The antibodies or antigen binding fragment thereof of the invention can comprise a heavy chain and a light chain sequence with a mutation frequency of at least about 5%, 6%, 7%, 8%, 9%, 10%, 11%, 12%, 13%, 14%, 15%, 16%, 17%, 18%, 19%, or 20%, or higher from a germline sequence. The antibodies or antigen binding fragment thereof of the invention can comprise a VH region from a VH family selected from the group consisting of any one of VH family 4-59.

### Heavy and Light Chain Lengths

The systems and methods of the present disclosure can generate antibodies or antigen binding fragments thereof that comprise a CDR3 region that is a length of at least about 10, 11, 12, 13, 14, 15, 16, 17, 18, 19, 20, 21, 22, 23, 24, 25, 26, 27, 28, 29, or 30 amino acids in length. The antibodies or antigen binding fragment thereof of the present disclosure can comprise a CDR3 region that is at least about 18 amino acids in length.

The systems and methods of the present disclosure can generate antibodies or antigen binding fragments thereof that comprise a deletion at an end of a light chain. The antibodies or antigen binding fragment thereof of the invention can comprise a deletion of 3 or more amino acids at an end of the light chain. The antibodies or antigen binding fragment thereof of the invention can comprise a deletion of 7 or less amino acids at an end of the light chain. The antibodies or antigen binding fragment thereof of the invention can comprise a deletion of 3, 4, 5, 6, or 7 amino acids at an end of the light chain.

The systems and methods of the present disclosure can generate antibodies or antigen binding fragments thereof that comprise an insertion in a light chain. The antibodies or antigen binding fragment thereof of the invention can comprise an insertion of 1, 2, 3, 4, 5, 6, 7, 8, 9, or 10, or more amino acids in the light chain. The antibodies or antigen binding fragment thereof of the invention can comprise an insertion of 3 amino acids in the light chain.

### Affinity

Affinity is the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (k_{d}). Affinity can be measured by common methods known in the art, including those described herein. Specific illustrative and exemplary embodiments for measuring binding affinity are described in the following.

In some embodiments, systems and methods of the present disclosure can generate a reconstructed consensus sequence corresponding to at least a portion of an antibody that has a dissociation constant (K_{D}) of about 1 μM, 100 nM, 10 nM, 5 nM, 2 nM, 1 nM, 0.5 nM, 0.1 nM, 0.05 nM, 0.01 nM, or 0.001 nM or less (e.g., 10⁻⁸M or less, e.g., from 10⁻⁸M to 10⁻³M, e.g., from 10⁻⁹M to 10⁻¹³ M). Another aspect of the invention provides for an antibody or antigen binding fragment thereof with an increased affinity for its target, for example, an affinity matured antibody. An affinity matured antibody is an antibody with one or more alterations in one or more hypervariable regions (HVRs), compared to a parent antibody which does not possess such alterations, such alterations resulting in an improvement in the affinity of the antibody for antigen. These antibodies can bind to antigen with a K_{D} of about 5×10⁻⁹M, 2×10⁻⁹M, 1×10⁻⁹M, 5×10⁻¹⁰ M, 2×10⁻⁹M, 1×10⁻¹⁰M, 5×10⁻¹¹ M, 1×10⁻¹² M, 5×10⁻¹² M, 1×10⁻¹² M, or less. In some embodiments, the present disclosure provides an antibody or antigen binding fragment thereof which has an increased affinity of at least 1.5 fold, 2 fold, 2.5 fold, 3 fold, 4 fold, 5 fold, 10 fold, 20 fold or greater as compared to a germline antibody containing the heavy chain sequence and light chain sequence, or both. In other embodiments, an antibody is provided that competes for binding to the same epitope as an antibody as described herein. In some embodiments, the antibody or antigen binding fragment thereof that binds to the same epitope, and/or competes for binding to the same epitope as an antibody exhibits effector function activities, such as, for example, Fc-mediated cellular cytotoxicity, including ADCC activity.

K_{D} can be measured by any suitable assay. For example, K_{D} can be measured by a radiolabeled antigen binding assay (RIA) (See, e.g., Chen et al., J. Mol. Biol. 293:865-881 (1999); Presta et al., Cancer Res. 57:4593-4599 (1997)). For example, K_{D} can be measured using surface plasmon resonance assays (e.g., using a BIACORE®-2000 or a BIACORE®-3000).

### Antibody Fragments

An antibody fragment or “antigen binding fragment” comprises a portion of an intact antibody, such as the antigen binding or variable region of the intact antibody. In a further aspect of the invention, an antibody according to any of the above embodiments is a monoclonal antibody, including a chimeric, humanized or human antibody. Antibody fragments include, but are not limited to, Fab, Fab′, Fab′-SH, F(ab′)₂, Fv, diabody, linear antibodies, multispecific formed from antibody fragments antibodies and scFv fragments, and other fragments described below. In another embodiment, the antibody is a full length antibody, e.g., an intact IgG1 antibody or other antibody class or isotype as described herein. (See, e.g., Hudson et al. Nat. Med. 9:129-134 (2003); Pluckthiin, The Pharmacology of Monoclonal Antibodies, vol. 113, pp. 269-315 (1994); Hollinger et al., Proc. Natl. Acad. Sci. USA 90: 6444-6448 (1993)). A full length antibody, intact antibody, or whole antibody is an antibody having a structure substantially similar to a native antibody structure or having heavy chains that contain an Fc region as defined herein. Antibody fragments can be made by various techniques, including but not limited to proteolytic digestion of an intact antibody as well as production by recombinant host cells (e.g., E. coli or phage), as described herein.

An Fv is the minimum antibody fragment that contains a complete antigen-recognition and antigen-binding site. This fragment contains a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable region (or half of an Fv comprising only three CDRs specific for an antigen) has the ability to recognize and bind antigen, although at a lower affinity than the entire binding site.

A single-chain Fv (sFv or scFv) is an antibody fragment that comprises the V_{H} and V_{L} antibody domains connected into a single polypeptide chain. The sFv polypeptide can further comprise a polypeptide linker between the V_{H} and V_{L} domains that enables the sFv to form the desired structure for antigen binding. (See, e.g., Pluckthun in The Pharmacology of Monoclonal Antibodies, vol. 113, Rosenburg and Moore eds., Springer-Verlag, New York, pp. 269-315 (1994); Borrebaeck 1995, infra.

A diabody is a small antibody fragment prepared by constructing an sFv fragment with a short linker (about 5-10 residues) between the V_{H} and V_{L} domains such that inter-chain but not intra-chain pairing of the V domains is achieved, resulting in a bivalent fragment. Bispecific diabodies are heterodimers of two crossover sFv fragments in which the V_{H} and V_{L} domains of the two antibodies are present on different polypeptide chains. (See, e.g., Hollinger et al, Proc. Natl. Acad. Sci. USA, 90:6444-6448 (1993)).

Domain antibodies (dAbs), which can be produced in fully human form, are the smallest known antigen-binding fragments of antibodies, ranging from about 11 kDa to about 15 kDa. DAbs are the robust variable regions of the heavy and light chains of immunoglobulins (V_{H} and V_{L}, respectively). They are highly expressed in microbial cell culture, show favorable biophysical properties including, for example, but not limited to, solubility and temperature stability, and are well suited to selection and affinity maturation by in vitro selection systems such as, for example, phage display. DAbs are bioactive as monomers and, owing to their small size and inherent stability can be formatted into larger molecules to create drugs with prolonged serum half-lives or other pharmacological activities.

Fv and sFv are the only species with intact combining sites that are devoid of constant regions. Thus, they are suitable for reduced nonspecific binding during in vivo use. sFv fusion proteins can be constructed to yield fusion of an effector protein at either the amino or the carboxy terminus of an sFv. The antibody fragment also can be a “linear antibody. Such linear antibody fragments can be monospecific or bispecific.

### Human Antibodies

In some embodiments, the systems and methods disclosed herein provide for the generation of reconstructed consensus sequences coding for an antibody provided herein that is a human antibody. Human antibodies can be produced using various techniques known in the art (See, e.g., van Dijk and van de Winkel, Curr. Opin. Pharmacol. 5: 368-74 (2001); and Lonberg, Curr. Opin. Immunol. 20:450-459 (2008)). A human antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies may be prepared by administering an immunogen (e.g., a cancer cell antigen) to a transgenic animal that has been modified to produce intact human antibodies or intact antibodies with human variable regions in response to antigenic challenge. (See, e.g., Lonberg, Nat. Biotech. 23:1117-1125 (2005)). Human variable regions from intact antibodies generated by such animals may be further modified, e.g., by combining with a different human constant region.

Human antibodies can also be made by hybridoma-based methods. For example, human antibodies can be produced from human myeloma and mouse-human heteromyeloma cell lines, using human B-cell hybridoma technology, and other methods (See, e.g., Kozbor J. Immunol., 133: 3001 (1984); Brodeur et al., Monoclonal Antibody Production Techniques and Applications, pp. 51-63 (1987); Boerner et al., J. Immunol., 147: 86 (1991); Li et al., Proc. Natl. Acad., 103:3557-3562 (2006); Ni, Xiandai Mianyixue, 26(4):265-268 (2006); Vollmers and Brandlein, Histology and Histopathology, 20(3):927-937 (2005); and Vollmers and Brandlein, Methods and Findings in Experimental and Clinical Pharmacology, 27(3):185-91 (2005)). Human antibodies may also be generated by isolating Fv clone variable domain sequences selected from human-derived phage display libraries. Such variable domain sequences may then be combined with a desired human constant domain.

The systems and methods of the present disclosure enable in silico generation of human antibody sequences (e.g., polypeptide sequences) without requiring wet laboratory steps.

### Library-Derivation

Antibodies or antigen binding fragment thereof of the present disclosure may be isolated by screening combinatorial libraries for antibodies with the desired activity or activities. (See, e.g., in Hoogenboom et al., Methods in Molecular Biology 178:1-37 (2001); McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004)). Repertoires of V_{H} and V_{L} genes can be cloned separately (e.g., by PCR) and recombined randomly in libraries (e.g., phage libraries), and screened (See, e.g., Winter et al., Ann. Rev. Immunol., 12: 433-455 (1994)). Alternatively, the naive repertoire can be cloned (e.g., from human) to provide a single source of antibodies to a wide range of non-self and also self-antigens without any immunization (See, e.g., Griffiths et al., EMBO J, 12: 725-734 (1993). Alternatively, naive libraries can be synthetically made by cloning unrearranged V-gene segments from stem cells, and encoding the CDR3 regions using random primers or to rearrange the V-gene segments in vitro (See, e.g., Hoogenboom and Winter, J. Mol. Biol., 227: 381-388 (1992)). Antibodies or antibody fragments isolated from human antibody libraries are considered human antibodies or human antibody fragments herein.

### Multispecificity

In some embodiments, an antibody provided herein is a multispecific antibody, e.g., a bispecific antibody. Multispecific antibodies are monoclonal antibodies that have binding specificities for at least two different sites. In some embodiments, one of the binding specificities is for cancer associated antigen and the other is for any other antigen. In some embodiments, bispecific antibodies may bind to two different epitopes of antigen. Bispecific antibodies may also be used to localize cytotoxic agents to cancer cells. Bispecific antibodies can be prepared as full length antibodies or antibody fragments.

Exemplary techniques for making multispecific antibodies include recombinant co-expression of two immunoglobulin heavy chain-light chain pairs having different specificities, engineering electrostatic steering effects for making antibody Fc-heterodimeric molecules, cross-linking two or more antibodies or fragments, using leucine zippers to produce bi-specific antibodies, using “diabody” technology for making bispecific antibody fragments, using single-chain Fv (sFv) dimers, preparing trispecific antibodies, and “knob-in-hole” engineering (See, e.g., Milstein and Cuello, Nature 305: 537 (1983); Traunecker et al., EMBO J. 10: 3655 (1991); U.S. Pat. Nos. 4,676,980 and 5,731,168; Brennan et al., Science, 229: 81 (1985); Kostelny et al., J. Immunol., 148(5):1547-1553 (1992); Hollinger et al., Proc. Natl. Acad. Sci. USA, 90:6444-6448 (1993); Gruber et al., J. Immunol., 152:5368 (1994)); and Tutt et al. J. Immunol. 147: 60 (1991)). Engineered antibodies with three or more functional antigen binding sites are also contemplated.

### Variants

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of techniques well known in the art. For example, it may be desirable to improve the binding affinity and/or other biological properties of the antibody amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In some embodiments, the systems and methods of the present disclosure generate antibody variants or antigen binding fragments thereof having one or more amino acid substitutions are provided. Sites of interest for mutagenesis by substitution include the CDRs and FRs. Amino acid substitutions may be introduced into an antibody of interest and the products screened for a desired activity, e.g., retained/improved antigen binding, decreased immunogenicity, or improved ADCC or CDC function.

| Original Residue | Exemplary Conserved Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Hydrophobic amino acids include: Norieucine, Met, Ala, Val, Leu, and Ile. Neutral hydrophilic amino acids include: Cys, Ser, Thr, Asn, and Gin. Acidic amino acids include: Asp and Glu. Basic amino acids include: His, Lys, and Arg. Amino acids with residues that influence chain orientation include: Gly and Pro. Aromatic amino acids include: Trp, Tyr, and Phe.

In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots” or SDRs. In some embodiments of the variant V_{H} and V_{L} sequences, each CDR either is unaltered, or contains no more than one, two or three amino acid substitutions.

Alterations (e.g., substitutions) may be made in CDRs, e.g., to improve antibody affinity. Such alterations may be made in CDR encoding codons with a high mutation rate during somatic maturation (See, e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See, e.g., Hoogenboom et al. in Methods in Molecular Biology 178:1-37 (2001)). CDR residues involved in antigen binding may be specifically identified, e.g., using alanine scanning mutagenesis or modeling (See, e.g., Cunningham and Wells Science, 244:1081-1085 (1989)). CDR-H3 and CDR-L3 in particular are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to a polypeptide which increases serum half life of the antibody, for example, at the N-terminus or C-terminus. The term “epitope tagged” refers to the antibody fused to an epitope tag. The epitope tag polypeptide has enough residues to provide an epitope against which an antibody there against can be made, yet is short enough such that it does not interfere with activity of the antibody. The epitope tag preferably is sufficiently unique so that the antibody there against does not substantially cross-react with other epitopes. Suitable tag polypeptides generally have at least 6 amino acid residues and usually between about 8-50 amino acid residues (preferably between about 9-30 residues). Examples include the flu HA tag polypeptide and its antibody 12CA5 [Field et al., Mal. Cell. Biol. 8: 2159-2165 (1988)]; the c-myc tag and the 8F9, 3C7, 6E10, G4, B7 and 9E10 antibodies thereto [Evan et al., Mal. Cell. Biol. 5(12): 3610-3616 (1985)]; and the Herpes Simplex virus glycoprotein D (gD) tag and its antibody [Paborsky et al., Protein Engineering 3(6): 547-553 (1990)]. Other exemplary tags are a poly-histidine sequence, generally around six histidine residues, that permits isolation of a compound so labeled using nickel chelation. Other labels and tags, such as the FLAG® tag (Eastman Kodak, Rochester, N.Y.), well known and routinely used in the art, are embraced by the invention.

Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

### Fc Region Variants

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model (See, e.g., Clynes et al. Proc. Nat'l Acad. Sci. USA 95:652-656 (1998). C1q binding assays may also be carried out to confirm that the antibody is able or unable bind C1q and hence contains or lacks CDC activity (Idusogie et al. J. Immunol. 164: 4178-4184 (2000)). To assess complement activation, a CDC assay may be performed (See, e.g., Gazzano-Santoro et al., J. Immunol. Methods 202:163 (1996); Cragg, M. S. et al., Blood 101:1045-1052 (2003); and Cragg et al., Blood 103:2738-2743 (2004)). FcRn binding and in vivo clearance/half-life determinations can also be performed using methods known in the art (See, e.g., Petkova, S. B. et al., Int'l. Immunol. 18(12):1759-1769 (2006)). Antibodies with reduced effector function include those with substitution of one or more of Fc region residues 238, 265, 269, 270, 297, 327 and 329; or two or more of amino acid positions 265, 269, 270, 297 and 327, such as an Fc mutant with substitution of residues 265 and 297 to alanine (See, e.g., U.S. Pat. Nos. 6,737,056 and 7,332,581). Antibody variants with improved or diminished binding to FcRs are also included (See, e.g., Shields et al., J. Biol. Chem. 9(2): 6591-6604 (2001)). In some embodiments, an antibody variant comprises an Fc region with one or more amino acid substitutions which improve ADCC, e.g., substitutions at positions 298, 333, and/or 334 of the Fc region.

Antibodies can have increased half-lives and improved binding to the neonatal Fc receptor (FcRn). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434. Other examples of Fc region variants are also contemplated (See, e.g., Duncan & Winter, Nature 322:738-40 (1988)).

### Cysteine Engineered Antibody Variants

In some embodiments, it may be desirable to create cysteine engineered antibodies or antigen binding fragment thereof, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker-drug moieties, to create an immunoconjugate. In some embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described.

### Bispecific Antibodies

In some embodiments, it may be desirable to generate multispecific (e.g. bispecific) monoclonal antibody including monoclonal, human, humanized, or variant antibodies having binding specificities for at least two different epitopes. In some embodiments, the antibodies disclosed herein are multispecific. Exemplary bispecific antibodies may bind to two different epitopes of an antigen (e.g., cancer associated antigen). Alternatively, an antigen binding region may be combined with a region which binds to a triggering molecule on a leukocyte such as a T-cell receptor molecule (e.g., CD2 or CD3), or Fe receptors for IgG (FcγR), such as FcγRI (CD64), FcγRII (CD32) and FcγRIII (CD16) so as to focus cellular defense mechanisms to the antigen-expressing cell. Bispecific antibodies may also be used to localize cytotoxic agents to cells which express desired antigen. These antibodies possess an antigen-binding arm and an arm which binds the cytotoxic agent (e.g., saporin, anti-interferon-60, vinca alkaloid, ricin A chain, methotrexate or radioactive isotope hapten). Bispecific antibodies can be prepared as full length antibodies or antibody fragments (e.g., F(ab′)₂ bispecific antibodies).

According to another approach for making bispecific antibodies, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are contemplated, along with a number of cross-linking techniques.

### Monoclonal Antibodies

In some embodiments, the antibodies of the present disclosure are monoclonal. Monoclonal antibodies may be made using the hybridoma method first described by Kohler et al., Nature, 256:495 (1975), or may be made by recombinant DNA methods.

### Engineered and Modified Antibodies

An antibody according to at least some embodiments of the invention further can be prepared using an antibody having one or more of the VH and/or VL sequences derived from an antibody or antigen binding fragment thereof, disclosed herein, starting material to engineer a modified antibody, which modified antibody may have altered properties from the starting antibody. Provided herein are complete reconstructed amino acid and nucleic acid consensus sequences of VH and VL chain regions of antibodies disclosed herein. Also provided herein, are the amino acid and nucleic acid sequences of the CDR3 regions of the VH and VL of the antibodies, described herein. An antibody can be engineered by modifying one or more residues within one or both variable regions (e.g., VH and/or VL), for example within one or more CDR regions and/or within one or more framework regions. Additionally or alternatively, an antibody can be engineered by modifying residues within the constant regions, for example, to alter the effector functions of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific antibodies by constructing expression vectors that include CDR sequences from the specific antibody (e.g. antibodies disclosed herein) grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Suitable framework sequences can be obtained from public DNA databases or published references that include germline antibody gene sequences. For example, germline DNA sequences for human heavy and light chain variable region genes can be found in the “VBase” human germline sequence database (available on the Internet), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline VH Sequences Reveals about Fifty Groups of VH Segments with Different Hypervariable Loops” J. Mol. Biol. 227:776-798; and Cox, J. P. L. et al. (1994) “A Directory of Human Germ-line VH Segments Reveals a Strong Bias in their Usage” Eur. J. Immunol. 24:827-836; the contents of each of which are expressly incorporated herein by reference.

Another type of variable region modification is to mutate amino acid residues within the VH and/or VL CDR 1, CDR2 and/or CDR3 regions to thereby improve one or more binding properties (e.g., affinity) of the antibody of interest. Site-directed mutagenesis or PCR-mediated mutagenesis can be performed to introduce the mutations and the effect on antibody binding, or other functional property of interest, can be evaluated in appropriate in vitro or in vivo assays. Preferably conservative modifications (as discussed above) are introduced. The mutations may be amino acid substitutions, additions or deletions, but are preferably substitutions. Moreover, typically no more than one, two, three, four or five residues within a CDR region are altered.

Engineered antibodies according to at least some embodiments of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived.

In addition or alternative to modifications made within the framework or CDR regions, antibodies according to at least some embodiments of the disclosure may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described above. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody. In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. In another example, one or more amino acids can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al. In another example, one or more amino acid residues are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fey receptor by modifying one or more amino acids. This approach is described further in PCT Publication WO 00/42072 by Presta. Moreover, the binding sites on human IgG1 for Fc gamma RI, Fc gamma RII, Fc gammaRIII and FcRn have been mapped and variants with improved binding have been described (see Shields, R. L. et al. (2001) J. Biol. Chem. 276:6591-6604). Specific mutations at positions are shown to improve binding to FcγRIII. Furthermore, specific mutations such as may improve binding to FcRn and increase antibody circulation half-life (see Chan C A and Carter P J (2010) Nature Rev Immunol 10:301-316). In some embodiments, the constant region of the antibodies disclosed herein are replaced with IGHG1.

In still another embodiment, the glycosylation of an antibody is modified. For example, an aglycoslated antibody can be made (e.g., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Conservative substitutions involve replacing an amino acid with another member of its class. Non-conservative substitutions involve replacing a member of one of these classes with a member of another class.

Any cysteine residue not involved in maintaining the proper conformation of the monoclonal, human, humanized, or variant antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

Other modifications of the antibody are contemplated. For example, it may be desirable to modify the antibody of the invention with respect to effector function, so as to enhance the effectiveness of the antibody in treating cancer, for example. For example cysteine residue(s) may be introduced in the Fe region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176: 1191-1195 (1992) and Shapes, B. J. Immunol. 148: 2918-2922 (1992). Homodimeric antibodies with enhanced anti-tumor activity may also be prepared using heterobifunctional cross-linkers as described in Wolff et al., Cancer Research 53: 2560-2565 (1993). Alternatively, an antibody can be engineered which has dual Fe regions and may thereby have enhanced complement lysis and ADCC capabilities. See Stevenson et al., Anti-Cancer Drug Design 3: 219-230 (1989). In addition, it has been shown that sequences within the CDR can cause an antibody to bind to MHC Class II and trigger an unwanted helper T-cell response. A conservative substitution can allow the antibody to retain binding activity yet lose its ability to trigger an unwanted T-cell response. Also see Steplewski et al., Proc Natl Acad Sci USA. 1988; 85(13):4852-6, incorporated herein by reference in its entirety, which described chimeric antibodies wherein a murine variable region was joined with human gamma 1, gamma 2, gamma 3, and gamma 4 constant regions.

In certain embodiments of the invention, it may be desirable to use an antibody fragment, rather than an intact antibody, to increase tumor penetration, for example. In this case, it may be desirable to modify the antibody fragment in order to increase its serum half-life, for example, adding molecules such as PEG or other water soluble polymers, including polysaccharide polymers, to antibody fragments to increase the half-life. This may also be achieved, for example, by incorporation of a salvage receptor binding epitope into the antibody fragment (e.g., by mutation of the appropriate region in the antibody fragment or by incorporating the epitope into a peptide tag that is then fused to the antibody fragment at either end or in the middle, e.g., by DNA or peptide synthesis) (see, e.g., WO96/32478).

The salvage receptor binding epitope preferably constitutes a region wherein any one or more amino acid residues from one or two loops of a Fe domain are transferred to an analogous position of the antibody fragment. Even more preferably, three or more residues from one or two loops of the Fe domain are transferred. Still more preferred, the epitope is taken from the CH2 domain of the Fe region (e.g., of an igG) and transferred to the CH1, CH3, or V_{H} region, or more than one such region, of the antibody. Alternatively, the epitope is taken from the CH2 domain of the Fe region and transferred to the CL region or VL region, or both, of the antibody fragment. See also International applications WO 97/34631 and WO 96/32478 which describe Fe variants and their interaction with the salvage receptor.

Thus, antibodies of the invention may comprise a human Fe portion, a human consensus Fe portion, or a variant thereof that retains the ability to interact with the Fe salvage receptor, including variants in which cysteines involved in disulfide bonding are modified or removed, and/or in which the a met is added at the N-terminus and/or one or more of the N-terminal 20 amino acids are removed, and/or regions that interact with complement, such as the C1q binding site, are removed, and/or the ADCC site is removed [see, e.g., Malec. Immunol. 29 (5): 633-9 (1992)].

Previous studies mapped the binding site on human and murine IgG for FcR primarily to the lower hinge region composed of lgG residues 233-239. Other studies proposed additional broad segments, e.g. Gly316-Lys338 for human Fe receptor I, Lys274-Arg301 and Tyr407-Arg416 for human Fe receptor III, or found a few specific residues outside the lower hinge, e.g. Asn297 and Glu318 for murine IgG2b interacting with murine Fe receptor II. The report of the 3.2-A crystal structure of the human IgG Fe fragment with human Fe receptor IIIA delineated IgG1 residues Leu234-Ser239, Asp265-Glu269, Asn297-Thr299, and Ala327-Ile332 as involved in binding to Fee receptor IIIA. It has been suggested based on crystal structure that in addition to the lower hinge (Leu234-Gly237), residues in IgG CH2 domain loops FG (residues 326-330) and BC (residues 265-271) might play a role in binding to Fe receptor IIA. See Shields et al., J. Biol. Chem., 276(9):6591-6604 (2001), incorporated by reference herein in its entirety. Mutation of residues within Fe receptor binding sites can result in altered effector function, such as altered ADCC or CDC activity, or altered half-life. As described above, potential mutations include insertion, deletion or substitution of one or more residues, including substitution with alanine, a conservative substitution, a non-conservative substitution, or replacement with a corresponding amino acid residue at the same position from a different IgG subclass (e.g. replacing an IgG1 residue with a corresponding IgG2 residue at that position).

Shields et al. reported that IgG1 residues involved in binding to all human Fe receptors are located in the CH2 domain proximal to the hinge and fall into two categories as follows: 1) positions that may interact directly with all FcR include Leu234-Pro238, Ala327, and Pro329 (and possibly Asp265); 2) positions that influence carbohydrate nature or position include Asp265 and Asn297. The additional IgG 1 residues that affected binding to Fe receptor II are as follows: (largest effect) Arg255, Thr256, Glu258, Ser267, Asp270, Glu272, Asp280, Arg292, Ser298, and (less effect) His268, Asn276, His285, Asn286, Lys290, Gln295, Arg301, Thr307, Leu309, Asn315, Lys322, Lys326, Pro331, Ser337, Ala339, Ala378, and Lys414. A327Q, A327S, P329A, D265A and D270A reduced binding. In addition to the residues identified above for all FcR, additional IgG 1 residues that reduced binding to Fe receptor IIIA by 40% or more are as follows: Ser239, Ser267 (Gly only), His268, Glu293, Gln295, Tyr296, Arg301, Val303, Lys338, and Asp376. Variants that improved binding to FcRIIIA include T256A, K290A, S298A, E333A, K334A, and A339T.

Lys414 showed a 40% reduction in binding for FcRIIA and FcRIIB, Arg416 a 30% reduction for FcRIIA and FcRIIIA, Gln419 a 30% reduction to FcRIIA and a 40% reduction to FcRIIB, and Lys360 a 23% improvement to FcRIIIA. See also Presta et al., Biochem. Soc. Trans. (2001) 30, 487-490.

For example, U.S. Pat. No. 6,194,551, incorporated herein by reference in its entirety, describes variants with altered effector function containing mutations in the human IgG Fe region, at amino acid position 329, 331 or 322 (using Kabat numbering), some of which display reduced C1q binding or CDC activity. As another example, U.S. Pat. No. 6,737,056, incorporated herein by reference in its entirety, describes variants with altered effector or Fe-gamma-receptor binding containing mutations in the human IgG Fe region, at amino acid position 238, 239, 248, 249, 252, 254, 255, 256, 258, 265, 267, 268, 269, 270, 272, 276, 278, 280, 283, 285, 286, 289, 290, 292, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439 (using Kabat numbering), some of which display receptor binding profiles associated with reduced ADCC or CDC activity. Of these, a mutation at amino acid position 238, 265, 269, 270, 327 or 329 are stated to reduce binding to FcRI, a mutation at amino acid position 238, 265, 269, 270, 292, 294, 295, 298, 303, 324, 327, 329, 333, 335, 338, 373, 376, 414, 416, 419, 435, 438 or 439 are stated to reduce binding to FcRII, and a mutation at amino acid position 238, 239, 248, 249, 252, 254, 265, 268, 269, 270, 272, 278, 289, 293, 294, 295, 296, 301, 303, 322, 327, 329, 338, 340, 373, 376, 382, 388, 389, 416, 434, 435 or 437 is stated to reduce binding to FcRIII.

U.S. Pat. No. 5,624,821, incorporated by reference herein in its entirety, reports that C1q binding activity of an murine antibody can be altered by mutating amino acid residue 318, 320 or 322 of the heavy chain and that replacing residue 297 (Asn) results in removal of lytic activity.

United States Application Publication No. 20040132101, incorporated by reference herein in its entirety, describes variants with mutations at amino acid positions 240, 24 245, 247, 262, 263, 266, 299, 313, 325, 328, or 332 (using Kabat numbering) or positions 234, 235, 239, 240, 241, 243, 244, 245, 247, 262, 263, 264, 265, 266, 267, 269, 296, 297, 298, 299, 313, 325, 327, 328, 329, 330, or 332 (using Kabat numbering), of which mutations at positions 234, 235, 239, 240, 241, 243, 244, 245, 247, 262, 263, 264, 265, 266, 267, 269, 296, 297, 298, 299, 313, 325, 327, 328, 329, 330, or 332 may reduce ADCC activity or reduce binding to an Fe gamma receptor.

Chappel et al., Proc Natl Acad Sci USA. 1991; 88(20):9036-40, incorporated herein by reference in its entirety, report that cytophilic activity of IgG 1 is an intrinsic property of its heavy chain CH2 domain. Single point mutations at any of amino acid residues 234-237 of IgG1 significantly lowered or abolished its activity. Substitution of all of lgG1 residues 234-237 (LLGG) into IgG2 and IgG4 were required to restore full binding activity. An IgG2 antibody containing the entire ELLGGP sequence (residues 233-238) was observed to be more active than wild-type IgG1.

Isaacs et al., J Immunol. 1998; 161(8):3862-9, incorporated herein by reference in its entirety, report that mutations within a motif critical for Fe gammaR binding (glutamate 233 to praline, leucine/phenylalanine 234 to valine, and leucine 235 to alanine) completely prevented depletion of target cells. The mutation glutamate 318 to alanine eliminated effector function of mouse IgG2b and also reduced the potency of human IgG4.

Armour et al., Mol Immunol. 2003; 40(9):585-93, incorporated by reference herein in its entirety, identified IgG 1 variants which react with the activating receptor, FcgammaRIIa, at least 10-fold less efficiently than wildtype IgG1 but whose binding to the inhibitory receptor, FcgammaRIIb, is only four-fold reduced. Mutations were made in the region of amino acids 233-236 and/or at amino acid positions 327, 330 and 331. See also WO 99/58572, incorporated by reference herein in its entirety. Xu et al., J Biol Chem. 1994; 269(5):3469-74, incorporated by reference herein in its entirety, report that mutating IgG1 Pro331 to Ser markedly decreased C1q binding and virtually eliminated lytic activity. In contrast, the substitution of Pro for Ser331 in IgG4 bestowed partial lytic activity (40%) to the IgG4 Pro331 variant.

Schuurman et al., Mol Immunol. 2001; 38(1):1-8, incorporated by reference herein in its entirety, report that mutating one of the hinge cysteines involved in the inter-heavy chain bond formation, Cys226, to serine resulted in a more stable inter-heavy chain linkage. Mutating the IgG4 hinge sequence Cys-Pro-Ser-Cys to the IgG1 hinge sequence Cys-Pro-Pro-Cys also markedly stabilizes the covalent interaction between the heavy chains. Angal et al., Mol Immunol. 1993; 30(1):105-8, incorporated by reference herein in its entirety, report that mutating the serine at amino acid position 241 in IgG4 to praline (found at that position in IgG1 and IgG2) led to the production of a homogeneous antibody, as well as extending serum half-life and improving tissue distribution compared to the original chimeric IgG4.

Affinity maturation involves preparing and screening antibody variants that have substitutions within the CDRs of a parent antibody and selecting variants that have improved biological properties such as binding affinity relative to the parent antibody. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity).

Alanine scanning mutagenesis can be performed to identify hypervariable region residues that contribute significantly to antigen binding. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

### Methods of Engineering Antibodies

As discussed above, antibodies having VH and VL sequences disclosed herein can be used to create new antibodies, respectively, by modifying the VH and/or VL sequences, or the constant regions attached thereto. Thus, in another aspect according to at least some embodiments of the present disclosure, the structural features of an antibody disclosed herein according to at least some embodiments of the disclosure, are used to create structurally related antibodies that retain at least one functional property of the parent antibodies according to at least some embodiments of the disclosure herein, such as binding to human cancer cell antigen, respectively. For example, one or more CDR regions of one antibody disclosed herein or mutations thereof, can be combined recombinantly with known framework regions and/or other CDRs to create additional, recombinantly-engineered, antibodies according to at least some embodiments of the disclosure, as discussed above. Other types of modifications include those described in the previous section. The starting material for the engineering method is one or more of the VH and/or VL sequences provided herein, or one or more CDR regions thereof, or one or more of the CDR3 region sequences provided herein. To create the engineered antibody, it is not necessary to actually prepare (e.g., express as a protein) an antibody having one or more of the VH and/or VL sequences provided herein, or one or more CDR regions thereof. Rather, the information contained in the sequences is used as the starting material to create a “second generation” sequences derived from the original sequences and then the “second generation” sequences is prepared and expressed as a protein.

Standard molecular biology techniques can be used to prepare and express altered antibody sequence. Preferably, the antibody encoded by the altered antibody sequences is one that retains one, some or all of the functional properties of the antibodies disclosed herein, respectively, produced by methods and with sequences provided herein, which functional properties include binding to a cancer cell antigen with a specific KD level or less and/or modulating immune stimulation and/or selectively binding to desired target cells such as for example, that express cancer associated antigen.

The functional properties of the altered antibodies can be assessed using standard assays available in the art and/or described herein. In some embodiments, mutations can be introduced randomly or selectively along all or part of an antibody coding sequence disclosed herein and the resulting modified antibodies can be screened for binding activity and/or other desired functional properties. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### Species Selectivity and Species Cross-Reactivity

According to certain embodiments of the present disclosure, the antibodies or antigen binding fragment thereof can bind to human cancer antigen but not to cancer antigen from other species. Alternatively, the antibodies or antigen binding fragment thereof, in certain embodiments, bind to human cancer antigen and to cancer antigen from one or more non-human species. For example, the antibodies or antigen binding fragment thereof can bind to human cancer antigen and can bind or not bind, as the case may be, to one or more of mouse, rat, guinea pig, hamster, gerbil, pig, cat, dog, rabbit, goat, sheep, cow, horse, camel, cynomologous, marmoset, rhesus or chimpanzee cancer antigen.

### Nucleic Acid Molecules Encoding Antibodies

Another aspect of the present disclosure pertains to nucleic acid molecules comprising reconstructed consensus nucleic acid sequences that encode the antibody polypeptide, described herein or antigen binding fragment thereof. Nucleic acids according to at least some embodiments of the present disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

### Identification of Target Antigens

### Screening Methods

Antibodies may be screened for binding affinity by methods known in the art. For example, gel-shift assays, Western blots, radiolabeled competition assay, co-fractionation by chromatography, co-precipitation, cross linking, ELISA, and the like may be used, which are described in, for example, Current Protocols in Molecular Biology (1999) John Wiley & Sons, NY, which is incorporated herein by reference in its entirety.

To initially screen for antibodies which bind to the desired epitope on an antigen (e.g., a cancer associated antigen), a routine cross-blocking assay such as that described in Antibodies, A Laboratory Manual, Cold Spring Harbor Laboratory, Ed Harlow and David Lane (1988), can be performed. Routine competitive binding assays may also be used, in which the unknown antibody is characterized by its ability to inhibit binding of antigen to an antigen specific antibody of the invention. Intact antigen, fragments thereof, or linear epitopes can be used. Epitope mapping is described in Champe et al., J. Biol. Chem. 270: 1388-1394 (1995).

The antibodies or antigen binding fragment thereof, described herein, may also be useful in preventing or treating cancer. The effectiveness of a candidate antibody or antigen binding fragment thereof in preventing or treating cancer metastasis may be screened using a human anmionic basement membrane invasion model as described in Filderman et al., Cancer Res 52: 36616, 1992. In addition, any of the animal model systems for metastasis of various types of cancers may also be used. Such model systems include, but are not limited to, those described in Wenger et al., Clin. Exp. Metastasis 19: 169 73, 2002; Yi et al., Cancer Res. 62: 917 23, 2002; Tsutsumi et al., Cancer Lett 169: 77-85, 2001; Tsingotjidou et al., Anticancer Res. 21: 971 8, 2001; Wakabayashi et al., Oncology 59: 75 80, 2000; Culp and Kogerman, Front Biosci. 3:D672 83, 1998; Runge et al., Invest Radiol. 32: 212 7; Shioda et al., J. Surg. Oneal. 64: 122 6, 1997; Ma et al., Invest Ophthalmol Vis Sci. 37: 2293 301, 1996; Kuruppu et al., J Gastroenterol Hepatol. 11: 26 32, 1996. In the presence of an effective antibody, cancer metastases may be prevented, or inhibited to result in fewer and/or smaller metastases.

The anti-tumor activity of a particular antibody, or combination of antibodies, or fragment thereof may be evaluated in vivo using a suitable animal model. For example, xenogenic lymphoma cancer models wherein human lymphoma cells are introduced into immune com-promised animals, such as nude or SCID mice. Efficacy may be predicted using assays which measure inhibition of tumor formation, tumor regression or metastasis, and the like.

In one variation of an in vitro assay, the present disclosure provides a method comprising the steps of (a) contacting an immobilized antigen with a candidate antibody and (b) detecting binding of the candidate antibody to the antigen. In an alternative embodiment, the candidate antibody is immobilized and binding of antigen is detected. Immobilization is accomplished using any of the methods well known in the art, including covalent bonding to a support, a bead, or a chromatographic resin, as well as non-covalent, high affinity interaction such as antibody binding, or use of streptavidin/biotin binding wherein the immobilized compound includes a biotin moiety. Detection of binding can be accomplished (i) using a radioactive label on the compound that is not immobilized, (ii) using a fluorescent label on the non-immobilized compound, (iii) using an antibody immunospecific for the non-immobilized compound, (iv) using a label on the non-immobilized compound that excites a fluorescent support to which the immobilized compound is attached, as well as other techniques well known and routinely practiced in the art.

Antibodies that modulate (e.g., increase, decrease, or block) the activity or expression of desired target may be identified by incubating a putative modulator with a cell expressing the desired target and determining the effect of the putative modulator on the activity or expression of the target. The selectivity of an antibody that modulates the activity of a target polypeptide or polynucleotide can be evaluated by comparing its effects on the target polypeptide or polynucleotide to its effect on other related compounds. Selective modulators may include, for example, antibodies and other proteins, peptides, or organic molecules which specifically bind to target polypeptides or to a nucleic acid encoding a target polypeptide. Modulators of target activity will be therapeutically useful in treatment of diseases and physiological conditions in which normal or aberrant activity of target polypeptide is involved. The target can be a for example, but not limited to a cancer associated antigen.

The invention also comprehends high throughput screening (HTS) assays to identify antibodies that interact with or inhibit biological activity (e.g., inhibit enzymatic activity, binding activity, etc.) of an antigen. HTS assays permit screening of large numbers of compounds in an efficient manner. Cell-based HTS systems are contemplated to investigate the interaction between antibodies and their target antigen and their binding partners. HTS assays are designed to identify “hits” or “lead compounds” having the desired property, from which modifications can be designed to improve the desired property. Chemical modification of the “hit” or “lead compound” is often based on an identifiable structure/activity relationship between the “hit” and target antigen.

Another aspect of the present invention is directed to methods of identifying antibodies which modulate (e.g., decrease) activity of a target antigen comprising contacting a target antigen with an antibody, and determining whether the antibody modifies activity of the antigen. The activity in the presence of the test antibody is compared to the activity in the absence of the test antibody. Where the activity of the sample containing the test antibody is lower than the activity in the sample lacking the test antibody, the antibody will have inhibited activity.

A variety of heterologous systems is available for functional expression of recombinant polypeptides that are well known to those skilled in the art. Such systems include bacteria (Strosberg, et al., Trends in Pharmacological Sciences (1992) 13:95-98), yeast (Pausch, Trends in Biotechnology (1997) 15:487-494), several kinds of insect cells (Vanden Broeck, Int. Rev. Cytology (1996) 164:189-268), amphibian cells (Jayawickreme et al., Current Opinion in Biotechnology (1997) 8: 629-634) and several mammalian cell lines (CHO, HEK293, COS, etc.; see Gerhardt, et al., Eur. J. Pharmacology (1997) 334:1-23). These examples do not preclude the use of other possible cell expression systems, including cell lines obtained from nematodes (PCT application WO 98/37177).

In one embodiment of the invention, methods of screening for antibodies which modulate the activity of target antigen comprise contacting antibodies with a target antigen polypeptide and assaying for the presence of a complex between the antibody and the target antigen. In such assays, the ligand is typically labeled. After suitable incubation, free ligand is separated from that present in bound form, and the amount of free or uncomplexed label is a measure of the ability of the particular antibody to bind to the target antigen.

The present disclosure encompasses the use of HTS to identify and characterize target antigens. A HTS can be protein arrays (e.g., antibody arrays, antibody microarrays, protein microarray). The array can comprise one or more antibodies or antigen binding fragment thereof, disclosed herein, immobilized on a solid support. Methods of production and use of such arrays are known well known in art (e.g., (Buessow et al., 1998, Lueking et al., 2003; Angenendt et al., 2002, 2003 a,b, 2004a, 2004b, 2006) In some embodiments, very small amounts (e.g., 1 to 500 μg) of antibody or antigen binding fragment thereof is immobilized. In some embodiments, there will be from 1 μg to 100 μg, from 1 μg to 50 μg, from 1 μg to 20 μg, from 3 μg to 100 μg, from 3 μg to 50 μg, from 3 μg to 20, from 5 μg to 100 μg, from 5 μg to 50 μg, from 5 μg to 20 μg of antibody present in a single sample. In one aspect, at least one of the samples in a plurality of samples will have from 1 μg to 100 μg, from 1 μg to 50 μg, from 1 μg to 20 μg, from 3 μg to 100 μg, from 3 μg to 50 μg, from 3 μg to 20, from 5 μg to 100 μg, from 5 μg to 50 μg, from 5 μg to 20 μg of antibody present. A solid support refers to an insoluble, functionalized material to which the antibodies can be reversibly attached, either directly or indirectly, allowing them to be separated from unwanted materials, for example, excess reagents, contaminants, and solvents. Examples of solid supports include, for example, functionalized polymeric materials, e.g., agarose, or its bead form Sepharose®, dextran, polystyrene and polypropylene, or mixtures thereof; compact discs comprising microfluidic channel structures; protein array chips; pipet tips; membranes, e.g., nitrocellulose or PVDF membranes; and microparticles, e.g., paramagnetic or non-paramagnetic beads. In some embodiments, an affinity medium will be bound to the solid support and the antibody will be indirectly attached to solid support via the affinity medium. In one aspect, the solid support comprises a protein A affinity medium or protein G affinity medium. A “protein A affinity medium” and a “protein G affinity medium” each refer to a solid phase onto which is bound a natural or synthetic protein comprising an Fc-binding domain of protein A or protein G, respectively, or a mutated variant or fragment of an Fc-binding domain of protein A or protein G, respectively, which variant or fragment retains the affinity for an Fc-portion of an antibody. Antibody arrays can be fabricated by the transfer of antibodies onto the solid surface in an organized high-density format followed by chemical immobilization. Representative techniques for fabrication of an array include photolithography, ink jet and contact printing, liquid dispensing and piezoelectrics. The patterns and dimensions of antibody arrays are to be determined by each specific application. The sizes of each antibody spot may be easily controlled by the users. Antibodies may be attached to various kinds of surfaces via diffusion, adsorption/absorption, or covalent cross-linking and affinity. Antibodies may be directly spotted onto a plain glass surface. To keep antibodies in a wet environment during the printing process, high percent glycerol (e.g., 30-40%) may be used in sample buffer and the spotting is carried out in a humidity-controlled environment.

The surface of a substrate may be modified to achieve better binding capacity. For example, the glass surface may be coated with a thin nitrocellulose membrane or poly-L-lysine such that antibodies can be passively adsorbed to the modified surface through non-specific interactions. Antibodies may be immobilized onto a support surface either by chemical ligation through a covalent bond or non-covalent binding. There are many known methods for covalently immobilizing antibodies onto a solid support. For example, MacBeath et al., (1999) J. Am. Chem. Soc. 121:7967-7968) use the Michael addition to link thiol-containing compounds to maleimide-derivatized glass slides to form a microarray of small molecules. See also, Lam & Renil (2002) Current Opin. Chemical Biol. 6:353-358. Depending upon, if the potential antigen is associated with a specific type of cancer, an antibody specific to a further biomarker may be included in the antibody array. Representative examples of biomarkers include, TROP/TNFRSF19, IL-1 sRI, uPAR, IL-10, VCAM-1 (CD106), IL-10 receptor-β, VE-cadherin, IL-13 receptor-α1, VEGF, IL-13 receptor-α2, VEGF R2 (KDR), IL-17, VEGF R3

The arrays can employ single-antibody (label-base) detection or 2-antibody (sandwich-based) detection. In some embodiments, an ELISA (also known as an antibody sandwich assay) may be performed following standard techniques as follows. Antibodies used as the capture antibodies for an antigen disposed on (e.g., coated onto) a solid support, which may then be washed at least once (e.g., with water and/or a buffer such as PBS-t), followed by a standard blocking buffer, and then at least one more wash. The solid support may then be brought into contact with the sample/biosample under conditions to allow antibody-antigen complexes to form (e.g., incubating from 1 hour to about 24 hours at a temperature from about 4° C. to about room temperature). As used herein, “biosample” and “sample” are used interchangeably and embrace both fluids (also referred to herein as fluid samples and biofluids) and tissue obtained from the subject. The term “biofluid” as used herein refers to a biological fluid sample such as blood samples, cerebral spinal fluid (CSF), urine and other liquids obtained from the subject, or a solubilized preparation of such fluids wherein the cell components have been lysed to release intra-cellular contents into a buffer or other liquid medium. The definition also includes samples that have been manipulated in any way after their procurement, such as by treatment with reagents, or enrichment for certain components, such as proteins or polynucleotides. The term “blood sample” embraces whole blood, plasma, and serum. Solid tissue samples include biopsy specimens and tissue cultures or cells derived therefrom, and the progeny thereof. A sample may comprise a single cell or more than a single cell. The biosample may also be a cultured population of cells derived from the subject human or animal. However, whenever the biosample comprises a population of cells, the method will first require that the constituents of the cells be solubilized by lysing the cells, and removing solid cell debris, thereby providing a solution of the biomarkers. Samples can be prepared by methods known in the art such as lysing, fractionation, purification, including affinity purification, FACS, laser capture micro-dissection or iospycnic centrifugation. The support may then be washed at least once (e.g., with a buffer such as PBS-t). To detect the complexation between the capture antibodies and the antigen that may be present in the sample, secondary or “detection” antibodies are applied to the solid support (e.g., diluted in blocking buffer) under conditions to allow complexation between the secondary antibodies and the respective biomarkers (e.g., at room temperature for at least one hour). The secondary antibodies are selected so as to bind a different epitope on the antigen than the capture antibody. The optimum concentrations of capture and detection antibodies are determined using standard techniques such as the “criss-cross” method of dilutions. The detection antibody may be conjugated, directly or indirectly, to a detectable label.

The term “detectable label” as used herein refers to labeling moieties known in the art. Said moiety may be, for example, a radiolabel (e.g., ³H, ¹²⁵I, ³⁵S, ¹⁴C, ³²P, etc.), detectable enzyme (e.g., horse radish peroxidase (HRP), alkaline phosphatase etc.), a dye (e.g., a fluorescent dye), a colorimetric label such as colloidal gold or colored glass or plastic (e.g., polystyrene, polypropylene, latex, etc.), beads, or any other moiety capable of generating a detectable signal such as a colorimetric, fluorescent, chemiluminescent or electrochemiluminescent (ECL) signal. The term “dye” as used herein refers to any reporter group whose presence can be detected by its light absorbing or light emitting properties. For example, Cy5 is a reactive water-soluble fluorescent dye of the cyanine dye family. Cy5 is fluorescent in the red region (about 650 to about 670 nm). It may be synthesized with reactive groups on either one or both of the nitrogen side chains so that they can be chemically linked to either nucleic acids or protein molecules. Labeling is done for visualization and quantification purposes. Cy5 is excited maximally at about 649 nm and emits maximally at about 670 nm, in the far red part of the spectrum; quantum yield is 0.28 (FW=792). Suitable fluorophores (chromes) for the probes of the disclosure may be selected from, but not intended to be limited to, fluorescein isothiocyanate (FITC, green), cyanine dyes Cy2, Cy3, Cy3.5, Cy5, Cy5.5 Cy7, Cy7.5 (ranging from green to near-infrared), Texas Red, and the like. Derivatives of these dyes for use in the embodiments of the disclosure may be, but are not limited to, Cy dyes (Amersham Bioscience), Alexa Fluors (Molecular Probes Inc.), HILYTE™ Fluors (AnaSpec), and DYLITE™ Fluors (Pierce, Inc). In some embodiments, the detectable label is a chromogenic label such as biotin, in which case the detection antibody-biotin conjugate is detected using Streptavidin/Horseradish Peroxidase (HRP) or the equivalent. The streptavidin may be diluted in an appropriate block and incubated for 30 minutes at room temperature. Other detectable labels suitable for use in the present invention include fluorescent labels and chemiluminescent labels.

The support may then be washed and the label (e.g., HRP enzymatic conjugate on the streptavidin) is detected using the following standard protocols such as a chromogenic system (the SIGMA FAST™ OPD system), a fluorescent system or a chemiluminescent system. The amounts of antigen present in the sample may then be read on an ELISA plate reader (e.g., SpectraMax 384 or the equivalent). The concentration of each of the antigens may then be back-calculated (e.g., by using the standard curve generated from purified antigens and multiplied by the dilution factor following standard curve fitting methods), and then compared to a control (generated from tissue samples obtained from healthy subjects).

In one embodiment, a biosample, e.g., a biofluid, is contacted with a system of reagents, well-known in the art, that can attach biotin moieties to some or all of the constituent components of the sample, and especially to the protein or peptide constituents thereof, including the biomarkers. Following this biotinylation step, the biotinylated biosample may then be contacted with the antibody array that contains an array of antibodies specific to each of the antigens.

After an adequate incubation period, readily selected to allow the binding of any antigen in the sample to its corresponding antibody of the array, the fluid sample is washed from the array. The array is then contacted with a biotin-binding polypeptide such as avidin or streptavidin, that has been conjugated with a detectable label (as described above in connection with the ELISA). Detection of the label on the array (relative to a control) will indicate which of the biomarkers captured by the respective antibody is present in the sample.

Regardless of the specific assay format, the biotin-label-based array methods are relatively advantageous from several standpoints. Biotin-label can be used as signal amplification. Biotin is the most common method for labeling protein and the label process can be highly efficient. Furthermore, biotin can be detected using fluorescence-streptavidin and, therefore, visualized via laser scanner, or HRP-streptavidin using chemiluminescence. Using biotin-label-based antibody arrays, most targeted proteins can be detected at pg/ml levels. The detection sensitivity of the present methods can be further enhanced by using 3-DNA detection technology or rolling circle amplification (Schweitzer et al., (2000) Proc. Natl. Acad. Sci. U.S.A. 97:10113-10119; Horie et al., (1996) Int. J. Hematol. 63:303-309).

As it relates to the present disclosure, the sample can be obtained from a subject having disease (e.g., cancer) and a healthy subject.

In some embodiments, protein arrays can be used where protein antigens with known identities are immobilized on a solid support as capture molecules and one seeks to determine whether the known antigens binds to a candidate antibody. The antigen can be labeled with a tag that allows detection or immunoprecipitation after capture by an immobilized antibody. Protein antigens can be obtained, for example, from a cancer patient or a cancer cell. A number of commercial protein arrays are available e.g., ProtoArray®, Kinex™, RayBio® Human RTK Phosphorylation Antibody Array. The antibody-antigen complexes can be obtained by methods known in the art (e.g., immunoprecipitation or Western blot). For reviews on Protein array and antibody array that can be of interest in this study, see Reymond Sutandy, et al., 2013; Liu, B. C.-S., et al. 2012; Haab B B, 2005.

In an exemplary immnuoprecipitation method, an antibody or antigen binding fragment thereof, described herein is added first to a sample comprising an antigen, and incubated to allow antigen-antibody complexes to form. Subsequently, the antigen-antibody complexes are or with protein A/G-coated beads to allow them to absorb the complexes. In a modified approach, the antibody or antigen binding fragment thereof is fused to a His tag or other tags (e.g., FLAG tag, Biotin Tag) by recombinant DNA techniques, and immunoprecipitated using an antibody to the tag (pull-down assay). The beads are then thoroughly washed, and the antigen is eluted from the beads by an acidic solution or SDS. The eluted sampled can be analyzed using Mass Spectrometry or SDS page to identify and confirm the antigen. Methods to analyze antibody-antigen complexes formed on a protein microarray and identify the antigen via mass spec are known.

In one aspect, the antibodies or antigen binding fragment thereof, disclosed herein, are contemplated as therapeutic antibodies for treatment of cancer. Accordingly, the antibodies or antigen binding fragment thereof, can be further screened in an antibody-dependent cell-mediated cytotocity (ADCC) assay and/or Complement-dependent cytotoxicity (CDC) assay. “ADCC activity” refers to the ability of an antibody to elicit an ADCC reaction. ADCC is a cell-mediated reaction in which antigen-nonspecific cytotoxic cells that express FcRs (e.g., natural killer (NK) cells, neutrophils, and macrophages) recognize antibody bound to the surface of a target cell and subsequently cause lysis of (e.g., “kill”) the target cell (e.g., cancer cell). The primary mediator cells are natural killer (NK) cells. NK cells express FcγRIII only, with FcγRIIIA being an activating receptor and FcγRIIIB an inhibiting one; monocytes express FcγRI, FcγRII and FcγRIII (Ravetch et al. (1991) Annu. Rev. Immunol., 9:457-92). ADCC activity can be assessed directly using an in vitro assay, e.g., a ⁵¹Cr release assay using peripheral blood mononuclear cells (PBMC) and/or NK effector cells as described in the Examples and Shields et al. (2001) J. Biol. Chem., 276:6591-6604, or another suitable method known in the art. ADCC activity may be expressed as a concentration of antibody at which the lysis of target cells is half-maximal. Accordingly, in some embodiments, the concentration of an antibody or antigen binding fragment thereof of the disclosure, at which the lysis level is the same as the half-maximal lysis level by the wild-type control, is at least 2—, 3-, 5-, 10-, 20-, 50-, 100-fold lower than the concentration of the wild-type control itself.

Additionally, in some embodiments, the antibody or antigen binding fragment thereof of the present disclosure may exhibit a higher maximal target cell lysis as compared to the wild-type control. For example, the maximal target cell lysis of an antibody or Fc fusion protein of the invention may be 10%, 15%, 20%, 25% or more higher than that of the wild-type control. “Complement dependent cytotoxicity” or “CDC” refer to the ability of a molecule to lyse a target (e.g. cancer cell) in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

### Epitope Mapping

The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., NY), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antigen-binding domain of an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues, which correspond to the specific amino acids with which the antibody interacts. See, e.g., Ehring (1999) Analytical Biochemistry 267(2):252-259; Engen and Smith (2001) Anal. Chem. 73:256A-265A. X-ray crystallography of the antigen/antibody complex may also be used for epitope mapping purposes.

The epitope on an antigen to which the antibody or antigen binding fragment, disclosed herein, bind may consist of a single contiguous sequence of 3 or more (e.g., 3, 4, 5, 6, 7, 8, 9, 10, 11, 12, 13, 14, 15, 16, 17, 18, 19, 20 or more) amino acids of the antigen. Alternatively, the epitope may consist of a plurality of non-contiguous amino acids (or amino acid sequences) of antigen.

### Antigens

In some embodiments, the systems and methods disclosed herein allow generation of reconstructed consensus sequences for antibodies or antigen biding fragment thereof that are directed to a cancer associated antigen. In some embodiments, the cancer associated antigen is a tumor antigen, e.g., a part of a tumor cell such as a protein or peptide expressed in a tumor cell which may be derived from the cytoplasm, the cell surface or the cell nucleus, in particular those which primarily occur intracellularly or as surface antigens of tumor cells. For example, tumor antigens include the carcinoembryonal antigen, α1-fetoprotein, isoferritin, and fetal sulphoglycoprotein, α2-H-ferroprotein and γ-fetoprotein. The term “cancer associated antigen” as used herein can be any type of cancer antigen that may be associated with a cancer as is known in the art and includes antigens found on the cell surface, including tumor cells, as well as soluble cancer antigens. Several cell surface antigens on tumors and normal cells have soluble counterparts. A cancer associated antigen can be a cell surface antigen or a soluble cancer antigen located in the tumor microenvironment or otherwise in close proximity to the tumor being treated. Such antigens include, but are not limited to those found on cancer-associated fibroblasts (CAFs), tumor endothelial cells (TEC) and tumor-associated macrophages (TAM). Examples of cancer-associated fibroblasts (CAFs) target antigens include but are not limited to: carbonic anhydrase IX (CAIX); fibroblast activation protein alpha (FAPα); and matrix metalloproteinases (MMPs) including MMP-2 and MMP-9. Examples of Tumor endothelial cell (TECs) target antigens include, but are not limited to vascular endothelial growth factor (VEGF) including VEGFR-1, 2, and 3; CD-105 (endoglin), tumor endothelia markers (TEMs) including TEM1 and TEM8; MMP-2; Survivin; and prostate-specific membrane antigen (PMSA). Examples of tumor associated macrophage antigens include, but are not limited to: CD105; MMP-9; VEGFR-1, 2, 3 and TEM8. In one embodiment, the cancer associated antibody specific for a cancer associated antigen may be specific for cancer antigens located on non-tumor cells, for example, VEGFR-2, MMPs, Survivin, TEM8 and PMSA. The cancer associated antigen may be an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, or a head and neck cancer antigen. A cancer antigen can also be a lymphoma antigen (e.g., non-Hodgkin's lymphoma or Hodgkin's lymphoma), a B-cell lymphoma cancer antigen, a leukemia antigen, a myeloma (e.g., multiple myeloma or plasma cell myeloma) antigen, an acute lymphoblastic leukemia antigen, a chronic myeloid leukemia antigen, or an acute myelogenous leukemia antigen. According to the present invention, a cancer associated antigen preferably comprises any antigen which is expressed in and optionally characteristic with respect to type and/or expression level for tumors or cancers as well as for tumor or cancer cells. In one embodiment, the term “tumor antigen” or “tumor-associated antigen” or “cancer antigen” or “cancer associated antigen” relates to proteins that are under normal conditions specifically expressed in a limited number of tissues and/or organs or in specific developmental stages, for example, the cancer associated antigen may be under normal conditions specifically expressed in stomach tissue, preferably in the gastric mucosa, in reproductive organs, e.g., in testis, in trophoblastic tissue, e.g., in placenta, or in germ line cells, and are expressed or aberrantly expressed in one or more tumor or cancer tissues. In this context, “a limited number” preferably means not more than 3, more preferably not more than 2. The cancer associated antigen in the context of the present invention include, for example, differentiation antigens, preferably cell type specific differentiation antigens, e.g., proteins that are under normal conditions specifically expressed in a certain cell type at a certain differentiation stage, cancer/testis antigens, e.g., proteins that are under normal conditions specifically expressed in testis and sometimes in placenta, and germ line specific antigens. Preferably, the cancer associated antigen or the aberrant expression of the cancer associated antigen identifies cancer cells. In the context of the present invention, the cancer associated antigen that is expressed by a cancer cell in a subject, e.g., a patient suffering from a cancer disease, is preferably a self-protein in said subject. In preferred embodiments, the cancer associated antigen in the context of the present invention is expressed under normal conditions specifically in a tissue or organ that is non-essential, e.g., tissues or organs which when damaged by the immune system do not lead to death of the subject, or in organs or structures of the body which are not or only hardly accessible by the immune system. A “cancer associated antigen”, as used herein can be any antigenic substance produced or overexpressed in tumor cells. It can, for example, trigger an immune response in the host. Alternatively, for purposes of this disclosure, cancer associated antigens can be proteins that are expressed by both healthy and tumor cells, but because they identify a certain tumor type, they can be a suitable therapeutic target. Non-limiting examples of the cancer associated antigen is CD19, CD20, CD30, CD33, CD38, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, or any combination thereof. In some embodiments, the cancer associated antigen is 1p19q, ABLI, AKT1, ALK, APC, AR, ATM, BRAF, BRCA1, BRCA2, cKIT, cMET, CSF1R, CTNNB1, EGFR, EGFRvIII, ER, ERBB2 (HER2), FGFR1, FGFR2, FLT3, GNA11, GNAQ, GNAS, HER2, HRAS, IDH1, IDH2, JAK2, KDR (VEGFR2), KRAS, MGMT, MGMT-Me, MLH1, MPL, NOTCHI, NRAS, PDGFRA, Pgp, PIK3CA, PR, PTEN, RET, RRM1, SMO, SPARC, TLE3, TOP2A, TOPO1, TP53, TS, TUBB3, VHL, CDH1, ERBB4, FBXW7, HNF1A, JAK3, NPM1, PTPN11, RB1, SMAD4, SMARCB1, STK1, MLH1, MSH2, MSH6, PMS2, microsatellite instability (MSI), ROS1, ERCC1, or any combination thereof. According to the invention, the terms “cancer associated antigen” “tumor antigen”, “tumor expressed antigen”, “cancer antigen” “cancer associated antigen” and “cancer expressed antigen” are equivalents and are used interchangeably herein.

### Fusion Proteins

In one aspect, provided herein is a fusion protein comprising an antibody or an antigen binding fragment, disclosed herein. In some embodiments, fusion protein comprises one or more antibody or antigen binding fragment thereof, disclosed herein, and an immunomodulator or toxin moiety. Methods of making antibody fusion proteins are known. Antibody fusion proteins comprising an interleukin-2 moiety are described by Boleti et al., Ann. Oneal. 6:945 (1995), Nicolet et al., Cancer Gene Ther. 2:161 (1995), Becker et al., Proc. Natl Acad. Sci. USA 93:7826 (1996), Hank et al., Clin. Cancer Res. 2:1951 (1996), and Hu et al., Cancer Res. 56:4998 (1996). In addition, Yang et al., Hum. Antibodies Hybridomas 6:129 (1995), describe a fusion protein that includes an F(ab′)2 fragment and a tumor necrosis factor alpha moiety.

### Chimeric Antigen Receptors

In one aspect, the disclosure herein, provides a chimeric antigen receptor comprising, an antigen binding fragment, disclosed herein, a transmembrane domain, and an intracellular signaling domain. The term “chimeric Antigen Receptor” (CAR), “artificial T cell receptor”, “chimeric T cell receptor”, or “chimeric immunoreceptor” as used herein refers to an engineered receptor, which grafts an arbitrary specificity onto an immune effector cell. CARs typically have an extracellular domain (ectodomain), which comprises an antigen-binding domain, a transmembrane domain, and an intracellular (endodomain) domain. The term “signaling domain” refers to the functional portion of a protein which acts by transmitting information within the cell to regulate cellular activity via defined signaling pathways by generating second messengers or functioning as effectors by responding to such messengers.

An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP12.

The term “zeta” or alternatively “zeta chain”, “CD3-zeta” or “TCR-zeta” is defined as the protein provided as GenBan Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to, an MHC class I molecule, BTLA and a Toll ligand receptor, as well as OX40, CD2, CD27, CD28, CD5, ICAM-1, LFA-1 (CD11a/CD18) and 4-1BB (CD137).

A costimulatory intracellular signaling domain can be derived from the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like.

The intracellular signaling domain can comprise the entire intracellular portion, or the entire native intracellular signaling domain, of the molecule from which it is derived, or a functional fragment thereof.

In another aspect, the antigen binding fragment comprises a humanized antibody or antibody fragment. In one embodiment, the antigen binding fragment comprises one or more (e.g., one, two, or all three) light chain complementary determining region 1 (CDR-L1), light chain complementary determining region 2 (CDR-L2), and light chain complementary determining region 3 (CDR-L3) of an antibody described herein, and one or more (e.g., one, two, or all three) heavy chain complementary determining region 1 (CDR-H1), heavy chain complementary determining region 2 (CDR-H2), and heavy chain complementary determining region 3 (CDR-H3) of an antibody described herein.

### Generation of Consensus Sequences Suitable for Treating Cancer

The disclosure provides systems and methods for generating polypeptide sequences for antibodies or antigen binding fragments thereof comprising reconstructed consensus polypeptide sequences suitable for treatment or prevention of a cancer, including, but not limited to, neoplasms, tumors, metastases, or any disease or disorder characterized by uncontrolled cell growth, by the administration of an antibody or antigen binding fragment thereof disclosed herein, to a patient in an amount effective to treat the patient.

In some embodiments, the cancer can be a carcinoma, a sarcoma, a lymphoma, a leukemia, germ cell tumor, a blastoma, or a melanoma. In some embodiments, the cancer can be a cancer from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In some embodiments, the cancer may be a neoplasm, malignant carcinoma, carcinoma, undifferentiated, giant and spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, adenocarcinoma; gastrinoma, cholangiocarcinoma, hepatocellular carcinoma, combined hepatocellular carcinoma and cholangiocarcinoma, trabecular adenocarcinoma, adenoid cystic carcinoma, adenocarcinoma in adenomatous polyp, adenocarcinoma, Familial adenomatous polyposis, solid carcinoma, carcinoid tumor, branchiolo-alveolar adenocarcinoma, papillary adenocarcinoma, chromophobe carcinoma, acidophil carcinoma, oxyphilic adenocarcinoma, basophil carcinoma, clear cell adenocarcinoma, granular cell carcinoma, follicular adenocarcinoma, papillary and follicular adenocarcinoma, nonencapsulating sclerosing carcinoma, adrenal cortical carcinoma, endometroid carcinoma, skin appendage carcinoma, apocrine adenocarcinoma, sebaceous adenocarcinoma, ceruminous adenocarcinoma, mucoepidermoid carcinoma, cystadenocarcinoma, papillary cystadenocarcinoma, papillary serous cystadenocarcinoma, mucinous cystadenocarcinoma, mucinous adenocarcinoma, signet ring cell carcinoma, infiltrating duct carcinoma, medullary carcinoma, lobular carcinoma, inflammatory carcinoma, paget's disease, mammary acinar cell carcinoma, adenosquamous carcinoma, adenocarcinoma w/squamous metaplasia, thymoma, ovarian stromal tumor, thecoma, granulosa cell tumor, androblastoma, sertoli cell carcinoma, leydig cell tumor, lipid cell tumor, paraganglioma, extra-mammary paraganglioma, pheochromocytoma, glomangiosarcoma, melanoma, Lentigo maligna, Lentigo maligna melanoma, Acral lentiginous melanoma, mucosal melanoma, nodular melanoma, polypoid melanoma, desmoplastic melanoma, skin cutaneous melanoma, amelanotic melanoma, superficial spreading melanoma, melanoma in giant pigmented nevus, epithelioid cell melanoma, blue nevus, sarcoma, fibrosarcoma, fibrous histiocytoma, myxosarcoma, liposarcoma, leiomyosarcoma, rhabdomyosarcoma, embryonal rhabdomyosarcoma, alveolar rhabdomyosarcoma, stromal sarcoma, mixed tumor, mullerian mixed tumor, nephroblastoma, hepatoblastoma, carcinosarcoma, mesenchymoma, brenner tumor, phyllodes tumor, synovial sarcoma, mesothelioma, dysgerminoma, embryonal carcinoma, teratoma, struma ovarii, choriocarcinoma, mesonephroma, hemangiosarcoma, hemangioendothelioma, kaposi's sarcoma, hemangiopericytoma, lymphangiosarcoma, osteosarcoma, juxtacortical osteosarcoma, chondrosarcoma, chondroblastoma, mesenchymal chondrosarcoma, giant cell tumor of bone, ewing's sarcoma, odontogenic tumor, ameloblastic odontosarcoma, ameloblastoma, ameloblastic fibrosarcoma, pinealoma, chordoma glioma, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, neurofibrosarcoma, neurilemmoma, granular cell tumor, malignant lymphoma, hodgkin's disease, hodgkin's, paragranuloma, lymphoma, small lymphocytic, malignant lymphoma, Diffuse large B-cell lymphoma, follicular lymphoma, mycosis fungoides, other specified non-hodgkin's lymphomas, histiocytosis, multiple myeloma, mast cell sarcoma, immunoproliferative small intestinal disease, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, myeloid sarcoma, or hairy cell leukemia. In some embodiments, the cancer is skin cutaneous melanoma.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a chronic immune condition, such as, but not limited to, a chronic infection or a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

### Definitions

The following definitions supplement those in the art and are directed to the current application and are not to be imputed to any related or unrelated case, e.g., to any commonly owned patent or application. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise. Furthermore, use of the term “including” as well as other forms, such as “include”, “includes,” and “included,” is not limiting.

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.”

The term “or” can be used conjunctively or disjunctively, unless the context specifically refers to a disjunctive use.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the invention, and vice versa. Furthermore, compositions of the invention can be used to achieve methods of the invention.

As used herein the term “consisting essentially of” refers to those elements required for a given embodiment. The term permits the presence of elements that do not materially affect the basic and novel or functional characteristic(s) of that embodiment of the invention.

As used herein the term “consisting of” refers to compositions, methods, and respective components thereof as described herein, which are exclusive of any element not recited in that description of the embodiment.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the inventions.

The terms “disease”, “disorder”, or “condition” are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

The term “in need thereof” when used in the context of a therapeutic or prophylactic treatment, means having a disease, being diagnosed with a disease, or being in need of preventing a disease, e.g., for one at risk of developing the disease. Thus, a subject in need thereof can be a subject in need of treating or preventing a disease.

As used herein, the term “administering,” refers to the placement of a compound (e.g., an antibody or antigen binding fragment thereof as disclosed herein) into a subject by a method or route that results in at least partial delivery of the agent at a desired site. Pharmaceutical compositions comprising an antibody or antigen binding fragment thereof, disclosed herein can be administered by any appropriate route which results in an effective treatment in the subject, including but not limited to intravenous, intraarterial, injection or infusion directly into a tissue parenchyma, etc. Where necessary or desired, administration can include, for example, intracerebroventricular (“icv”) administration, intranasal administration, intracranial administration, intracelial administration, intracerebellar administration, or intrathecal administration.

As used herein, a “subject”, “patient”, “individual” and like terms are used interchangeably and refers to a vertebrate, a mammal, a primate, or a human. Mammals include, without limitation, humans, primates, rodents, wild or domesticated animals, including feral animals, farm animals, sport animals, and pets. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, and canine species, e.g., dog, fox, wolf, avian species, e.g., chicken, emu, ostrich, and fish, e.g., trout, catfish and salmon. The terms, “individual,” “patient” and “subject” are used interchangeably herein. A subject can be male or female.

In some embodiments, the subject is a mammal. The mammal can be a human, non-human primate, mouse, rat, dog, cat, horse, or cow, but is not limited to these examples. Mammals other than humans can be advantageously used as subjects that represent animal models of conditions or disorders associated with uncontrolled cell growth (e.g., a cancer). Non-limiting examples include murine tumor models. In addition, the compositions and methods described herein can be used to treat domesticated animals and/or pets. A subject can be one who has been previously diagnosed with or identified as suffering from a cancer. A subject can be one who is diagnosed and currently being treated for, or seeking treatment, monitoring, adjustment or modification of an existing therapeutic treatment, or is at a risk of developing a given disorder (e.g., cancer).

A “cytotoxic agent” refers to an agent that has a cytotoxic and/or cytostatic effect on a cell. A “cytotoxic effect” refers to the depletion, elimination and/or the killing of a target cell(s). A “cytostatic effect” refers to the inhibition of cell proliferation.

As used herein, the terms “protein”, “peptide” and “polypeptide” are used interchangeably to designate a series of amino acid residues connected to each other by peptide bonds between the alpha-amino and carboxy groups of adjacent residues. The terms “protein”, “peptide” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein”, “peptide” and “polypeptide” are used interchangeably herein when referring to a gene product and fragments thereof.

An “antibody”, as used herein refers to an immunoglobulin molecule capable of specific binding to a target, (e.g., cancer associated antigen), through at least one antigen recognition site, located in the variable region of the immunoglobulin molecule. As used herein, the term encompasses not only intact antibodies, but also fragments thereof (such as Fab, Fab′, F(ab′)2, Fv), single chain (ScFv), mutants thereof, fusion proteins comprising an antibody portion, and any other modified configuration of the immunoglobulin molecule that comprises an antigen recognition site. An antibody includes an antibody of any class, such as IgG, IgA, IgD, IgE or IgM (or sub-class thereof), and the antibody need not be of any particular class.

As used herein, “monoclonal antibody” refers to an antibody obtained from a population of substantially homogeneous antibodies, e.g., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature, 256:495, or may be made by recombinant DNA methods. The monoclonal antibodies may also be isolated from phage libraries generated using the techniques described in McCafferty et al., 1990, Nature, 348:552-554, for example.

As used herein, “humanized” antibodies refer to forms of non-human (e.g. murine) antibodies that are specific chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Other forms of humanized antibodies have one or more CDRs (one, two, three, four, five, six) which are altered with respect to the original antibody, which are also termed one or more CDRs “derived from” one or more CDRs from the original antibody.

As used herein, an “isolated antibody” is one that has been separated and/or recovered from a component of its natural environment. Contaminant components of its natural environment are materials that would interfere with diagnostic or therapeutic uses of the antibody, and may include enzymes, hormones, and other proteinaceous or non-proteinaceous components. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity as shown by SDS-PAGE under reducing or non-reducing conditions and using Coomassie blue or, preferably, silver staining. Isolated antibody includes the antibody in situ within recombinant cells since at least one component of the antibody's natural environment will not be present. Ordinarily, however, isolated antibody will be prepared by at least one purification step.

As used herein, the term “Complementarity Determining Regions” (CDRs, e.g., CDR1, CDR2, and CDR3) refers to the amino acid residues of an antibody variable domain the presence of which are necessary for antigen binding. Each variable domain typically has three CDR regions identified as CDR1, CDR2 and CDR3. The CDRs of variable heavy chain can be CDR-H1, CDR-H2 and CDR-H3. The CDRs of variable light chain can be CDR-L1, CDR-L2 and CDRL3. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987)). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th ed. (1991)). Thus, the HVs may be comprised within the corresponding CDRs and references herein to the “hypervariable loops” of VH and VL domains should be interpreted as also encompassing the corresponding CDRs, and vice versa, unless otherwise indicated. The more highly conserved regions of variable domains are called the framework region (FR), as defined below. The variable domains of native heavy and light chains each comprise four FRs (FR1, FR2, FR3 and FR4, respectively), largely adopting a [beta]-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227: 799-817 (1992)); Tramontano et al., J. Mol. Biol, 215: 175-182 (1990)). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (e.g., Kabat et al. Sequences of Proteins of Immunological Interest, (5th ed., 1991, National Institutes of Health, Bethesda Md.)); and (2) an approach based on crystallographic studies of antigen-antibody complexes (Allazikani et al (1997) J. Molec. Biol. 273:927-948)). A CDR may refer to CDRs defined by either approach or by a combination of both approaches.

A “constant region” of an antibody refers to the constant region of the antibody light chain or the constant region of the antibody heavy chain, either alone or in combination. The constant region does not vary with respect to antigen specificity.

As used herein, the term “heavy chain region” includes amino acid sequences derived from the constant domains of an immunoglobulin heavy chain. A polypeptide comprising a heavy chain region comprises at least one of: a CH1 domain, a hinge (e.g., upper, middle, and/or lower hinge region) domain, a CH2 domain, a CH3 domain, or a variant or fragment thereof. In an embodiment, an antibody or an antigen binding fragment thereof may comprise the Fc region of an immunoglobulin heavy chain (e.g., a hinge portion, a CH2 domain, and a CH3 domain). In another embodiment, an antibody or an antigen binding fragment thereof lacks at least a region of a constant domain (e.g., all or part of a CH2 domain). In certain embodiments, at least one, and preferably all, of the constant domains are derived from a human immunoglobulin heavy chain. For example, in one preferred embodiment, the heavy chain region comprises a fully human hinge domain. In other preferred embodiments, the heavy chain region comprising a fully human Fc region (e.g., hinge, CH2 and CH3 domain sequences from a human immunoglobulin). In certain embodiments, the constituent constant domains of the heavy chain region are from different immunoglobulin molecules. For example, a heavy chain region of a polypeptide may comprise a domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. As set forth above, it will be understood by one of ordinary skill in the art that the constant domains of the heavy chain region may be modified such that they vary in amino acid sequence from the naturally occurring (wild-type) immunoglobulin molecule. That is, the polypeptides of the invention disclosed herein may comprise alterations or modifications to one or more of the heavy chain constant domains (CH1, hinge, CH2 or CH3) and/or to the light chain constant domain (CL). Exemplary modifications include additions, deletions or substitutions of one or more amino acids in one or more domains.

As used herein, the term “hinge region” includes the region of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al. J. Immunol. 1998 161:4083).

As used herein, the term “Fv” is the minimum antibody fragment that contains a complete antigen-recognition and -binding site. This fragment consists of a dimer of one heavy- and one light-chain variable region domain in tight, non-covalent association.

From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Framework” or FR residues are those variable domain residues other than the hypervariable region residues.

“Polyucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from reconstructed immunoglobulin consensus sequences, disclosed herein. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human immunoglobulin VH and VL sequences, may not naturally exist within the human antibody germline repertoire in vivo.

“Isolated nucleic acid”, as used herein, is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man. The term “polypeptide” is used in its conventional meaning, e.g., as a sequence of amino acids.

In the context of an antibody or antigen-binding fragment thereof, the term “specificity” or “specific for” refers to the number of different types of antigens or antigenic determinants to which a particular antibody or antigen-binding fragment thereof can bind. The specificity of an antibody or antigen-binding fragment or portion thereof can be determined based on affinity and/or avidity. The affinity, represented by the equilibrium constant for the dissociation (K_{D}) of an antigen with an antigen-binding protein, is a measure for the binding strength between an antigenic determinant and an antigen-binding site on the antigen-binding protein: the lesser the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest. Accordingly, an antibody or antigen-binding fragment thereof as defined herein is said to be “specific for” a first target or antigen compared to a second target or antigen when it binds to the first antigen with an affinity (as described above, and suitably expressed, for example as a K_{D} value) that is at least 50 times, such as at least 100 times, and preferably at least 1000 times, and up to 10,000 times or more better than the affinity with which said amino acid sequence or polypeptide binds to another target or polypeptide. Preferably, when an antibody or antigen-binding fragment thereof is “specific for” a target or antigen, compared to another target or antigen, it can bind the target or antigen, but does not bind the other target or antigen.

However, as understood by one of ordinary skill in the art, in some embodiments, where a binding site on a target is shared or partially shared by multiple, different ligands, an antibody or antigen binding fragment thereof can specifically bind to a target, such as cancer associated antigen, and have the functional effect of, for example, inhibiting/preventing tumor progression.

Avidity is the measure of the strength of binding between an antigen-binding molecule and the pertinent antigen. Avidity is related to both the affinity between an antigenic determinant and its antigen binding site on the antigen-binding molecule, and the number of pertinent binding sites present on the antigen-binding molecule. Typically, antigen-binding proteins will bind to their cognate or specific antigen with a dissociation constant (K_{D} of 10⁻⁵ to 10⁻¹² moles/liter or less, and preferably 10⁻⁷ to 10⁻¹² moles/liter or less and more preferably 10⁻⁸ to 10⁻¹² moles/liter (e.g. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 105 M (10000 nM). The stronger an interaction is, the lower is its K_{D}. Preferably, a binding site on an anti-LAP antibody or antigen-binding fragment thereof described herein will bind with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

The term “fusion protein” as used herein refers to a polypeptide that comprises an amino acid sequence of an antibody or fragment thereof and an amino acid sequence of a heterologous polypeptide (e.g., an unrelated polypeptide).

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

### Digital Processing Device

In some embodiments, the systems, devices, platforms, media, methods and applications described herein include a digital processing device, a processor, or use of the same. For example, in some embodiments, the digital processing device is part of a system for generating reconstructed consensus sequences described herein. In some embodiments, the system comprises a digital processing device. In some embodiments, the system is a computing system. In further embodiments, the digital processing device includes one or more processors or hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device. In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In some embodiments, the non-volatile memory comprises magnetoresistive random-access memory (MRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a subject. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In some embodiments, the display is E-paper or E ink. In other embodiments, the display is a video projector. In still further embodiments, the display is a combination of devices such as those disclosed herein.

In some embodiments, the digital processing device includes an input device to receive information from a subject. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, media, methods and applications described herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, media, methods and applications described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device such as a smartphone. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g. not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of barcode, route, parcel, subject, or network information. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

### Exemplary Methods

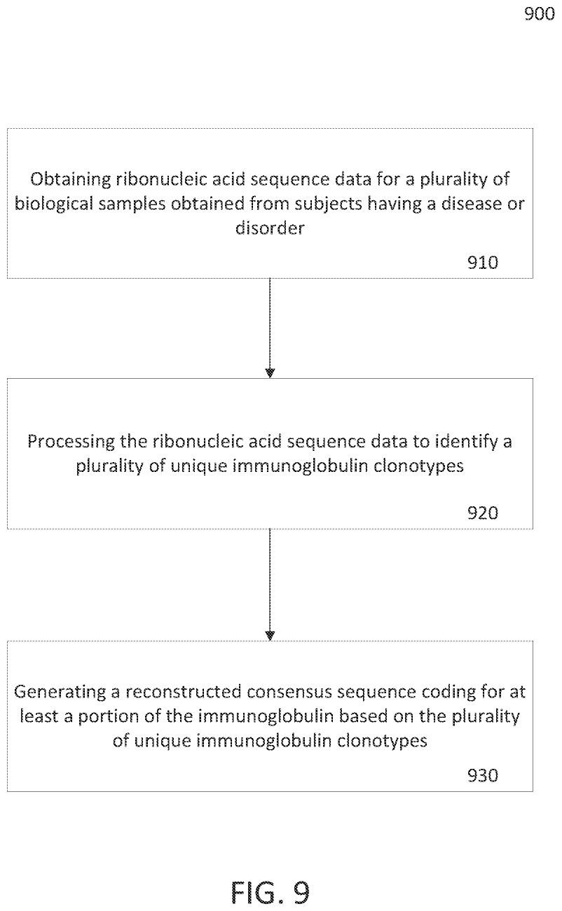

FIG. 9 discloses an exemplary method 900 of generating a reconstructed consensus sequence according to an embodiment of the disclosure. The method 900 can begin by obtaining ribonucleic acid sequence data for a plurality of biological samples obtained from subjects having a disease or disorder, such as cancer (step 910). The ribonucleic acid sequence data may then be processed to identify a plurality of unique immunoglobulin clonotypes (step 920). A reconstructed consensus sequence is then generated that codes for at least a portion of an immunoglobulin based on the plurality of unique immunoglobulin clonotypes (step 930).

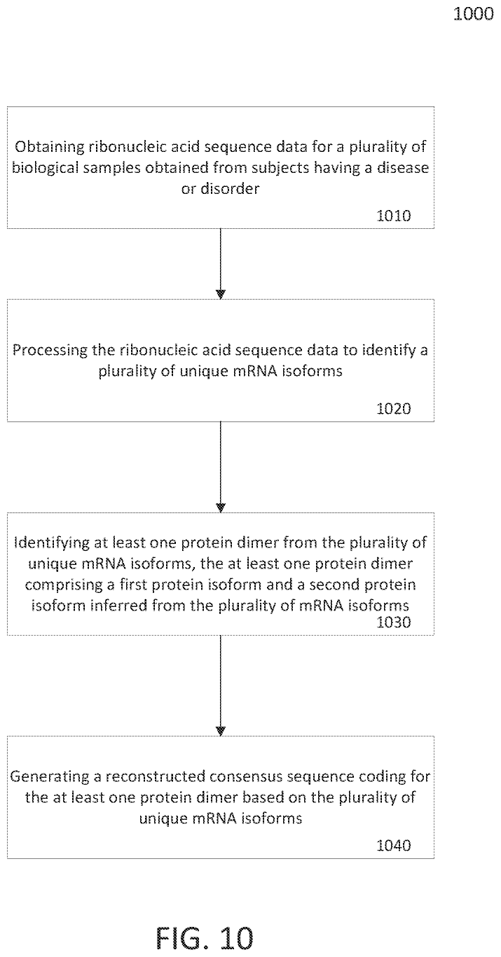

FIG. 10 discloses an exemplary method 1000 of identifying a protein dimer associated with a disease or disorder from mRNA sequencing data according to an embodiment of the disclosure. The method 1000 comprises obtaining ribonucleic acid sequence data for a plurality of biological samples obtained from subjects having the disease or disorder (step 1010). Ribonucleic acid may be derived from patient tissues that have experienced an acute immune response, such as from cancer, autoimmune disease, or an infectious disease, for example. The ribonucleic acid sequence data may then be processed to identify a plurality of unique mRNA transcripts (step 1020). Based on the plurality of unique mRNA transcripts, at least one protein dimer can be identified, wherein the at least one protein dimer comprises a first protein isoform and a second protein isoform inferred from the plurality of mRNA isoforms (step 1030). A reconstructed consensus sequence coding for the at least one protein dimer may then be generated (step 1040).

Processing ribonucleic acid sequence data (step 1020) may be performed in a variety of ways. In one embodiment, consider two genes, A and B. Gene A is capable of generating multiple mRNA isoforms (a₁, a₂, . . . aᵢ) of unknown sequence, encoding different protein isoforms, and Gene B is capable of generating multiple mRNA isoforms (b₁, b₂, . . . bᵢ) of unknown sequence, encoding different protein isoforms. Expressed copies of both Genes A and B may be present in the ribonucleic acid sequence data, which may be a bulk RNA sequencing dataset D containing short reads. D may be further filtered using a transcriptomic-referenced genomic aligner or similar substitutes such as pseudo-alignment to remove those short reads in D which have a high likelihood of having arising from genomic loci far away from the coding regions of A and B; this smaller set of reads can be referred to as D′. In one embodiment, ribonucleic acid sequence data sequence reads which align or pseudoalign to half a read length, one read length, two read lengths, or more away from a locus known to code for an mRNA isoform in a protein isomer are discarded.

In one embodiment, the most probable mRNA isoforms for genes A and B contained within D′ may be determined by assembling the short reads in D′, using (e.g.) de Bruijn graph assembly or an equivalent method such as Overlap-Layout-Consensus assembly, resulting in a set of mRNA isoforms from Gene A (a*₁, a*₂, . . . a*ᵢ) and Gene B (b*₁, b*₂, . . . b*ᵢ). In one embodiment, only those ribonucleic acid sequence data sequence reads which align within half a read length, one read length, two read lengths, or are inside a genomic locus known to code for an mRNA isoform in a protein isomer are assembled in silico into isoform sequences using (e.g.) de Bruijn graph assembly, or an equivalent method such as Overlap-Layout-Consensus assembly.

Identifying at least one protein dimer (step 1030) can be performed in a variety of ways. In one embodiment, once the set of mRNA isoforms from Gene A and Gene B have been assembled, the expression levels of each inferred mRNA isoform may be determined based on D′ using a gene expression quantification method known in the art. The expression level estimates may then be analyzed for each isoform in A and B to infer at least one protein dimer (a*ᵢ, b*ⱼ) that may form in vivo, the at least one protein dimer (a*ᵢ, b*) comprising a protein isoform of A (a*ᵢ) and a protein isoform of B (b*ⱼ). In one embodiment, pairing may be determined by calculating a score. In some embodiments, the score is a clonal ratio of the most abundant to the second most abundant isoform for each of Gene A and Gene B. In other embodiments, the score is a dominance score, which may be determined by calculating the Berger-Parker dominance index for each isoform in A and B, and then calculating a dominance score as the geometric mean of these indices. These measures may be used to identify at least one protein dimer (a*ᵢ, b*ⱼ) that is particularly dominant within a sample.

Protein dimers according to the disclosure can comprise any variety or combination of protein isomers, dimers, trimers, multi-mers, and the like. For example, a protein dimer may comprise two associated protein dimers, such as a complete antibody molecule comprising two heavy chains and two light chains. In some embodiments, a protein dimer may comprise a combination of a protein monomer and another protein dimer. Various embodiments and combinations of protein isoforms are considered within the scope of the disclosure.

In one embodiment, in vitro techniques are used to produce synthetic expression vectors capable of producing the pair of mRNA isoforms which are most highly expressed in the ribonucleic acid sequence data. In another embodiment, synthetic expression vectors are transfected into a transfection competent cell line, the cells are cultivated, and synthetic polypeptides comprising a protein dimer are expressed and purified.

Protein dimers inferred using the method 1000 may be experimentally validated to determine whether the protein dimer is useful for treating a disease or disorder. In one embodiment, in vitro techniques are used to validate. For example, two expression vectors can be generated that are capable of guiding the expression of the inferred protein isoforms a*ᵢ and b*ⱼ when transfected into a plurality of cells, such as a human cell line (e.g, HEK293 cells). The plurality of cells may then be transfected with the two expression vectors and cultivated. In vitro techniques may then be used to detect the presence of the hypothesized protein dimer (a*ᵢ, b*ⱼ) in the culture supernatant, and proteomics techniques may then be used to characterize the interactors of the inferred protein dimer (a*ᵢ, b*ⱼ) based on the data generated. Both in vivo and in vitro experiments aimed at assessing the viability of the dimer may be performed, and potential therapeutic applications of the inferred protein dimer (a*ᵢ, b*ⱼ) can be determined. Additional in vivo experiments may be performed to assess the therapeutic applications.

In one embodiment, in vitro proteomics techniques are used to characterize interactions of the resulting protein dimer, including but not limited to the identity of the target that the protein dimer binds, or the binding disassociation constant K_{d}, or an IC50 concentration at which the protein dimer attains 50% effectiveness in neutralizing viral infection. In one embodiment, experimentally derived knowledge of the protein dimer's in vitro biological interaction characteristics are used to hypothesize and perform in vivo testing of the protein dimer's usefulness as an active ingredient in a pharmaceutical composition or medicament for the therapeutic treatment of a disease.

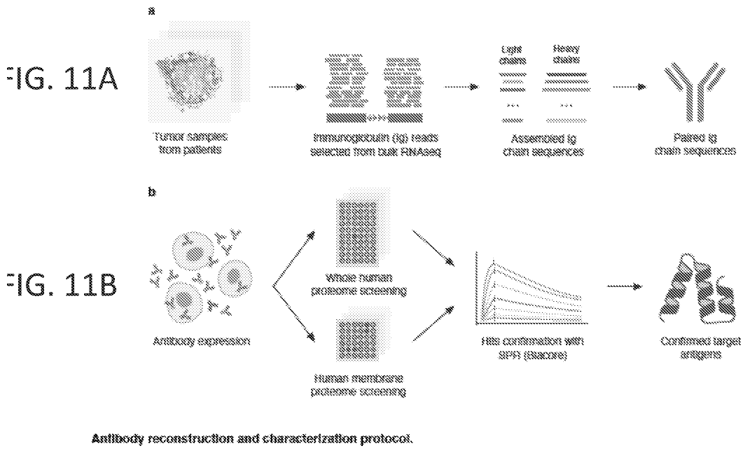

Methods according to the disclosure, such as the method 1000, have many applications, including for treating cancer, autoimmune disease, and infectious diseases. For example, the method 1000 may be applied to identify cancer-associated antibodies, which are protein dimers formed from immunoglobulin heavy chains and light chains. In this example, consider that Gene A is the IGH locus encoding the immunoglobulin heavy chain and Gene B is the IGK or IGL locus encoding the immunoglobulin light chain. These loci produce vast numbers of novel protein isoforms because of alternative splicing, class switching, somatic recombination, and somatic hypermutation. In this embodiment, the inferred protein dimer (a*ᵢ, b*ⱼ) is part of an immunoglobulin. Thus, using the method 1000 on bulk RNA sequencing data from cancer patients, the protein dimers identified will be immunoglobulins associated with, and thus likely binders of, that cancer, and may be used to treat that cancer, as illustrated in FIGS. 11A-B, and further described in the Examples below.

In another example, Gene A is the TRA locus encoding the T cell receptor alpha chain and B is the TRB locus encoding the T cell receptor beta chain. These loci produce vast numbers of novel protein isoforms due to alternative splicing and somatic recombination. In this embodiment, the inferred protein dimer (a*i, b*j) is part of a T cell receptor.

In another example, Genes A and B could be genes of the complement system. These loci produce novel protein isoforms due to alternative splicing. In this embodiment, the inferred protein dimer (a*i, b*j) may be a novel member of the complement cascade.

### Examples

Provided below are exemplary methods for in silico reconstruction of consensus sequences of cancer associated antibodies. Also described herein are computational analytical approaches for estimation of immunoglobulin repertoire diversity and the identification of clonal rearranged immunoglobulin CDR3 sequences present in the repertoire. The approaches are contemplated for the reconstruction of complete consensus sequences of the variable heavy chain, variable light chain and the respective CDR3 of said immunoglobulins. Also described herein are techniques for expressing and individually testing reconstructed consensus sequences, as well as identifying their target antigens and binding potential.

### Overview

Transcripts encoding immunoglobulin light and heavy chains are often detected in solid tumors across different cancer types, but their functional relevance remains unclear. Certain characteristics of the intratumoral Ig repertoire (e.g., transcripts abundance, clonality and number of detectable somatic mutations) have been associated with favorable clinical outcomes, such as longer overall survival and response to immune checkpoint inhibitors. Moreover, the presence of intratumoral plasma cells and ectopic germinal centers, which are key components of the antibody selection and production machinery, has been associated with longer overall survival and immunotherapy response. Despite these observations, the contribution of intratumoral Ig to immune responses against cancer remains largely unknown.

The main obstacle to the functional characterization of intratumoral Ig is our limited understanding of their target antigens. Previous studies have demonstrated that sequences of individual Ig chains can be reconstructed using bioinformatics methods from bulk RNA sequencing data (RNA-Seq) generated by large-scale cancer genomics efforts. Compared to specialized B-cell receptor (BCR) sequencing or single-cell sequencing, bulk RNA-seq has the significant advantage of being readily available for thousands of clinically annotated tumor samples. However, previous studies have been limited to in-silico analysis: they did not attempt to pair heavy and light Ig chains, nor to express the resulting sequences as complete antibodies, two key steps required to experimentally identify their target antigens.

In the below Examples we assembled and paired thousands of intratumoral Ig chains using legacy bulk RNA-Seq data from TCGA, one of the most comprehensive genomic studies of human cancer to date (as shown in FIGS. 11A-B). We individually performed gene synthesis, mammalian expression, and purification for 283 pairs of Ig chains, obtaining high quality, fully human recombinant 43 antibodies in most cases. We then individually screened each antibody against two large collections of recombinant proteins, covering the vast majority of the human proteome, in order to obtain the most likely binding targets. In selected cases, we confirmed binding using surface plasmon resonance (SPR) in order to characterize the binding kinetics. Our results show that fully functional antibodies can be obtained from legacy bulk tumour RNA-Seq, without the need for specialized BCR or single-cell sequencing. Using this approach, we were able to identify the target antigens of several high-affinity Ig expressed in human tumors.

Despite the striking correlation between expression of Ig transcripts and favourable clinical outcomes in human tumors, the functional role of intratumoral Ig remains substantially unknown. In the below Exapmles, we demonstrated for the first time that in-silico pairing of intratumoral Ig can be used to obtain fully functional antibodies from legacy tumor RNA sequencing data. Moreover, we show that it is possible to use high-throughput proteomics techniques to identify their target antigens, characterize their binding kinetics, and map the corresponding epitopes. These steps are crucial to enable further functional characterization studies. Interestingly, we have demonstrated that highly clonal intratumoral Ig are selected to bind not only cancer-specific antigens (NY-ESO-1, MAGEA3, GAGE2A, DLL3), but also wild-type proteins expressed in the tumor microenvironment (ANXA1, TGFBI, C4BPB). Despite being directed against non-mutated self-antigens, these Ig bind to their target with very high affinity, similarly to antibodies obtained by immunizing a different species with the same antigen. The importance of this observation is twofold: on one hand, it highlights the extent to which peripheral tolerance might be compromised during immune responses in cancer-affected tissues; on the other hand, it suggests that high-affinity fully human antibodies against human proteins can be obtained by sequencing Ig transcripts expressed in tissues affected by chronic inflammation.

While we have not identified the target antigens for many of the antibodies produced, it should be noted that our antigen screening only considered non-mutated proteins. The remaining orphan antibodies could be explained by errors in our in-silico pairing method. Alternatively, it is possible that some of these orphan antibodies bind to antigens for which we could not screen in this study, including neoantigens specific for particular patients, or non-protein antigens such as glycan. Advancements in antigen screening methods might in the future allow the deorphaning of additional candidates. Despite these limitations, this study generated the largest collection of individually screened fully human intratumoral Ig generated so far, paves the way to improve our understanding of their functional role during anti-tumor immune responses, and suggests a novel way to extract immunological insights from legacy RNA sequencing data.

FIG. 11A depicts steps of a computational workflow which starts from raw RNA sequencing data of tumor samples as the input, removes the reads mapped to non-Ig transcripts, reconstructs the Ig chain sequences, and outputs the paired sequences for which both chains satisfy a dominance threshold, as described in further detail in Examples 1-9 below. FIG. 11B depicts steps of an experimental workflow aimed at expressing the reconstructed Ig as recombinant antibodies, screening for their target antigens using two different human protein libraries and confirming the result using surface plasmon resonance (SPR), as described in further detail in Examples 10-13 below.

### Example 1: Estimation of the Immunoglobulin Repertoire Diversity

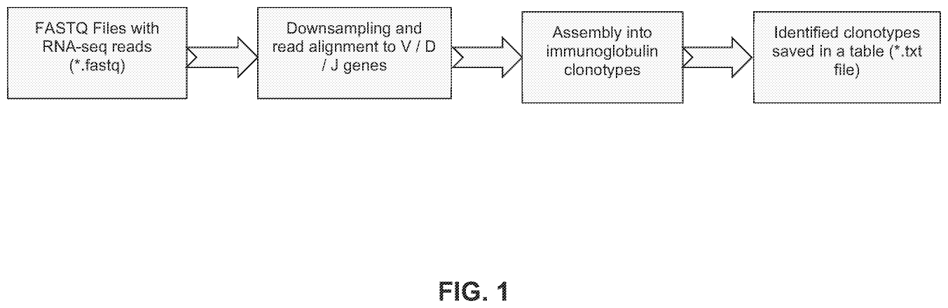

RNA-seq FASTQ files for 473 TCGA Skin Cutaneous Melanoma (SKCM) patients collected by TCGA consortium (The Cancer Genome Atlas, NCI &NHGRI) were recorded and analysed. RNA-seq samples (n=473) were aligned to reference V, D and J genes of immunoglobulins in order to identify the repertoire present in the samples. Then, identical CDR3 sequences were identified and grouped in clonotypes. The information was exported into a tab-delimited and understandable text file (FIG. 1). From the initial 473 samples, 178 samples were eliminated for which there were no reads aligning to immunoglobulin heavy chain genes or the number of reads was lower than the downsampling threshold and an additional 25 samples corresponding to lymph nodes. In total, the information on immunoglobulin (Ig) diversity from 270 melanoma samples was collected and analysed.

VDJtools were used to filter out non-functional (non-coding) clonotypes and to compute basic diversity statistics. Non-functional clonotypes were identified as those containing a stop codon or frameshift in their receptor sequence. The diversity of the Ig repertoire was based on the effective number of species which is calculated as the exponent of the Shannon-Wiener Entropy index such that a community of S species with species frequencies p1, . . . pi, . . . ps, then the diversity (D) is the exponent of the Shannon-Wiener Entropy index (H) given by:

$$D = exp ( H ) = exp ( - \text{?} {p}_{i} ln {p}_{i} )$$

$$\text{?} \text{indicates text missing or illegible when filed}$$

### Example 2: Identification of Clonal Immunoglobulin Sequences

The top 50 patients (highly clonal patients) were chosen to investigate their immunoglobulin sequences in more detail. Manual curation of immunoglobulin predictions and corresponding read alignments led to the selection of 14 patients for further alignment investigation. The table 8 below shows the clinical and clonality information for selected patients:

TABLE 8

| | OS survival | | |
|---|---|---|---|
| | time | | |
| sample_id | (months) | Status | Eff nr species |
| 1 | 20.76 | alive | 1.551084969 |
| 2 | 111.01 | dead | 1.827143864 |
| 3 | 180.26 | alive | 2.004177897 |
| 4 | 170.07 | alive | 2.044512649 |
| 5 | 62.02 | alive | 2.26857309 |
| 6 | 148.92 | alive | 5.616622449 |
| 7 | 176.41 | dead | 6.172934019 |
| 8 | 19.38 | alive | 6.880520052 |
| 9 | 92.9 | alive | 7.563366072 |
| 10 | 133.71 | alive | 7.834202032 |
| 11 | 135.64 | alive | 8.488138592 |
| 12 | 241.2 | alive | 9.05109639 |
| 13 | 160.87 | alive | 9.384014312 |
| 14 | 21.19 | alive | 10.11978853 |

### Example 3: Alignment and Assembly of V D J Sequences

Alignments were performed against the immunoglobulin segments identified by the first alignment step for viewing the results, allowing the exploration of the frequency distribution of sequence mismatches along the V, D, J gene segments and in particular in the CDR3 region length statistics. This alignment step was useful for summarizing repertoires, as well as offering a detailed view of rearrangements and region alignments for individual query sequences. More details about the alignment and assembly methodology are given in the Example 5 below.

In brief, the identified segments by first alignment step from IMGT were first provided using the reference files provided in the BraCeR tool. The heavy D segment and light V-J junction sequences were then reconstructed using an in-house built assembler (see Example 5 for detailed description). A FASTA file with corrected heavy D and light V-J junction sequences was generated for each sample. In addition to the assembled FASTA files germline FASTA files using IgBLAST v1.9.0 and IMGT database were also generated. The somatic FASTA sequence was inputed to IgBLAST and to obtain the closest segment ids for the heavy and light chain. Then, the germline FASTA were generated by merging corresponding segment sequences from the IMGT database. The final assembled FASTA sequences served as ‘reference’ sequences for the alignment and visualisation steps described below. All final ‘reconstructed’ nucleotide and amino acid consensus sequences are provided herein (Tables 1-4).

### Quality-Control and Visual Confirmation of Alignments

Using the reference files generated from the assembly step, the FASTQs were aligned in BowTie2 default mode. The output BAM file can be used for IGV visualization and mutations in the patient can be observed.

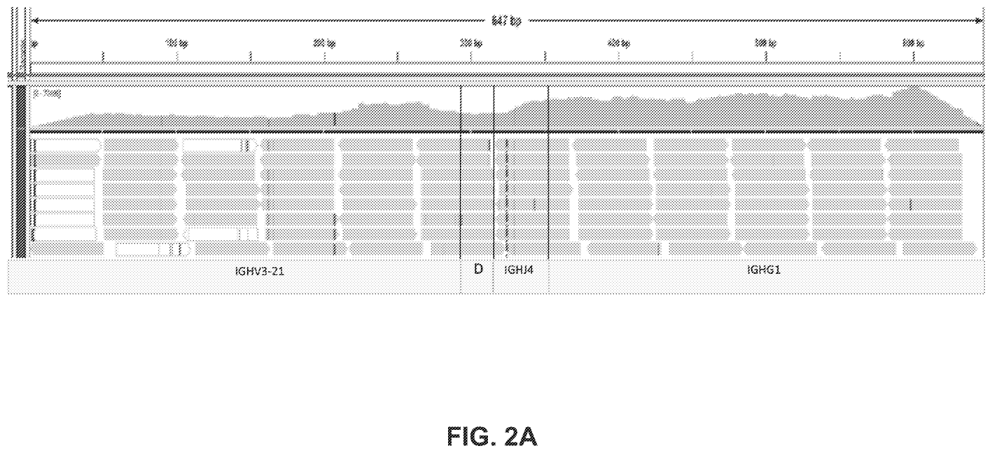

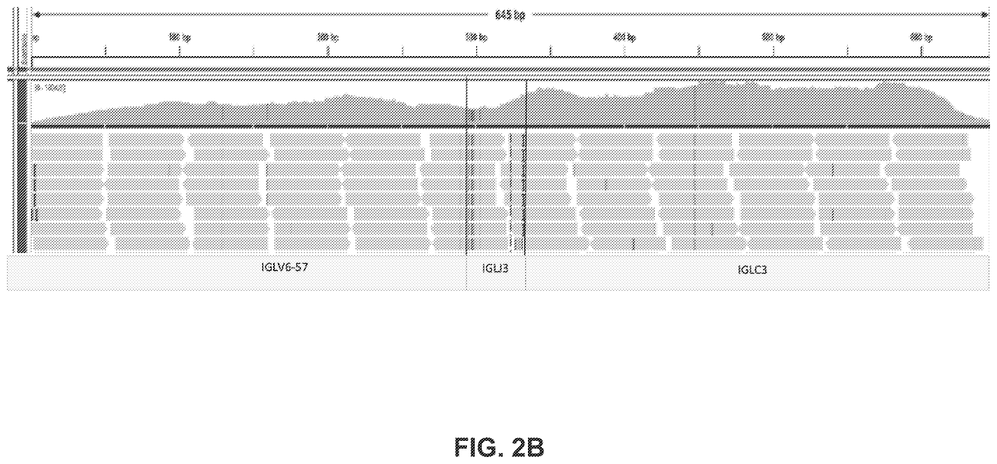

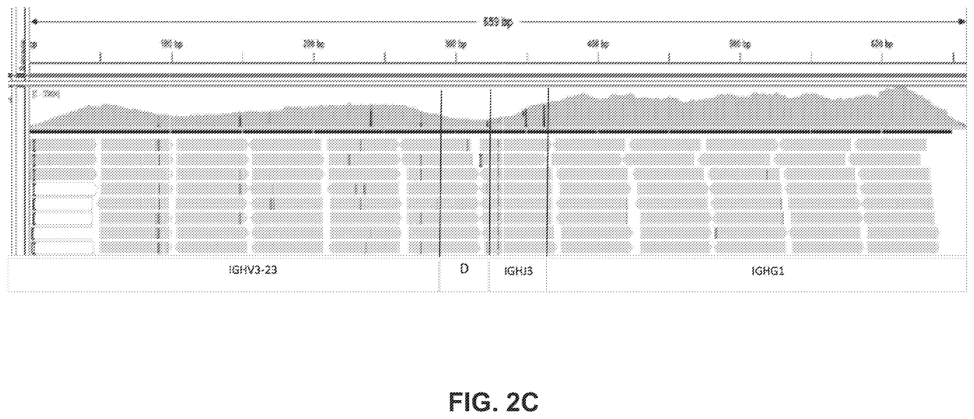

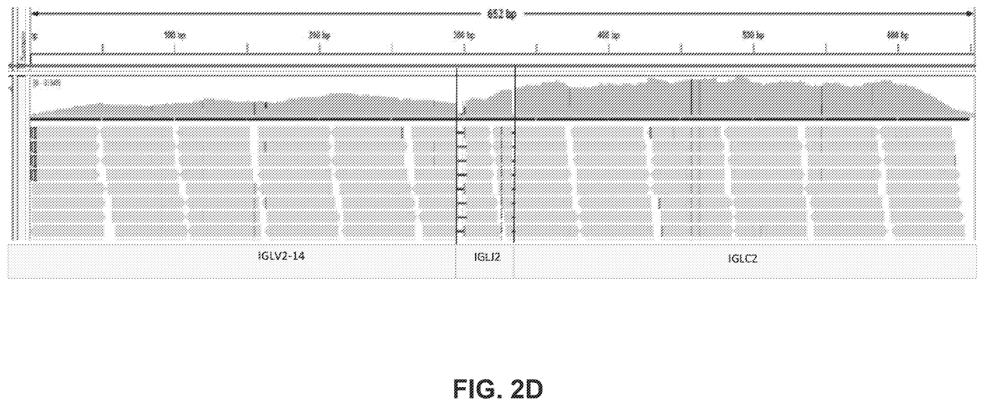

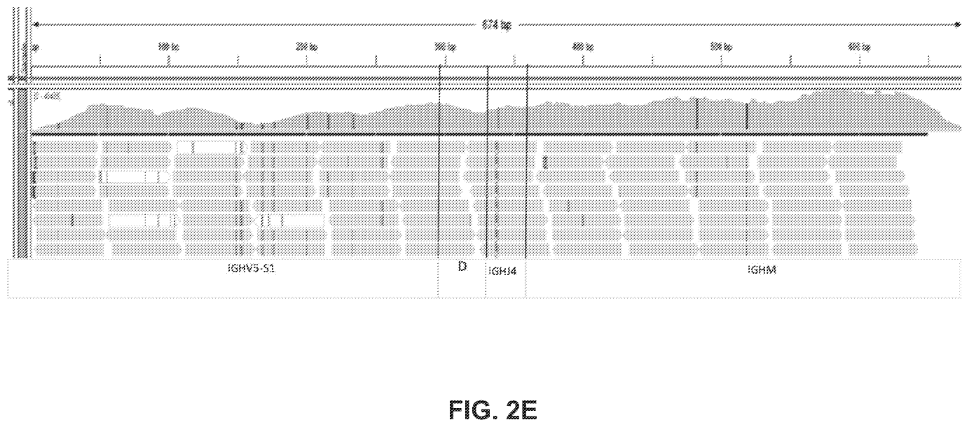

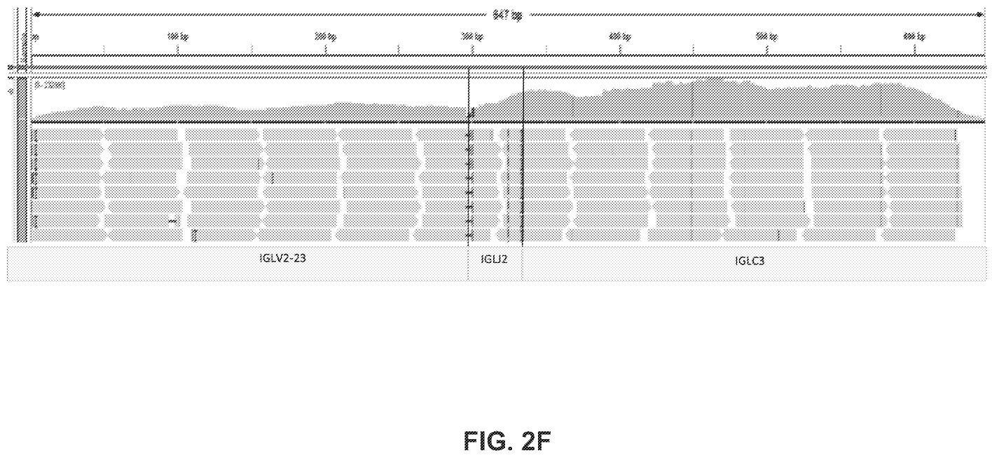

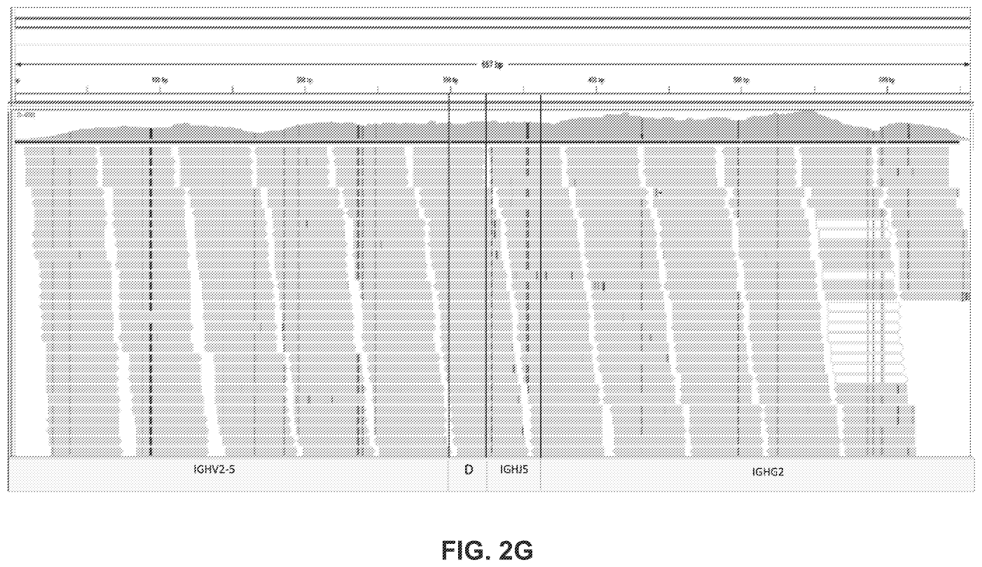

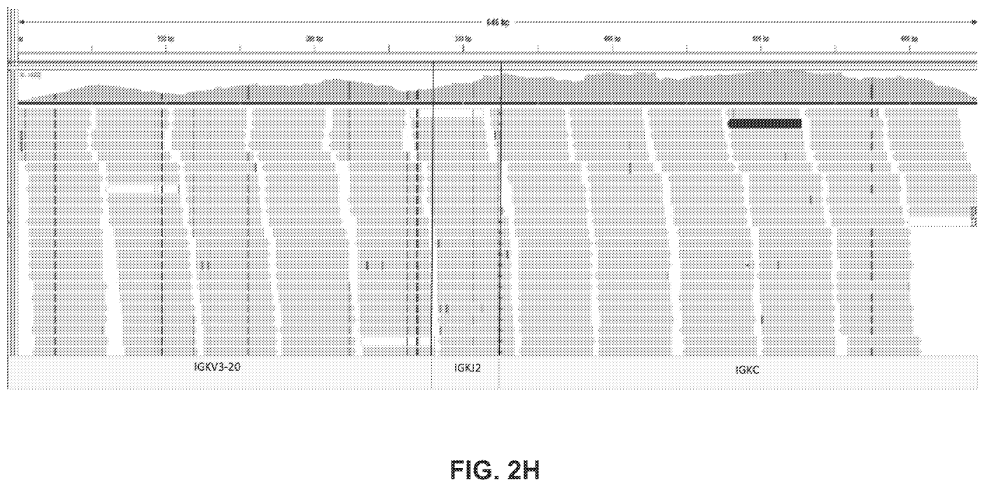

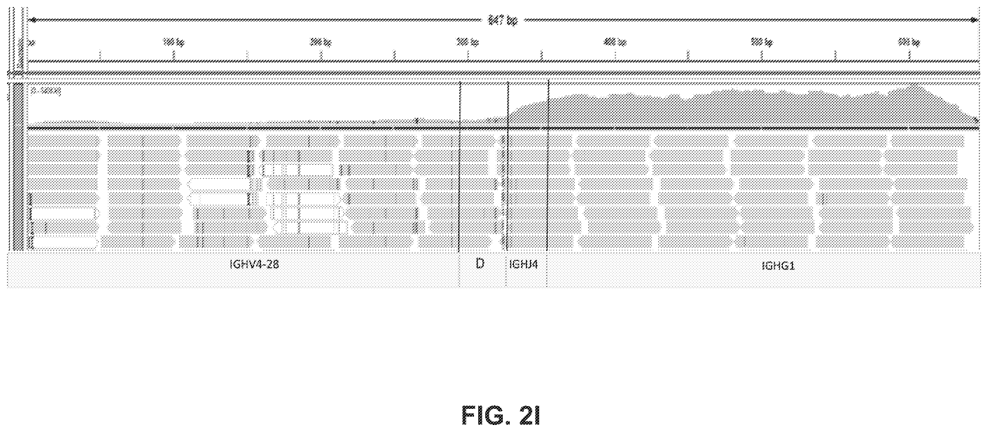

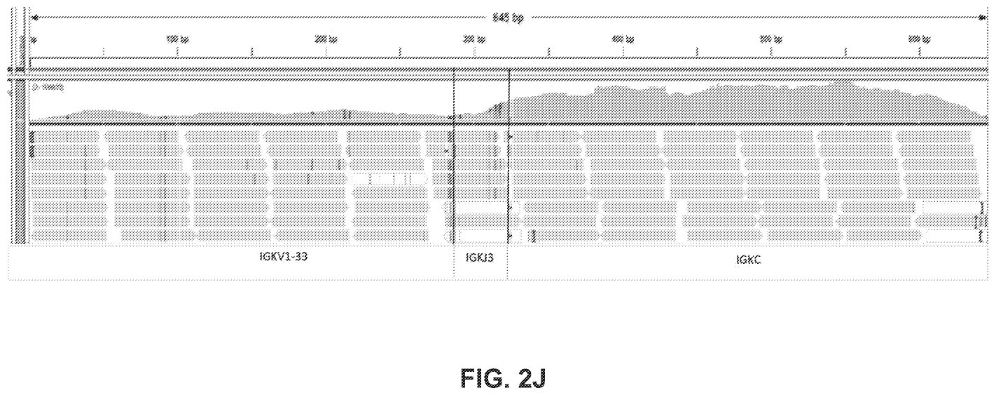

Example alignments and corresponding hypermutations using BowTie2 with default parameters for 4 exemplary patients are shown in FIG. 2A-2J. The D segments of the heavy chain was identified using a custom local assembly tool and edited the corresponding part of the FASTA file, therefore, no mutations are shown in D segments of IGV plots.

### Example 4: Identification of Rearranged Immunoglobulin CDR3 Amino Acid Sequences

The identification of the CDR3 region and corresponding V, D, and J chains from the final assembled FASTA sequences was achieved with IgBLAST. The standardized output using version v.1.9.0 of IgBLAST was delivered by wrapping IgBLASTn with default parameters. The output from the IgBLAST service is extracted using a purpose-built parser tool designed to extract the CDR1, CDR2 and CDR3 nucleotide and amino acid sequences. Summary of identified nucleotide and amino acid consensus sequences for CDR3 for the selected tumor samples are provided herein (e.g., Table 2 and Table 4).

### Example 5: VDJ Sequence Identification Workflow

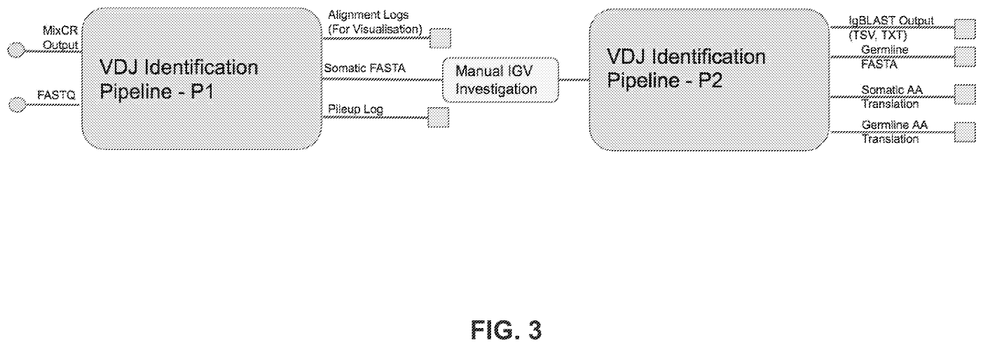

VDJ Sequence identification workflow was used to determine somatic and germline sequences of given patient and information such as CDR regions and mutation rates. The exemplary pipeline comprised of 3 steps (FIG. 3):
1. Somatic Sequence identification
2. Manual IGV investigation and (if necessary) correction of somatic vdj sequence
3. Germline Sequence and CDR regions identification

The workflow accepted 2 inputs for each target patient: (1) the TCGA Archive File: TCGA archive file of the patient. Prefixes of all output files were determined based on metadata (e.g. aliquot id) of patients' archive file; and (2) the preliminary alignment Output File: IG clones output of preliminary alignment were used to obtain initial segment id predictions. This text file included both heavy and light chain results.

By completing all three steps of the pipeline, the following output files were obtained:
Somatic Sequence: A FASTA file for a given patient's identified VDJ sequence
Germline Sequence: A FASTA file for a given patient's predicted germline sequence using the IMGT database.
The amino acid translation of Somatic and Germline FASTA files
IgBLAST output log for somatic FASTA file: Contains CDR regions
Alignment Logs: Visual text representation of the heavy D region and light V-J junction of somatic sequence (For validation purpose).
Pileup logs: Contains somatic mutation rate of segments and V-C segment coverage ratio of heavy and light chain which we use as an internal quality control metric.

### Step 1: Somatic Sequence Identification

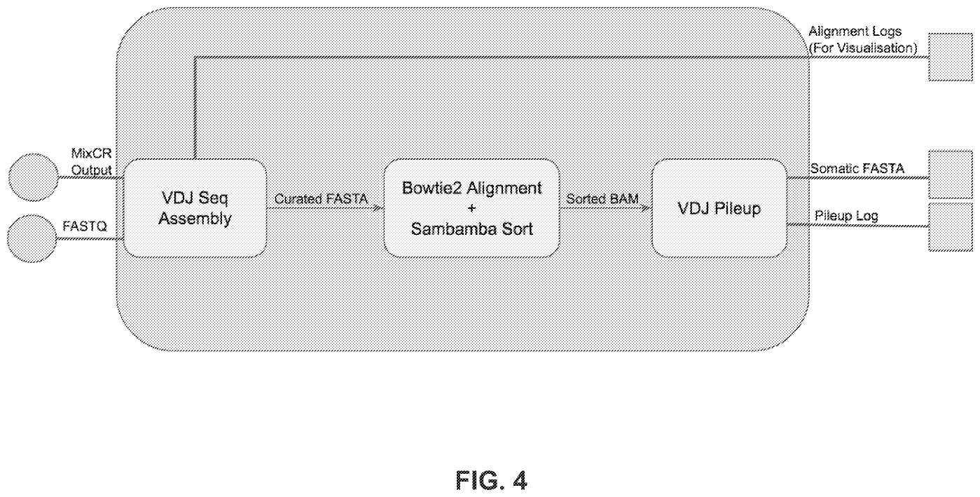

The first step of the VDJ sequence identification workflow was the somatic sequence identification. For this purpose, two input were initially taken, which were the IG segments id identified during the first alignment step and the FASTQ file of the patient. Somatic sequence identification was performed in 3 substages (FIG. 4):

### The Assembly Stage

During the preliminary alignment step, the vdjc segment ids were identified for both heavy and light chain. Then with use of the segment ids and IMGT database, the heavy and light chain sequences were generated by appending segment sequences to form V(D)JC structure.

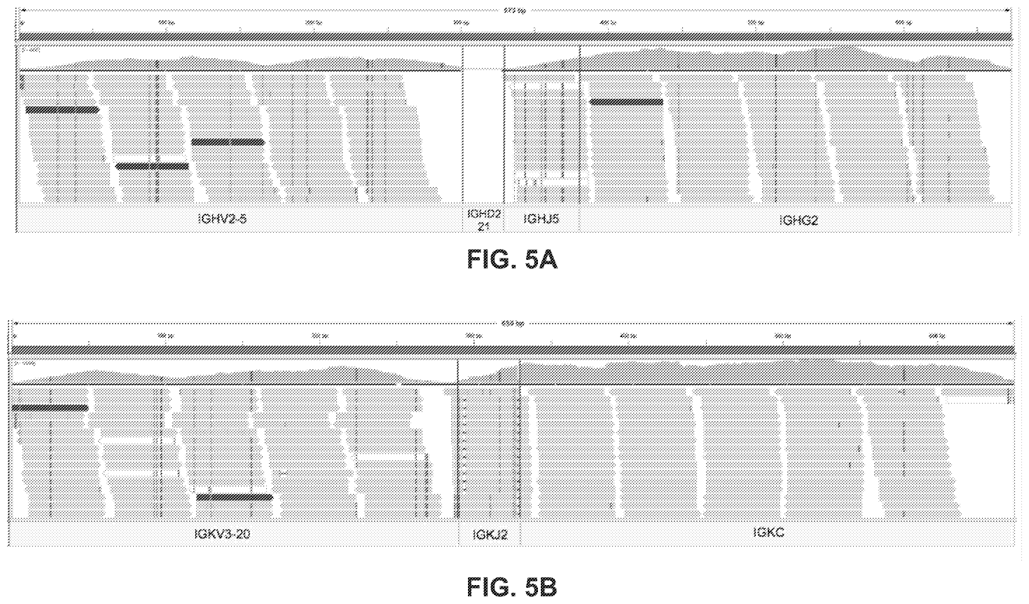

When the FASTQ of a patient was aligned with the reference FASTA generated by the first alignment step, it was often observed that D segment of the heavy chain (FIG. 5A) and V-J junction of the light chain (FIG. 5B) did not properly align. One reason of observing a low coverage in these areas could be the high mutation rate of antibody construction. Somatic mutations in these two regions are high enough that during the alignment against IMGT reference, many reads were eliminated. In addition, sizes of the reads were typically small for TCGA patients (e.g., 50 bp for Melanoma dataset) which was harder to align to difficult (mutated) regions.

In order to identify the correct sequence in heavy D and light V-J junction, a custom assembly based algorithm was implemented. From the VDJ segments identified during the first alignment step, a 22 bp seed sequence was selected from the ending of V segments. From the end of V segment, the read length was read backwards. From that index, the next 22 bp was selected as the initial seed. In some embodiments, the seed sequence is at least 10, 15, 20, 25, 30, 35, 40, 45, or 50 bp. In some embodiments, the seed sequence is no more than 10, 15, 20, 25, 30, 35, 40, 45, or 50 bp.

Once the seed sequence was selected, the FASTQ file was searched for the reads that contain this seed sequence. Since somatic mutations could occur, a fuzzy pattern searching algorithm was used (e.g. bitap algorithm) by allowing matches up to 4 edit distance penalty.

After the reads were selected in the first iteration, the unrelated ones were eliminated by comparing the whole read with V segment. The match ratio was checked of the intersection of reads and the V segment identified during the first alignment step. If the match ratio is less than 0.84, then the read was removed. Once the unrelated reads were removed, the reads were sorted descending by their match ratios and selected the first half of reads for pile up processing.

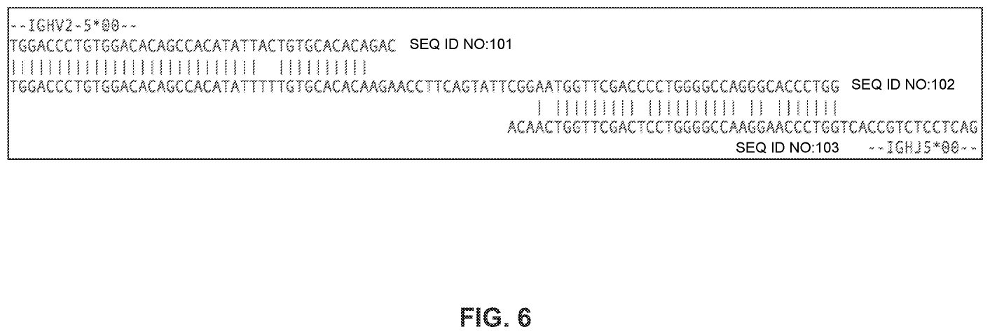

Using the selected reads, the bases were piled up and formed a single sequence. From the generated sequence, another 22 bp seed was selected and started a new iteration. For the following iterations, the maximum edit distance penalty was decreased to 1 and a read elimination was not performed in contrast to the first iteration. The iteration continued until a long enough final assembled sequence that covers more than half of the J segment was obtained (FIG. 6).

Once the assembled Heavy D region and Light V-J junction were obtained, the corresponding part of the reference was edited and produced an intermediate FASTA file for the alignment stage.

b. Alignment Stage

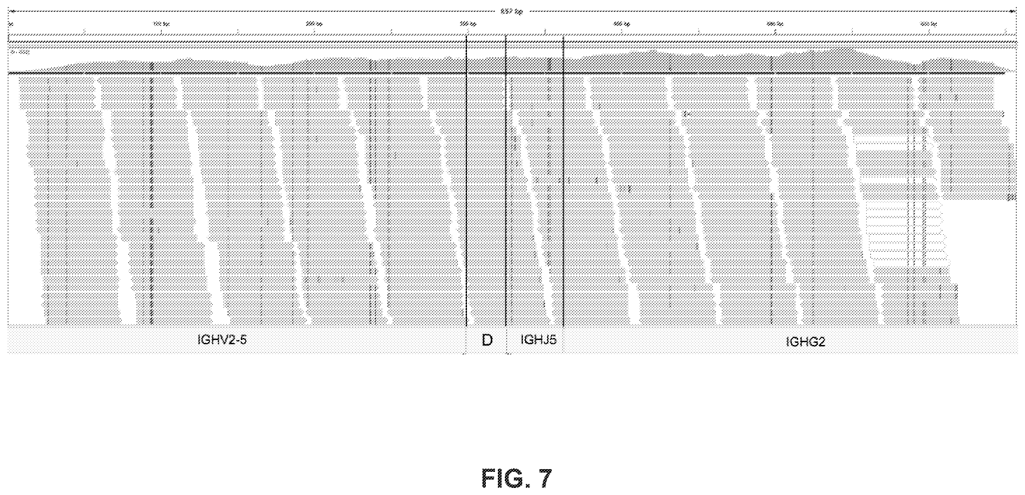

After the difficult regions (e.g. heavy D and light V-J junction) were identified using a custom assembly method, the aim was to correct the remaining variants (e.g. variants seen in FIG. 7) by using a standard variant calling pipeline which involved aligning reads followed by variant calling operation. For this purpose, BowTie2 2.2.6 with default parameters was used. To decrease the size of the output BAM file, the unaligned reads were discarded from the BAM file. After that, Sambamba 0.5.9 was used to sort the output BAM file.

c. Pileup Stage

In the third stage, rather than using a variant caller, the BAM file was used from the alignment stage do a pile-up processing to identify and correct variants in the reference file. For each position in the alignment, SNPs and INDELs were checked. Reads less than 20 quality threshold were ignored. In order to identify a variant in a specific position, 0.5 as the minimum ratio was applied, which meant that at least half of the total reads should contained that variant for the position. The variants in positions were also ignored where the total coverage is less than 200 reads. It was mostly observed that low coverage value in the first few base pairs of V segments and at the ending few base pairs of C segment.

### Mutation Rate Calculation

Once a final sequence was obtained, the sequence was compared with the initial reference file which the BAM file was generated from. The mutation rate was calculated as the Levenshtein Distance between segments divided by the Alignment Length of segments (e.g. Python Levenshtein.ratio (seq1, seq2)).

### Coverage Ratio Between V and C Segments

The average coverage was checked between V and C segments of both chains as an internal quality control step to ensure that the patient was high clonal. In the pileup log file, if the coverage ratio was over 0.3 then this suggested high clonality. A high V/C ratio might not always mean that the patient is highly clonal. However, a low V/C ratio could be a strong sign for low clonality.

### Step 2: Manual IGV Inspection & Somatic Sequence Correction

Once the somatic FASTA files were obtained through step 1, the FASTA file was manually inspected using IGV browser. The IGV browser was check on whether it showed a variant in our somatic reference file. Bases were mostly corrected which were previously skipped due to the low number of reads in pileup stage of step 1.

### Step 3: Germline Sequence and CDR Regions Identification

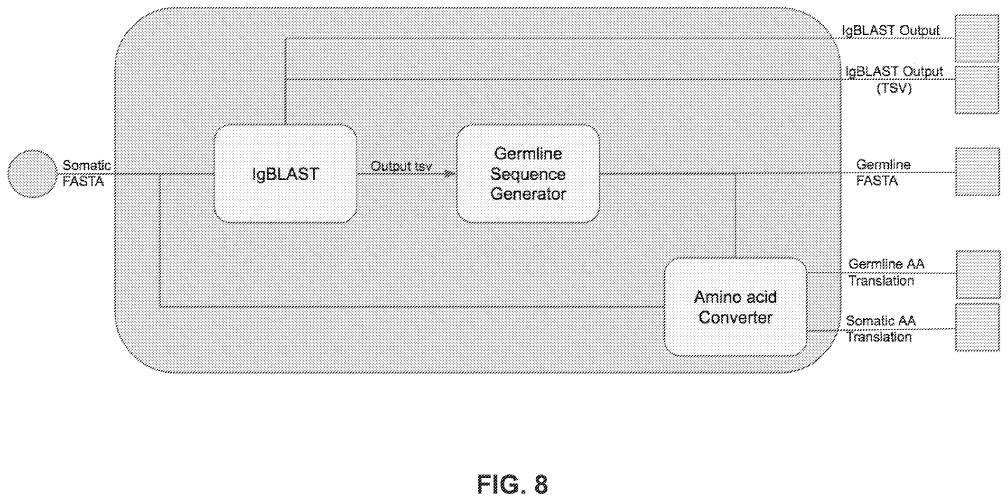

FIG. 8 illustrates a detailed schema of Germline and CDR sequence identification. Once a final somatic sequence was identified in the first two steps, the reference was inputted to the IgBLAST tool to identify the closest segment ids from the IMGT database. Once the closest ids was identified, a germline sequence was generated by merging the sequences from IMGT database in V(D)JC form.

IgBLAST also reported the positions of the CDR1, CDR2 and CDR3 sequences of the exemplary antibodies. Using those positions, the somatic sequence was clipped and the CDR regions returned with their amino acid translations.

As a final step, the amino acid translation of reconstructed complete germline and somatic VDJ consensus sequences was produced.

### Example 6: Identification of Dominant Ig Sequences in the TCGA Dataset

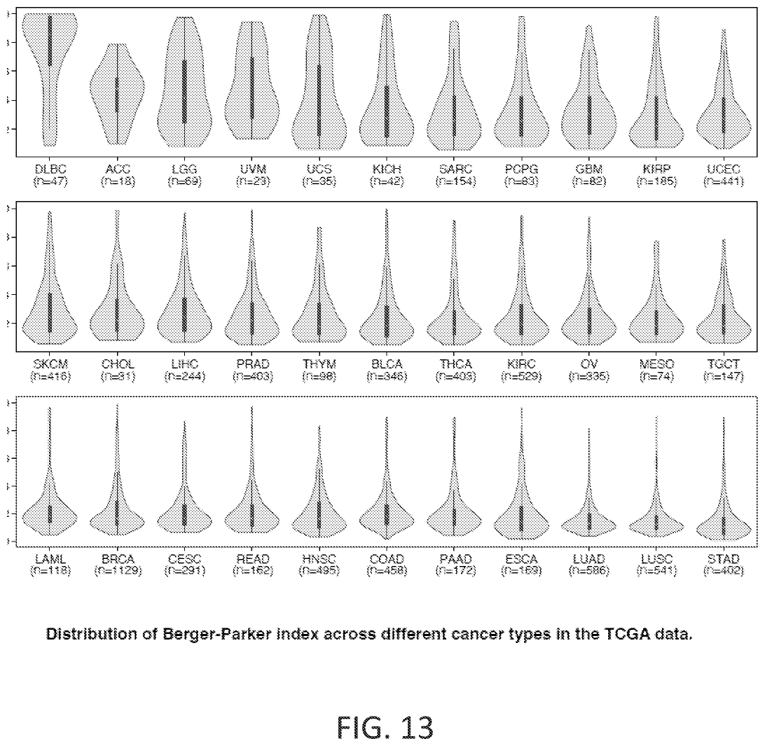

It has been observed that in some tumor samples the Ig repertoire is particularly clonal: a small set of Ig transcripts are expressed at high levels and account for the vast majority of all the reads attributable to Ig genes in that sample. We speculated that this phenomenon, which probably results from selected B cell clones winning the competition for the limited access to T follicular helper (Tfh) cells in the germinal center (GC) reaction, could be used to correctly pair heavy and light Ig chains from bulk RNA-Seq data, at least in some cases. Accordingly, we computationally assembled Ig chain sequences expressed in each TCGA tumor sample and we calculated the associated Berger-Parker index in order to identify samples with particularly dominant sequences. FIG. 13 depicts the distribution of Berger-Parker dominance scores of the heavy and light chains reconstructed from tumor samples in the TCGA dataset. As shown in FIG. 13, we observed that a small, but not insignificant, proportion of the analyzed samples expressed highly dominant Ig sequences.

The amount of sequencing reads not originating from immunoglobulin transcripts may be reduced through filtering. In this example, three filtering steps may be used. First, we select only the reads that either map to the immunoglobulin genes, or fail to map using (e.g.) Kallisto (version 0.44) (Bray et al. 2016) and Gencode (version 22) (Harrow et al. 2012) protein coding sequences as the reference. Second, we map the remaining reads to the full human genome (version GRCh38, (Harrow et al. 2012; Schneider et al. 2017)), again only keeping the reads that map to the immunoglobulin loci, or fail to map at all. Reads may then be extracted from the mapping and reverted to FASTQ format using Sambamba (Tarasov et al. 2015) view (version (0.5.9) and Samtools (Li et al. 2009) Fastq (version 1.8) commands. In a third filtering step, reads originating from viral and bacterial sources may be filtered by running kraken2 (Wood et al. 2019) and retaining unclassified reads.

Once reads have been filtered, they may be assembled using, e.g. Trinity RNA Seq assembler (version 2.8.4) (Grabherr et al. 2011) with custom parameters “-no_normalize_reads”, “-max_chrysalis_cluster_size 100”, and “-max_reads_per_graph 5000000”. We then map the assembled sequences to their germline V, D, and J regions using IgBLAST (Wood et al. 2019; Ye et al. 2013) (version 1.13). Sequences with high V gene match scores (cutoff=100) are kept as putative immunoglobulin chains.

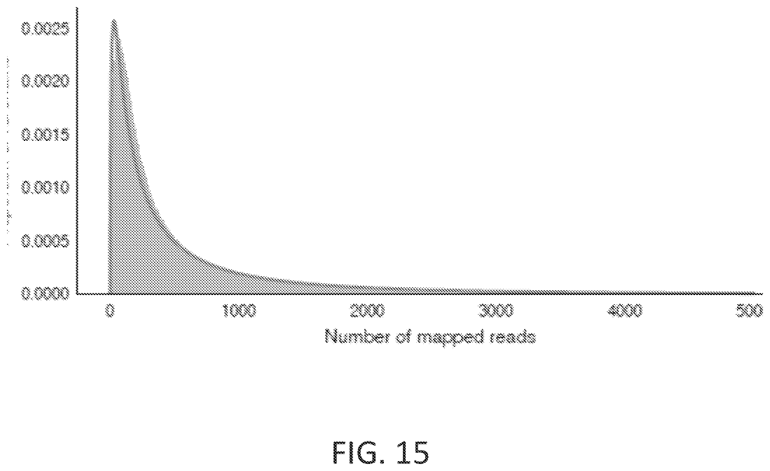

To evaluate the performance of this workflow and evaluate the possibility of identifying which heavy and light chain form a pair in cases where there is a strongly expressed clone, we generated a synthetic benchmark using single cell sequencing data for sample PW2 from Lindeman, I. et al., BraCeR: B-cell-receptor reconstruction and clonality inference from single-cell RNA-seq, Nat. Methods 15, 563-565 (2018). We estimated the distribution of Ig reads in TCGA data mapped to each reconstructed Ig chain in the and found it to closely follow a log-normal model, as illustrated in FIG. 15 (estimated mean=5.78, standard deviation—1.52). We then generated a set of 1,000 synthetic samples. We generated each sample by sampling 25 numbers from this distribution and assigning them as the total number of Ig reads to 25 random B cells from the PW2 data. We then randomly selected the corresponding number of RNA sequencing reads from those mapped to Ig chains in each of the 25 B cells. To this mix we also added 10 million reads randomly selected from a bulk RNA sequencing sample (TCGA-04-1348-01A), to act as background. We repeated this procedure 200 times for each value of the top clone reads, resulting in 1000 synthetic samples used in the benchmark. In evaluation, we considered the reconstruction to be a success if all of the CDR regions (CDR1, CDR2, and CDR3) for both the heavy and light chain of the top clone were correct, and the pair was correctly selected as the most abundant antibody.

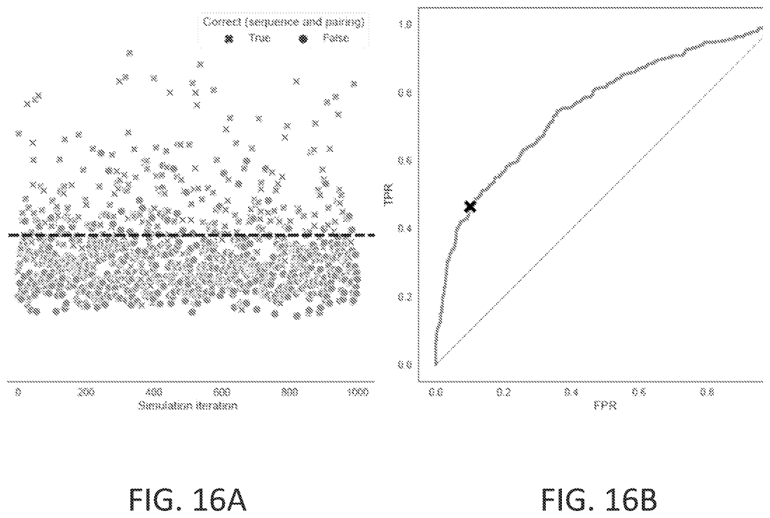

FIGS. 16A-B illustrate an evaluation of reconstruction performance on synthetic data. FIG. 16A shows the distribution of correctly and incorrectly reconstructed samples depending on the dominance score (e.g., the geometric mean of the Berger-Parker indices for heavy and light Ig chains) estimated by the workflow. Above the threshold of 0.382, the top antibodies for 90% of the synthetic samples were correctly reconstructed. FIG. 16B depicts a ROC curve for the evaluation at different values of the dominance score. The red cross marks the clonal cutoff of 0.382 selected as the point with the highest true positive rate (0.46) where the false positive rate is 0.1.

### Example 7: Dominant Ig Sequences can be Correctly Paired from Bulk RNA-Seq Data In-Silico

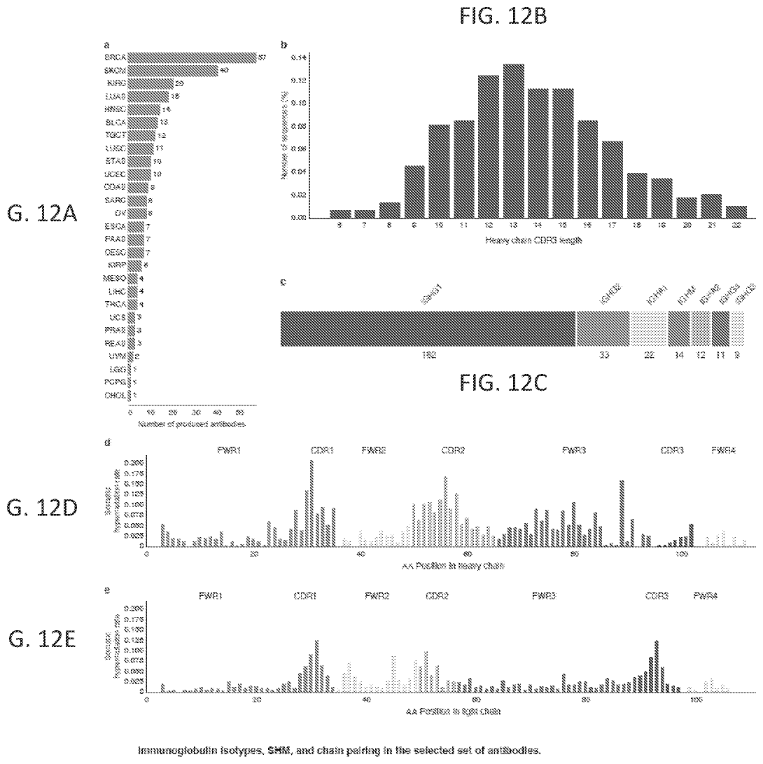

As a next step, we assessed whether the dominance information captured by the Berger-Parker index could be used to correctly pair heavy and light chains from bulk RNA-Seq data. In order to do so, we used RNA-Seq data from single B cells to simulate bulk RNA-Seq samples for which the expression level, sequence, and correct pairing of all Ig chains was known. Using this approach, we established that for samples with dominance scores larger than 0.382 we could identify and correctly pair the dominant Ig chains with high accuracy (true positive rate 0.46, false positive rate 0.1, AUC=0.757, as shown in FIGS. 16A-B). We used this result to select 1919 paired intratumoral Ig sequences from our TCGA analysis for expression in mammalian cells and further characterization, as shown in FIGS. 12A-F). As a control we also selected for expression and characterization 92 sequences below our chosen dominance score cutoff.

For each processed sample we used Kallisto to quantify the amount of RNA sequencing reads originating from each of the putative Ig chain transcripts. We then calculated the Berger-Parker dominance index for both the heavy and light Ig chains, as the proportion of reads attributed to the most common chain of the corresponding type. We then calculated a dominance score for each sample as the geometric mean of the heavy and light chain Berger-Parker indices. To evaluate the performance of this workflow, we generated a synthetic benchmark using single cell sequencing data (Lindman et al. 2018). Using the results of the synthethic benchmark, we determined the threshold on the score (>0.382) above which we expect the sequence reconstruction to be accurate, and the top (most abundant) heavy and light chain to form a paired Ig. For each such Ig we assigned the putative isotypes by mapping the constant portions of the heavy chains (sections of the assembled sequences post the annotated J regions) to the Gencode v22 reference immunoglobulin C sequences. We used the IgBlast mappings to the germline segment to identify the mutated positions in each chain. For every position as defined by the Martin numbering scheme (Abhinandan, K. R. & Martin, A. C. R., Analysis and Improvements to Kabat and structurally correct numbering of antibody variable domains, Mol. Immunol. 45, 3832-3839 (2008)) we calculated the mutation frequency across all of the reconstructed immunoglobulin chains.

### Example 8: Processing of TCGA RNA Sequencing Data

The raw RNA sequencing reads used are generated by the TCGA Research Network: https://www.cancer.gov/tcga, and are available through the TCGA data portal. TCGA BAM files were processed in the Cancer Genomics Cloud (Lau et al., The Cancer Genomics Cloud: Collaborative, Reproducible, and Democratized—A New Paradigm in Large-Scale Computational Research, Cancer Res. 77, e3-e6 (2017) (CGC). We applied sambamba-sort to sort the BAM files and Samtools fastq to convert them to FASTQ files, followed by Salmon quant in mapping-based mode (GENOME v27 (GRCH38.p10) transcript assembly was used) with GC bias correction. We obtained TPM values for 10533 tumor samples and 730 normal samples across 33 TCGA cancer types.

### Example 9: Antibody Sequences Reconstructed from Tumor RNA

In order to identify a putative subset of samples with clonally expanded B cell populations we applied a workflow according to an embodiment of the disclosure to TCGA RNA sequencing samples available in the Cancer Genomics Cloud (CGC) platform (n=11092). Consequently, we obtained antibodies that satisfy high confidence criteria for accurate sequence reconstruction and chain pairing for 28% of these samples (n=3074). For further experimental analysis we proceeded with a subset of 135 antibodies, derived from samples spread across different cancer types (FIG. 1a). These included both cancers traditionally considered immunologically hot (Posch et al. 2018; Bonaventura et al. 2019), such as melanoma (SKCM), bladder (BLCA), and lung cancer (LUAD), as well as those typically taken to be immunologically cold (Maleki Vareki 2018) like breast (BRCA), column (COAD), and pancreatic (PAAD) cancer. Lengths of the heavy chain complementarity-determining region three (CDR3) ranged between 7 and 22 amino acids, with the underlying distribution closely matching the expectation for a repertoire of human antibodies (Shi et al. 2014)(Shi et al. 2014; Hu et al. 2019). The most commonly used heavy chain V segment was IGHV3 (n=64, or 47.4%), followed by IGHV1 (n=30, or 22.2%), and IGHV4 (n=25 or 18.5%). Most light chains used IGKV1 (n=38, or 28.1%), followed by IGKV3 (n=26, or 19.3%) and IGLV3 (n=23, or 17%). Pairing frequencies largely agree with would be expected from random pairing, with IGHV3-IGKV1 being the most common pair (m=n=18, or 13.3% of all antibodies). The only notable exception was the IGHV4-IGKV1 pair, which occurred at double the expected frequency (10.4% vs 5.2%). However, in the larger set of 3074 putative antibodies, this pair occurs close to the expected frequency (4.6% vs 4.2%), suggesting that this representation is a consequence of our selection process, rather than a biological phenomena.

We next analysed these chains for evidence of somatic hyper-mutation (SHM) by mapping the RNA-Seq reads to the assembled sequences, examining the pileups for mismatching bases, and projecting the per nucleotide mutation frequencies onto amino acid positions as defined by the Martin numbering scheme(Abhinandan and Martin 2008). We found the mutational profiles of both the heavy and the light chains to be largely in agreement with previous reported findings (Yaari et al. 2013) (Yaari et al. 2013; Saul et al. 2016), with most SHM concentrated in the complementarity-determining regions one and two (CDR1 and CDR2).

FIGS. 12A-F depict various properties of antibodies identified according to embodiments of the disclosure. FIG. 12A shows the distribution of antibodies across TCGA cancer types. FIG. 12B shows the distribution of amino acid lengths of IgH CDR3 regions. FIG. 12C shows the number of selected antibodies by isotype. FIGS. 12D-E show the mean SHM rate for each position in the Martin numbering scheme across the heavy and light chains in the selected set of antibodies. For each chain, per amino-acid mutation rates were estimated from the mapped sequencing reads and numbered following the Martin numbering scheme. For chains where multiple amino acids map to the same Martin number, we used the mean value across those amino acids.

### Example 10: In-Silico Paired Ig Sequences can be Expressed at High Levels in Mammalian Cells

We performed gene synthesis for 283 paired Ig sequences and attempted expression of the corresponding antibody proteins in mammalian cells (HEK293). For each candidate, we replaced the heavy constant region with a standard human IgG1 sequence in order to facilitate subsequent detection and screening. The variable region of the antibodies were recombined with a constant region of human IgG class I using AbAb's recombinant platform (Absolute Antibody Ltd, Oxford, UK). The antibodies were expressed into HEK293 cells using the Absolute Antibody transient expression system and purified by one-step affinity chromatography. We performed Quality Control (QC) analysis to assess concentration, total amount, level of endotoxin, and sodium dodecyl sulfate polyacrylamide gel electrophoresis (SDS-PAGE). We successfully expressed 275 recombinant antibodies out of 283 with high yield (mean yield 2.35 mg/150 mL). After expression, each antibody was purified using protein A, stored in PBS at a standard concentration of 1 ug/uL and used for subsequent target antigen screening.

### Example 11: Identification of Target Antigens Using High-Throughput Proteomics

Having successfully expressed the intratumoral Ig we paired in-silico, we decided to use the resulting recombinant antibodies to attempt identification of their target antigens. To this aim, we used high throughput proteomics to screen individual antibodies against a large collection of wild-type human proteins. We screened 173 antibodies against a collection of about twenty thousand human proteins, each represented in duplicate on the surface of a protein array, covering about 80% of the known human proteome. Specifically, we tested the antibodies using the human proteome microarray HuProt™ 4.0, a comprehensive library of GST-tagged recombinant human protein expressed in yeast. Briefly, the HuProt arrays were blocked with 5% BSA/1×TBS-T at room temperature for 1 hour. Then antibodies were incubated at 4° C. overnight, washed, and labelled with secondary antibodies prior to detection.

While protein arrays are a cost-effective way to test antibodies against a large number of potential targets, this technology is not designed to display membrane proteins in their correct conformation. Complex membrane proteins are unlikely to fold correctly when immobilized on the surface of the array, outside or their native membrane context. For this reason, we used high-throughput fluorescence-activated cell sorting (HT-FACS) to screen 92 antibodies against a library of six thousand structurally intact membrane proteins expressed in human HEK0293T cells. Specifically, we used the Membrane Protein Array (MPA), which is Integral Molecular's cell-based array of ˜6,000 human membrane proteins, each expressed in live unfixed cells in separate wells of a 384-well plate. In this study, the MPA was expressed in HEK-293T cells 36 h prior to testing. Each MAb was fluorescently labeled and added to the MPA at a concentration optimized for the best signal-to-background ratio for target detection using an independent immunofluorescence titration curve against membrane-tethered protein A. Binding was measured by Intellicyt iQue3. Each 384-well plate contained positive (Fc-binding) and negative (empty vector) controls to ensure plate-by-plate data validity. Hits were validated by flow cytometry with serial dilutions of antibody, and the target identity was confirmed by sequencing.

Statistical analysis of antigen arrays. Raw signal intensities (mean F635 values) were correlated for background signal (mean B635 values) using the function background from R package limma, with the correction method set to ‘normexpr’ with ‘mle’ parameter estimation strategy. The replicate spots for the same target clone were summarized by calculating the geometric mean followed by logarithmic transformation. We noticed that a number of spots reported high signal intensities across multiple arrays likely due to non-specific binding. To counter this issue we subtracted the mean signal value across all of the 172 arrays for each target clone. We then centered and scaled the signal intensities and performed multiple testing corrections by calculating the false discovery rate according to the Benjamini-Hochberg method. We used a stringent q-value cutoff of 0.01 in order to control the number of false positive hits.

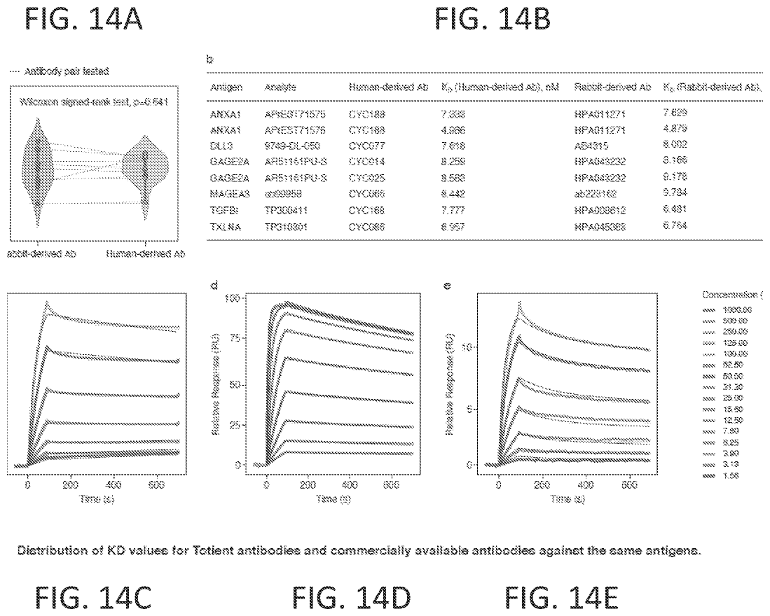

As a result of these screenings, we were able to identify high confidence hits for 84 of our antibody candidates screened using protein arrays (48%) and 21 of candidates screened using HT-FACS (23%). Targets recognized by our in-silico paired intratumoral Ig included well known cancer-specific antigens1 4 (NY-ESO-1, MAGEA3, GAGE2A, DLL3) as well as immunomodulatory molecules expressed in the tumor microenvironment (ANXA1, TGFBI, C4BPB; see FIGS. 14A-E).

### Example 12: Intratumoral Ig Bind their Target Antigens with High-Affinity

As a next step, we decided to independently confirm the interaction between selected intratumoral Ig and their putative target antigens by using surface plasmon resonance (SPR). For each intratumoral Ig sequence, we characterized the binding affinity of the corresponding recombinant antibody to its putative target antigen, having sourced the antigen independently of the vendor used for the high-throughput proteomics screening. A Biacore 8K instrument, exploiting the SPR principle, was used to calculate the equilibrium dissociation constant (K_{D}). Selected antibodies (ligand) were individually immobilized to a sensor chip coated with Protein A to ensure the correct orientation of the antibody while the antigens (analyte) were injected within the flow stream at 30 l/min. We used 90s association time for each experiment with 600s dissociation time. Data analysis was performed using the R package pbm. Sensorgram raw data time series measurements were downloaded from the Biacore 8K instrument and fitted to an appropriate observation model from the pbm package using non-linear least squares parameter estimation techniques. Models were selected parsimoniously; those which could adequately explain the data using fewer parameters were generally preferred. In a small number of cases, selected concentration curves were excluded from the fitting procedure due to either instrumental measurement anomalies, or apparent statistical outliers. Examples of qualifying anomalies included refractive index bulk shift discontinuities at the transition between the association and dissociation measurement phases.

We confirmed 19 antibody-antigen interactions. We observed that the recombinant antibodies derived from intratumoral Ig sequences bind to their target antigen with very high affinity, with K_{D} in the low nanomolar range (FIG. 14B). When we compared our fully human antibodies against commercial antibodies obtained from rabbits (a model organism known to generate very high affinity antibodies) after immunization with the same antigen, we found no significant differences in their binding affinity (FIG. 14A).

FIG. 14A depicts the distribution of empirically determined K_{D} values for human-derived antibodies (Abs) and their rabbit-derived counterparts for the same antigens, showing no statistically significant difference using a paired t-test. As Abs have been tested in multiple experiments, to calculate the p-value, we first averaged the −log 10(K_{D}) across the experiments with a specific analyte (different source of antigen), then paired each of these averages from human-derived Abs with averages from rabbit-derived Abs for the same analyte and applied paired t-test. FIG. 14B depicts K_{D} values for human and rabbit-derived antibodies presented in FIG. 14A. When multiple experiments have been performed for a human or rabbit Ab, we calculated the average K_{D} from these experiments. FIGS. 14C-E depicts representative sensorgrams of SPR-determined antibody-antigen interaction for CYC214 (anti-C4BPB antibody) (FIG. 14C), CYC066 (anti-MAGEA3 antibody) (FIG. 14D) and CYC168 (ant-TGFBI antibody) (FIG. 14E). Solid shaded lines indicate raw data observed by Biacore 8K instrument, and overlaid solid black lines indicate the fit result estimated using the model.

### Example 13: Epitope mapping for recombinant antibodies derived from intratumoral Ig

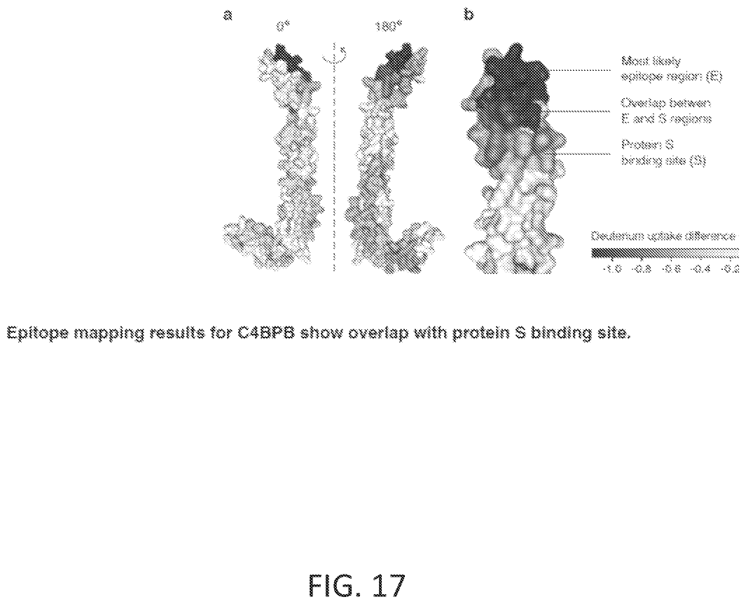

Having demonstrated that our in-silico paired intratumoral Ig bind to their target antigens with high-affinity, we decided to assess whether it would be possible to identify their epitope, at least in principle. We selected one corresponding recombinant antibody and performed epitope mapping using hydrogen/deuterium exchange mass spectrometry (HDX-MS). Linear peptides spanning the target antigen length were incubated in a deuterium containing buffer in presence or absence of the relevant antibody to observe differential exchange with hydrogen at the putative binding sites. Using this technique, we were able to identify the putative binding region of the antibody on its designated target (C4BPB; FIG. 17). We observed that the most likely epitope overlaps with the C4BPB binding site for protein S1 9, which is crucial for its biological function2 0. Although additional functional studies would be required to clarify whether or not this particular antibody is able to interfere with the binding between C4BPB and protein S, our workflow shows that in principle it is possible to identify the target antigen and the corresponding epitope for in-silico paired intratumoral Ig, thus gaining further insights into their biological function.

FIG. 17 is a graphical representation of the epitope mapping results, showing C4BPB overlap with the protein S binding site. HDX-MS was used to measure the level of deuterium (D) uptake by C4BPB alone or in presence of CYC214 antibody. FIG. 17 at left shows the relative D uptake difference per residue (shaded light to dark) across the entire protein surface. FIG. 17 at right shows details of the protein region containing the known binding site 20 for protein S. High D uptake difference (dark shading) is detected in the region containing the binding site for protein S, thus suggesting that CYC214 might be disrupting the interaction between C4BPB and protein S. Each C4BPB fragment is measured up to three times and each residue can be covered by one or more overlapping fragments: the uptake difference per residue was calculated using the mean of the uptake differences of the fragments covering the residue.

### Exemplary Results

Exemplary reconstructed amino acid and nucleic acid consensus sequences of variable heavy chain, variable light chain and their corresponding CDR3 are provided below.

Table 1 lists exemplary reconstructed amino acid consensus sequences of variable heavy chain (VH) and Exemplary reconstructed amino acid consensus sequences variable light chain (VL).

| | | SEQ ID | |
|---|---|---|---|
| Name | Chain | No | Amino acid sequences |
| TMEL1001 | VH | 1 | CEVQLVESGGGLVKPGGSLRLSCAASGFTFRSYSMNWVRQ |
| | | | APGKGLEWVSSISSSGNYIYYADSVKGRFTLSRDNAKNSLY |
| | | | LQMNSLRAEDTAVYYCARGGGTSWSHYWGQGTLVTVSS |
| | | | ASTKGPSVFPLAPSSKSTSGGTAALGCLVKDYFPEPVTVSW |
| | | | NSGALTSGVHTFPAVLQSSGLYSLSSVVTVPSSSLGTQTYIC |
| | | | NVNHKPSNTKVDKSE |
| | VL | 6 | QFMLTQPHSVSESPGKTVTISCTRSSGSIASNYVQWYQQRP |
| | | | GSAPTTVIYEDNERPSGVPDRFSGSIDSSSNSASLTISGLKTE |
| | | | DEADYYCQSYDSNNRWVFGGGTKLTVLGQPKAAPSVTLF |
| | | | PPSSEELQANKATLVCLISDFYPGAVTVAWKADSSPVKAG |
| | | | VETTTPSKQSNNKYAASSYLSLTPEQWKSHKSYSCQVTHE |
| | | | GSTVEKTVAPTECS |
| TMEL1002 | VH | 2 | VVQLLESGGGLVQPGGSLRLSCAASGFTFSSYAMSWVRQA |
| | | | PGKGLEWVSAISGSGGSTYYADSVKGRFTISRDNSKNTLYL |
| | | | QMNSLRAEDTAVYYCAKDAYDSSGPDAFDIWGQGTMVT |
| | | | VSSASTKGPSVFPLAPSSKSTSGGTAALGCLVKDYFPEPVT |
| | | | VSWNSGALTSGVHTFPAVLQSSGLYSLSSVVTVPSSSLGTQ |
| | | | TYICNVNHKPSNTKVDKRV |
| | VL | 7 | ASALTQPASVSGSPGQSITISCTGTSSDVGDYNYVSWYQQH |
| | | | PGKAPKLMIYDVSNRPSGVSNRFSGSKSGNTASLTISGLQA |
| | | | EDEADYYCSSYTSSSTLVFGGGTKLTVLGQPKAAPSVTLFP |
| | | | PSSEELQANKATLVCLISDFYPGAVTVAWKADSSPVKAGV |
| | | | ETTTPSKQSNNKYAASSYLSLTPEQWKSHRSYSCQVTHEGS |
| | | | TVEKTVAPTECS |
| TMEL1003 | VH | 3 | AVQLVQSGAEVKKPGESLRISCKGSGYSFTSYWISWVRQM |
| | | | PGKGLEWMGRIDPSDSYTNYSPSFQGHVTISADKSISTAYL |
| | | | QWSSLKTSDTAMYYCARPLQTYSIASVGHWGQGTLVTVSS |
| | | | GSASAPTLFPLVSCENSPSDTSSVAVGCLAQDFLPDSITFSW |
| | | | KYKNNSDISSTRGFPSVLRGGKYAATSQVLLPSKDVMQGT |
| | | | DEHVVCKVQHPNGNKEKNVPLP |
| | VL | 8 | ASALTQPASVSGSPGQSITISCTGTSSDVGSYNLVSWYQQH |
| | | | PGKAPKLMIYEGSKRPSGVSNRFSGSKSGNTASLTISGLQA |
| | | | EDEADYYCCSYAGSSTFAVFGGGTKLTVLGQPKAAPSVTL |
| | | | FPPSSEELQANKATLVCLISDFYPGAVTVAWKADSSPVKAG |
| | | | VETTTPSKQSNNKYAASSYLSLTPEQWKSHRSYSCQVTHE |
| | | | GSTVEKTVAPTECS |
| TMEL1004 | VH | 4 | QITLKESGPTLVKPTQTLTLTCTFSGFSLNTPGVGVGWIRQP |
| | | | PGKALEWLALIYWDDDKRYRPSLESRLTITKDTSKNHVVL |
| | | | TMTNMDPVDTATYFCAHKNLQYSEWFDPWGQGTLVIVSS |
| | | | ASTKGPSVFPLAPCSRSTSESTAALGCLVKDYFPEPVTVSW |
| | | | NSGALTSGVHTFPAVLQSSGLYSLSSVVTVPSSSLGTKTYT |
| | | | CNVDHKPSNTKVDKRV |
| | VL | 9 | EMVLTQSPATLSLSPGERATLSCRASQSVSRNSLAWYQQRP |
| | | | GQTPRLLIYGASSRATGIPDRESGSGSGTDFTLIISRLEPEDF |
| | | | AVYFCLQYDESPYTFGQGAKLEIKRTVAAPSVFIFPPSDEQL |
| | | | KSGTASVVCLLNNFYPREAKVQWKVDNALQSGNSQESVT |
| | | | EQDSKDSTYSLSSTLTLSKADYEKHKLYACEVTHQGLSSPV |
| | | | TKSFNRGEC |
| TMEL1005 | VH | 5 | PVQLQESGPGLVKPSETLSLTCTVSGGSMSIRSSYWGWIRQ |
| | | | SPGKGLEWIGHIFYSGSTYYNPSLQSRVTILVDTSKNQFSLR |
| | | | LSSVTAADTAVYYCVRSFGVARWDFWGQGTLVTVSSAST |
| | | | KGPSVFPLAPSSKSTSGGTAALGCLVKDYFPEPVTVSWNSG |
| | | | ALTSGVHTFPAVLQSSGLYSLSSVVTVPSSSLGTQTYICNVN |
| | | | HKPSNTKVDKKV |
| | VL | 10 | DIQMTQSPSSLSASVGDRVTITCQASQNIRNYLNWYQQKPG |
| | | | KAPKLLIYDASNLETGVPSRFSGSGSGTDFTLTISSLQPEDIA |
| | | | TYYCQQYDNLLLFTFGPGTTVDIKRTVAAPSVFIFPPSDEQL |
| | | | KSGTASVVCLLNNFYPREAKVQWKVDNALQSGNSQESVT |
| | | | EQDSKDSTYSLSSTLTLSKADYEKHKVYACEVTHQGLSSP |
| | | | VTKSFNRGEC |

Table 2 below lists exemplary reconstructed amino acid consensus sequences of complementarity-determining region 3 from a variable heavy chain (CDR-H3) and exemplary reconstructed amino acid consensus sequences of complementarity-determining region from a variable light chain (CDR-L3)

| | | | Amino acid sequences |
|---|---|---|---|
| | Region | SEQ | of complementarity- |
| | and | ID | determining |
| Name | Chain | No | region 3 (CDR3) |
| TMEL1001 | CDR-H3 | 11 | ARGGGTSWSHY |
| | CDR-L3 | 16 | QSYDSNNRWV |
| TMEL1002 | CDR-H3 | 12 | AKDAYDSSGPDAFDI |
| | CDR-L3 | 17 | SSYTSSSTLV |
| TMEL1003 | CDR-H3 | 13 | ARPLQTYSIASVGH |
| | CDR-L3 | 18 | CSYAGSSTFAV |
| TMEL1004 | CDR-H3 | 14 | AHKNLQYSEWFDP |
| | CDR-L3 | 19 | LQYDESPYT |
| TMEL1005 | CDR-H3 | 15 | VRSFGVARWDF |
| | CDR-L3 | 20 | QQYDNLLLFT |
| TMEL1001 | CDR-H2 | 21 | ISSSGNYI |
| | CDR-L2 | 26 | EDN |
| TMEL1002 | CDR-H2 | 22 | ISGSGGST |
| | CDR-L2 | 27 | DVS |
| TMEL1003 | CDR-H2 | 23 | IDPSDSYT |
| | CDR-L2 | 28 | EGS |
| TMEL1004 | CDR-H2 | 24 | IYWDDDK |
| | CDR-L2 | 29 | GAS |
| TMEL1005 | CDR-H2 | 25 | IFYSGST |
| | CDR-L2 | 30 | DAS |
| TMEL1001 | CDR-H1 | 31 | GFTFRSYS |
| | CDR-L1 | 36 | SGSIASNY |
| TMEL1002 | CDR-H1 | 32 | GFTFSSYA |
| | CDR-L1 | 37 | SSDVGDYNY |
| TMEL1003 | CDR-H1 | 33 | GYSFTSYW |
| | CDR-L1 | 38 | SSDVGSYNL |
| TMEL1004 | CDR-H1 | 34 | GFSLNTPGVG |
| | CDR-L1 | 39 | QSVSRNS |
| TMEL1005 | CDR-H1 | 35 | GGSMSIRSSY |
| | CDR-L1 | 40 | QNIRNY |

Table 3 below lists exemplary reconstructed nucleic acid consensus sequences of variable heavy chain (VH) and exemplary reconstructed nucleic acid consensus sequences of variable light chain (VL)

| | | SEQ ID | |
|---|---|---|---|
| Name | Chain | No | Nucleic acid sequences |
| TMEL1001 | VH | 41 | GTGTGAGGTGCAGCTGGTGGAGTCTGGGGGAGGCCTGGTCAAGCCTGGGG |
| | | | GGTCCCTGAGACTCTCCTGTGCAGCCTCTGGATTCACCTTCAGGAGCTATAG |
| | | | CATGAACTGGGTCCGCCAGGCTCCAGGGAAGGGGCTGGAGTGGGTCTCATC |
| | | | CATTAGTAGTAGTGGTAATTACATATACTACGCAGACTCAGTGAAGGGCCG |
| | | | ATTCACCCTCTCCAGAGACAACGCCAAGAACTCACTGTATCTGCAAATGAA |
| | | | CAGCCTGAGAGCCGAGGACACGGCTGTGTATTACTGTGCGAGAGGTGGGG |
| | | | GTACCAGCTGGTCGCATTACTGGGGCCAGGGAACCCTGGTCACCGTCTCCT |
| | | | CAGCCTCCACCAAGGGCCCATCGGTCTTCCCCCTGGCACCCTCCTCCAAGA |
| | | | GCACCTCTGGGGGCACAGCGGCCCTGGGCTGCCTGGTCAAGGACTACTTCC |
| | | | CCGAACCGGTGACGGTGTCGTGGAACTCAGGCGCCCTGACCAGCGGCGTGC |
| | | | ACACCTTCCCGGCTGTCCTACAGTCCTCAGGACTCTACTCCCTCAGCAGCGT |
| | | | GGTGACCGTGCCCTCCAGCAGCTTGGGCACCCAGACCTACATCTGCAACGT |
| | | | GAATCACAAGCCCAGCAACACCAAGGTGGACAAGAGTGAG |
| | VL | 46 | CAATTTATGCTGACTCAGCCCCACTCTGTGTCGGAGTCTCCGGGGAAGACG |
| | | | GTAACCATCTCCTGCACCCGCAGCAGTGGCAGCATTGCCAGCAACTATGTG |
| | | | CAGTGGTACCAGCAGCGCCCGGGCAGTGCCCCCACCACTGTGATCTATGAG |
| | | | GATAACGAAAGACCCTCTGGGGTCCCTGATCGGTTCTCTGGCTCCATCGAC |
| | | | AGCTCCTCCAACTCTGCCTCCCTCACCATCTCTGGACTGAAGACTGAGGACG |
| | | | AGGCTGACTACTACTGTCAGTCTTATGATAGCAACAATCGTTGGGTGTTCGG |
| | | | CGGAGGGACCAAGCTGACCGTCCTAGGTCAGCCCAAGGCTGCCCCCTCGGT |
| | | | CACTCTGTTCCCACCCTCCTCTGAGGAGCTTCAAGCCAACAAGGCCACACT |
| | | | GGTGTGTCTCATAAGTGACTTCTACCCGGGAGCCGTGACAGTGGCCTGGAA |
| | | | GGCAGATAGCAGCCCCGTCAAGGCGGGAGTGGAGACCACCACACCCTCCA |
| | | | AACAAAGCAACAACAAGTACGCGGCCAGCAGCTACCTGAGCCTGACGCCT |
| | | | GAGCAGTGGAAGTCCCACAAAAGCTACAGCTGCCAGGTCACGCATGAAGG |
| | | | GAGCACCGTGGAGAAGACAGTGGCCCCTACAGAATGTTCA |
| TMEL1002 | VH | 42 | GTGGTGCAGCTGTTGGAGTCTGGGGGAGGCTTGGTACAGCCTGGGGGGTCC |
| | | | CTGAGACTCTCCTGTGCAGCCTCTGGATTCACCTTTAGCAGCTATGCCATGA |
| | | | GCTGGGTCCGCCAGGCTCCAGGGAAGGGGCTGGAGTGGGTCTCAGCTATTA |
| | | | GTGGTAGTGGTGGTAGCACATACTACGCAGACTCCGTGAAGGGCCGGTTCA |
| | | | CCATCTCCAGAGACAATTCCAAGAACACGCTGTATCTGCAAATGAACAGCC |
| | | | TGAGAGCCGAGGACACGGCCGTATATTACTGTGCGAAAGATGCATATGATA |
| | | | GTAGTGGCCCAGATGCTTTTGATATCTGGGGCCAAGGGACAATGGTCACCG |
| | | | TCTCCTCAGCCTCCACCAAGGGCCCATCGGTCTTCCCCCTGGCACCCTCCTC |
| | | | CAAGAGCACCTCTGGGGGCACAGCGGCCCTGGGCTGCCTGGTCAAGGACTA |
| | | | CTTCCCCGAACCGGTGACGGTGTCGTGGAACTCAGGCGCCCTGACCAGCGG |
| | | | CGTGCACACCTTCCCGGCTGTCCTACAGTCCTCAGGACTCTACTCCCTCAGC |
| | | | AGCGTGGTGACCGTGCCCTCCAGCAGCTTGGGCACCCAGACCTACATCTGC |
| | | | AACGTGAATCACAAGCCCAGCAACACCAAGGTGGACAAGAGAGTG |
| | VL | 47 | GCCTCTGCCCTGACTCAGCCTGCCTCCGTGTCTGGGTCTCCTGGACAGTCGA |
| | | | TCACCATCTCCTGCACTGGAACCAGCAGTGACGTTGGTGATTATAACTATGT |
| | | | CTCCTGGTACCAACAGCACCCAGGCAAAGCCCCCAAACTCATGATTTATGA |
| | | | TGTCAGTAATCGGCCCTCAGGGGTTTCTAATCGCTTCTCTGGCTCCAAGTCT |
| | | | GGCAACACGGCCTCCCTGACCATCTCTGGGCTCCAGGCTGAGGACGAGGCT |
| | | | GATTATTACTGCAGCTCATATACAAGCAGCAGCACTCTTGTATTCGGCGGA |
| | | | GGGACCAAGCTGACCGTCCTAGGTCAGCCCAAGGCTGCCCCCTCGGTCACT |
| | | | CTGTTCCCGCCCTCCTCTGAGGAGCTTCAAGCCAACAAGGCCACACTGGTG |
| | | | TGTCTCATAAGTGACTTCTACCCGGGAGCCGTGACAGTGGCCTGGAAGGCA |
| | | | GATAGCAGCCCCGTCAAGGCGGGAGTGGAGACCACCACACCCTCCAAACA |
| | | | AAGCAACAACAAGTACGCGGCCAGCAGCTATCTGAGCCTGACGCCTGAGC |
| | | | AGTGGAAGTCCCACAGAAGCTACAGCTGCCAGGTCACGCATGAAGGGAGC |
| | | | ACCGTGGAGAAGACAGTGGCCCCTACAGAATGTTCA |
| TMEL1003 | VH | 43 | GCCGTGCAGCTGGTGCAGTCCGGAGCAGAGGTGAAAAAGCCCGGGGAGTC |
| | | | TCTGAGGATCTCCTGTAAGGGTTCTGGATACAGCTTTACCAGCTACTGGATC |
| | | | AGCTGGGTGCGCCAGATGCCCGGGAAAGGCCTGGAGTGGATGGGGAGGAT |
| | | | TGATCCTAGTGACTCTTATACCAACTACAGCCCGTCCTTCCAAGGCCACGTC |
| | | | ACCATCTCAGCTGACAAGTCCATCAGCACTGCCTACCTACAGTGGAGCAGC |
| | | | CTGAAGACCTCGGACACCGCCATGTATTACTGTGCGAGACCGCTACAAACT |
| | | | TATAGTATAGCATCAGTAGGACACTGGGGCCAGGGAACCCTGGTCACCGTC |
| | | | TCCTCAGGGAGTGCATCCGCCCCAACCCTTTTCCCCCTCGTCTCCTGTGAGA |
| | | | ATTCCCCGTCGGATACGAGCAGCGTGGCCGTTGGCTGCCTCGCACAGGACT |
| | | | TCCTTCCCGACTCCATCACTTTCTCCTGGAAATACAAGAACAACTCTGACAT |
| | | | CAGCAGCACCCGGGGCTTCCCATCAGTCCTGAGAGGGGGCAAGTACGCAGC |
| | | | CACCTCACAGGTGCTGCTGCCTTCCAAGGACGTCATGCAGGGCACAGACGA |
| | | | ACACGTGGTGTGCAAAGTCCAGCACCCCAACGGCAACAAAGAAAAGAACG |
| | | | TGCCTCTTCCG |
| | VL | 48 | GCCTCTGCCCTGACTCAGCCTGCCTCCGTGTCTGGGTCTCCTGGACAGTCGA |
| | | | TCACCATCTCCTGCACTGGAACCAGCAGTGATGTTGGGAGTTATAACCTTGT |
| | | | CTCCTGGTACCAACAGCACCCAGGCAAAGCCCCCAAACTCATGATTTATGA |
| | | | GGGCAGTAAGCGGCCCTCAGGGGTTTCTAATCGCTTCTCTGGCTCCAAGTCT |
| | | | GGCAACACGGCCTCCCTGACAATCTCTGGGCTCCAGGCTGAGGACGAGGCT |
| | | | GATTATTACTGCTGCTCATATGCAGGTAGTAGCACTTTCGCGGTATTCGGCG |
| | | | GAGGGACCAAGCTGACCGTCCTAGGTCAGCCCAAGGCTGCCCCCTCGGTCA |
| | | | CTCTGTTCCCGCCCTCCTCTGAGGAGCTTCAAGCCAACAAGGCCACACTGGT |
| | | | GTGTCTCATAAGTGACTTCTACCCGGGAGCCGTGACAGTGGCCTGGAAGGC |
| | | | AGATAGCAGCCCCGTCAAGGCGGGAGTGGAGACCACCACACCCTCCAAAC |
| | | | AAAGCAACAACAAGTACGCGGCCAGCAGCTACCTGAGCCTGACGCCTGAG |
| | | | CAGTGGAAGTCCCACAGAAGCTACAGCTGCCAGGTCACGCATGAAGGGAG |
| | | | CACCGTGGAGAAGACAGTGGCCCCTACAGAATGTTCA |
| TMEL1004 | VH | 44 | CCCAGATCACCTTGAAGGAGTCTGGTCCGACGCTGGTGAAGCCCACACAGA |
| | | | CCCTCACGCTGACCTGCACCTTCTCTGGGTTCTCACTCAACACTCCTGGAGT |
| | | | GGGTGTGGGCTGGATCCGTCAGCCCCCAGGAAAGGCCCTGGAATGGCTTGC |
| | | | ACTCATTTATTGGGATGATGATAAGCGCTACAGGCCATCTCTGGAGAGCAG |
| | | | GCTCACCATCACCAAGGACACCTCCAAAAACCACGTTGTCCTTACGATGAC |
| | | | CAACATGGACCCTGTGGACACAGCCACATATTTTTGTGCACACAAGAACCT |
| | | | TCAGTATTCGGAATGGTTCGACCCCTGGGGCCAGGGCACCCTGGTCATTGT |
| | | | CTCCTCAGCCTCCACCAAGGGCCCATCGGTCTTCCCCCTGGCGCCCTGCTCC |
| | | | AGGAGCACCTCCGAGAGCACAGCGGCCCTGGGCTGCCTGGTCAAGGACTAC |
| | | | TTCCCCGAACCGGTGACGGTGTCGTGGAACTCAGGCGCCCTGACCAGCGGC |
| | | | GTGCACACCTTCCCGGCTGTCCTACAGTCCTCAGGACTCTACTCCCTCAGCA |
| | | | GCGTGGTGACCGTGCCCTCCAGCAGCTTGGGCACGAAGACCTACACCTGCA |
| | | | ATGTAGATCACAAGCCCAGCAACACCAAGGTGGACAAGAGAGTT |
| | VL | 49 | GAAATGGTGTTGACGCAGTCTCCAGCCACCCTGTCTTTGTCTCCAGGGGAA |
| | | | AGAGCCACCCTCTCCTGCAGGGCCAGTCAGAGTGTTAGCAGAAACTCCTTA |
| | | | GCCTGGTACCAGCAGAGACCTGGCCAGACTCCCAGGCTCCTCATCTATGGT |
| | | | GCCTCCAGCAGGGCCACTGGCATCCCAGACAGGTTCAGTGGCAGTGGGTCT |
| | | | GGGACAGACTTCACTCTCATCATCAGCAGACTGGAGCCTGAAGATTTTGCA |
| | | | GTGTATTTCTGTCTCCAGTATGATGAGTCACCGTACACTTTTGGCCAGGGGG |
| | | | CCAAGCTGGAGATCAAACGAACTGTGGCTGCACCATCTGTCTTCATCTTCCC |
| | | | GCCATCTGATGAGCAGTTGAAATCTGGAACTGCCTCTGTTGTGTGCCTGCTG |
| | | | AATAACTTCTATCCCAGAGAGGCCAAAGTACAGTGGAAGGTGGATAACGCC |
| | | | CTCCAATCGGGTAACTCCCAGGAGAGTGTCACAGAGCAGGACAGCAAGGA |
| | | | CAGCACCTACAGCCTCAGCAGCACCCTGACGCTGAGCAAAGCAGACTACGA |
| | | | GAAACACAAACTCTACGCCTGCGAAGTCACCCATCAGGGCCTGAGCTCGCC |
| | | | CGTCACAAAGAGCTTCAACAGGGGAGAGTGT |
| TMEL1005 | VH | 45 | CCCGTGCAGCTGCAGGAGTCGGGCCCAGGACTGGTGAAGCCTTCGGAGACC |
| | | | CTGTCCCTCACCTGCACTGTCTCTGGTGGCTCCATGAGCATTAGGAGTTCCT |
| | | | ACTGGGGCTGGATCCGCCAGTCACCAGGGAAGGGGCTGGAGTGGATTGGG |
| | | | CATATATTTTATAGTGGGAGCACCTACTACAACCCGTCCCTCCAGAGTCGA |
| | | | GTCACAATATTAGTAGACACGTCCAAGAACCAATTCTCCCTGAGGCTGAGC |
| | | | TCTGTGACCGCAGCGGACACGGCCGTGTATTACTGTGTGAGAAGTTTTGGC |
| | | | GTGGCTCGATGGGACTTCTGGGGCCAGGGAACCCTGGTCACCGTCTCCTCA |
| | | | GCCTCCACCAAGGGCCCATCGGTCTTCCCCCTGGCACCCTCCTCCAAGAGC |
| | | | ACCTCTGGGGGCACAGCGGCCCTGGGCTGCCTGGTCAAGGACTACTTCCCC |
| | | | GAACCGGTGACGGTGTCGTGGAACTCAGGCGCCCTGACCAGCGGCGTGCAC |
| | | | ACCTTCCCGGCTGTCCTACAGTCCTCAGGACTCTACTCCCTCAGCAGCGTGG |
| | | | TGACCGTGCCCTCCAGCAGCTTGGGCACCCAGACCTACATCTGCAACGTGA |
| | | | ATCACAAGCCCAGCAACACCAAGGTGGACAAGAAGGTG |
| | VL | 50 | GTGACATCCAGATGACCCAGTCTCCATCCTCCCTGTCTGCATCTGTAGGAGA |
| | | | CAGAGTCACCATCACTTGCCAGGCGAGTCAGAACATTAGGAATTATTTAAA |
| | | | TTGGTATCAGCAGAAACCAGGGAAAGCCCCTAAGCTCCTGATCTACGATGC |
| | | | ATCCAATTTGGAAACAGGGGTCCCATCAAGGTTCAGTGGAAGTGGATCTGG |
| | | | GACAGATTTTACTCTCACCATCAGCAGCCTGCAGCCTGAAGATATTGCAAC |
| | | | ATATTACTGTCAACAGTATGATAATCTCCTCCTATTCACTTTCGGCCCTGGG |
| | | | ACCACAGTTGATATCAAACGAACTGTGGCTGCACCATCTGTCTTCATCTTCC |
| | | | CGCCATCTGATGAGCAGTTGAAATCTGGAACTGCCTCTGTTGTGTGCCTGCT |
| | | | GAATAACTTCTATCCCAGAGAGGCCAAAGTACAGTGGAAGGTGGATAACG |
| | | | CCCTCCAATCGGGTAACTCCCAGGAGAGTGTCACAGAGCAGGACAGCAAG |
| | | | GACAGCACCTACAGCCTCAGCAGCACCCTGACGCTGAGCAAAGCAGACTAC |
| | | | GAGAAACACAAAGTCTACGCCTGCGAAGTCACCCATCAGGGCCTGAGCTCG |
| | | | CCCGTCACAAAGAGCTTCAACAGGGGAGAGTGT |

Table 4 below lists exemplary reconstructed nucleic acid consensus sequences of complementarity-determining region from a variable heavy chain (CDR-H3) and exemplary reconstructed nucleic acid consensus sequences of complementarity-determining region from a variable light chain (CDR-L3). The start and stop position of CDR3 on the corresponding isolated nucleic acid sequence is indicated.

| | Region | | | | Nucleic acid sequences of |
|---|---|---|---|---|---|
| | and | SEQ ID | Start | Stop | complementarity-determining |
| Name | Chain | No | position | Position | region 3 (CDR3) |
| TMEL1001 | CDR-H3 | 51 | 293 | 325 | GCGAGAGGTGGGGGTACCAGCTGG |
| | | | | | TCGCATTAC |
| | CDR-L3 | 56 | 274 | 303 | CAGTCTTATGATAGCAACAATCGTT |
| | | | | | GGGTG |
| TMEL1002 | CDR-H3 | 52 | 289 | 333 | GCGAAAGATGCATATGATAGTAGT |
| | | | | | GGCCCAGATGCTTTTGATATC |
| | CDR-L3 | 57 | 271 | 300 | AGCTCATATACAAGCAGCAGCACT |
| | | | | | CTTGTA |
| TMEL1003 | CDR-H3 | 53 | 289 | 330 | GCGAGACCGCTACAAACTTATAGT |
| | | | | | ATAGCATCAGTAGGACAC |
| | CDR-L3 | 58 | 271 | 303 | TGCTCATATGCAGGTAGTAGCACTT |
| | | | | | TCGCGGTA |
| TMEL1004 | CDR-H3 | 54 | 294 | 332 | GCACACAAGAACCTTCAGTATTCG |
| | | | | | GAATGGTTCGACCCC |
| | CDR-L3 | 59 | 268 | 294 | CTCCAGTATGATGAGTCACCGTACA |
| | | | | | CT |
| TMEL1005 | CDR-H3 | 55 | 292 | 324 | GTGAGAAGTTTTGGCGTGGCTCGAT |
| | | | | | GGGACTTC |
| | CDR-L3 | 60 | 267 | 296 | CAACAGTATGATAATCTCCTCCTAT |
| | | | | | TCACT |
| TMEL1001 | CDR-H2 | 61 | 155 | 178 | ATTAGTAGTAGTGGTAATTACATA |
| | CDR-L2 | 66 | 151 | 159 | GAGGATAAC |
| TMEL1002 | CDR-H2 | 62 | 151 | 174 | ATTAGTGGTAGTGGTGGTAGCACA |
| | CDR-L2 | 67 | 154 | 162 | GATGTCAGT |
| TMEL1003 | CDR-H2 | 63 | 151 | 174 | ATTGATCCTAGTGACTCTTATACC |
| | CDR-L2 | 68 | 154 | 162 | GAGGGCAGT |
| TMEL1004 | CDR-H2 | 64 | 159 | 179 | ATTTATTGGGATGATGATAAG |
| | CDR-L2 | 69 | 151 | 159 | GGTGCCTCC |
| TMEL1005 | CDR-H2 | 65 | 157 | 177 | ATATTTTATAGTGGGAGCACC |
| | CDR-L2 | 70 | 150 | 158 | GATGCATCC |
| TMEL1001 | CDR-H1 | 71 | 80 | 103 | GGATTCACCTTCAGGAGCTATAGC |
| | CDR-L1 | 76 | 76 | 99 | AGTGGCAGCATTGCCAGCAACTAT |
| TMEL1002 | CDR-H1 | 72 | 76 | 99 | GGATTCACCTTTAGCAGCTATGCC |
| | CDR-L1 | 77 | 76 | 102 | AGCAGTGACGTTGGTGATTATAACT |
| | | | | | AT |
| TMEL1003 | CDR-H1 | 73 | 76 | 99 | GGATACAGCTTTACCAGCTACTGG |
| | CDR-L1 | 78 | 76 | 102 | AGCAGTGATGTTGGGAGTTATAAC |
| | | | | | CTT |
| TMEL1004 | CDR-H1 | 74 | 78 | 107 | GGGTTCTCACTCAACACTCCTGGAG |
| | | | | | TGGGT |
| | CDR-L1 | 79 | 79 | 99 | CAGAGTGTTAGCAGAAACTCC |
| TMEL1005 | CDR-H1 | 75 | 76 | 105 | GGTGGCTCCATGAGCATTAGGAGTT |
| | | | | | CCTAC |
| | CDR-L1 | 80 | 81 | 98 | CAGAACATTAGGAATTAT |

Table 5 lists exemplary reconstructed germline amino acid consensus sequences of variable heavy chain (VH) and Exemplary reconstructed germline amino acid consensus sequences variable light chain (VL)

| | | SEQ | |
|---|---|---|---|
| Name | Chain | ID No | Amino acid sequences |
| TMEL1001 | VH | 81 | EVOLVESGGGLVKPGGSLRLSCAASGFTFSSYSMNWVRQA |
| | | | PGKGLEWVSSISSSSSYIYYADSVKGRFTISRDNAKNSLYLQ |
| | | | MNSLRAEDTAVYYCAREGYCSSTSCYATTLTTGAREPWSP |
| | | | SPQASTKGPSVFPLAPSSKSTSGGTAALGCLVKDYFPEPVT |
| | | | VSWNSGALTSGVHTFPAVLQSSGLYSLSSVVTVPSSSLGTQ |
| | | | TYICNVNHKPSNTKVDKKV |
| | VL | 86 | NFMLTQPHSVSESPGKTVTISCTRSSGSIASNYVQWYQQRP |
| | | | GSSPTTVIYEDNQRPSGVPDRFSGSIDSSSNSASLTISGLKTE |
| | | | DEADYYCQSYDSSNHWVFGGGTKLTVLAQGCPLGHSVPT |
| | | | LLGASSQQGHTGVSHKLLPGSRDSCLEGRQPRQGGGGDHH |
| | | | TLQTKQQQVRGQQLPEPDAAVEVPQKLQLPGHA- |
| | | | REHRGEDSCPYGMF |
| TMEL1002 | VH | 82 | EVQLLESGGGLVQPGGSLRLSCAASGFTFSSYAMSWVRQA |
| | | | PGKGLEWVSAISGSGGSTYYADSVKGRFTISRDNSKNTLYL |
| | | | QMNSLRAEDTAVYYCAKEYYYDSSGYYYCFYLGPRDNGH |
| | | | RLFRPPPRAHRSSPWHPPPRAPLGAQRPWAAWSRTTSPNR |
| | | | RCRGTQAPPAACTPSRLSYSPQDSTPSAAWPCPPAAWAPRP |
| | | | TSATITSPATPRWTRK |
| | VL | 87 | QSALTQPASVSGSPGQSITISCTGTSSDVGGYNYVSWYQQH |
| | | | PGKAPKLMIYEVSNRPSGVSNRFSGSKSGNTASLTISGLQA |
| | | | EDEADYYCSSYTSSSTLCGIRRRDQADRPRVSPRLPPRSLCS |
| | | | RPPLRSFKPTRPHWCVSVTSTREPQWLGKQIAAPSRREWRP |
| | | | PHPPNKATTSTRPAAIARLSSGSPTEATAARSRMKGAPWRR |
| | | | QWPLQNV |
| TMEL1003 | VH | 83 | EVQLVQSGAEVKKPGESLRISCKGSGYSFTSYWISWVRQM |
| | | | PGKGLEWMGRIDPSDSYTNYSPSFQGHVTISADKSISTAYL |
| | | | QWSSLKASDTAMYYCARGYSSSWYTTLTTGAREPWSPSP |
| | | | QGSASAPTLFPLVSCENSPSDTSSVAVGCLAQDFLPDSITLS |
| | | | WKYKNNSDISSTRGFPSVLRGGKYAATSQVLLPSKDVMQG |
| | | | TDEHVVCKVQHPNGNKEKNVPLP |
| | VL | 88 | QSALTQPASVSGSPGQSITISCTGTSSDVGSYNLVSWYQQH |
| | | | PGKAPKLMIYEGSKRPSGVSNRFSGSKSGNTASLTISGLQA |
| | | | EDEADYYCCSYAGSSTFCGIRRRDQADRPSPRLPPRSLCSH |
| | | | PPLRSFKPTRPHWCVSVTSTREPQLPGRQIAAPSRRGWRPP |
| | | | HPPNKATTSTRPAATARLSSGSPTKATAARSRMKGAPWRR |
| | | | QLPLRNV |
| TMEL1004 | VH | 84 | QITLKESGPTLVKPTQTLTLTCTFSGFSLSTSGVGVGWIRQP |
| | | | PGKALEWLALIYWDDDKRYSPSLKSRLTITKDTSKNQVVL |
| | | | TMTNMDPVDTATYYCAHRRYNRNHTTGSTPGAREPWSPS |
| | | | PQASTKGPSVFPLAPCSRSTSESTAALGCLVKDYFPEPVTVS |
| | | | WNSGALTSGVHTFPAVLQSSGLYSLSSVVTVPSSNFGTQTY |
| | | | TCNVDHKPSNTKVDKTV |
| | VL | 89 | EIVLTQSPGTLSLSPGERATLSCRASQSVSSSYLAWYQQKP |
| | | | GQAPRLLIYGASSRATGIPDRFSGSGSGTDFTLTISRLEPEDF |
| | | | AVYYCQQYGSSPPVHFWPGDQAGDQTELWLHHLSSSSRH |
| | | | LMSSNLELPLLCAC- |
| | | | ITSIPERPKYSGRWITPSNRVTPRRVSQSRTARTAPTASAAP |
| | | | RAKQTTRNTKSTPAKSPIRAARPSQRASTGES |
| TMEL1005 | VH | 85 | QLQLQESGPGLVKPSETLSLTCTVSGGSISSSSYYWGWIRQP |
| | | | PGKGLEWIGSIYYSGSTYYNPSLKSRVTISVDTSKNQFSLKL |
| | | | SSVTAADTAVYYCAREYYDFWSGYYTTTLTTGAREPWSPS |
| | | | PQASTKGPSVFPLAPSSKSTSGGTAALGCLVKDYFPEPVTV |
| | | | SWNSGALTSGVHTFPAVLQSSGLYSLSSVVTVPSSSLGTQT |
| | | | YICNVNHKPSNTKVDKKV |
| | VL | 90 | DIQMTQSPSSLSASVGDRVTITCQASQDISNYLNWYQQKPG |
| | | | KAPKLLIYDASNLETGVPSRFSGSGSGTDFTFTISSLQPEDIA |
| | | | TYYCQQYDNLPPFTFGPGTKVDIKPNCGCTICLHLPAIAVEI |
| | | | WNCLCCVPAELLSQRGQSTVEGGRPPIGLPGECHRAGQQG |
| | | | QHLQPQQHPDAEQSRLRETQSLRLRSHPSGPELARHKELQQ |
| | | | GRV |

Table 6 lists exemplary reconstructed germline nucleic acid consensus sequences of variable heavy chain (VH) and exemplary reconstructed germline nucleic acid consensus sequences of variable light chain (VL)

| | | SEQ | |
|---|---|---|---|
| Name | Chain | ID No | Nucleic acid sequences |
| TMEL1001 | VH | 91 | GAGGTGCAGCTGGTGGAGTCTGGGGGAGGCCTGGTCAAGCCTGGG |
| | | | GGGTCCCTGAGACTCTCCTGTGCAGCCTCTGGATTCACCTTCAGTA |
| | | | GCTATAGCATGAACTGGGTCCGCCAGGCTCCAGGGAAGGGGCTGG |
| | | | AGTGGGTCTCATCCATTAGTAGTAGTAGTAGTTACATATACTACGC |
| | | | AGACTCAGTGAAGGGCCGATTCACCATCTCCAGAGACAACGCCAA |
| | | | GAACTCACTGTATCTGCAAATGAACAGCCTGAGAGCCGAGGACAC |
| | | | GGCTGTGTATTACTGTGCGAGAGAAGGATATTGTAGTAGTACCAG |
| | | | CTGCTATGCCACTACTTTGACTACTGGGGCCAGGGAACCCTGGTCA |
| | | | CCGTCTCCTCAGGCCTCCACCAAGGGCCCATCGGTCTTCCCCCTGG |
| | | | CACCCTCCTCCAAGAGCACCTCTGGGGGCACAGCGGCCCTGGGCT |
| | | | GCCTGGTCAAGGACTACTTCCCCGAACCGGTGACGGTGTCGTGGA |
| | | | ACTCAGGCGCCCTGACCAGCGGCGTGCACACCTTCCCGGCTGTCCT |
| | | | ACAGTCCTCAGGACTCTACTCCCTCAGCAGCGTGGTGACCGTGCCC |
| | | | TCCAGCAGCTTGGGCACCCAGACCTACATCTGCAACGTGAATCAC |
| | | | AAGCCCAGCAACACCAAGGTGGACAAGAAAGTT |
| | VL | 96 | AATTTTATGCTGACTCAGCCCCACTCTGTGTCGGAGTCTCCGGGGA |
| | | | AGACGGTAACCATCTCCTGCACCCGCAGCAGTGGCAGCATTGCCA |
| | | | GCAACTATGTGCAGTGGTACCAGCAGCGCCCGGGCAGTTCCCCCA |
| | | | CCACTGTGATCTATGAGGATAACCAAAGACCCTCTGGGGTCCCTG |
| | | | ATCGGTTCTCTGGCTCCATCGACAGCTCCTCCAACTCTGCCTCCCTC |
| | | | ACCATCTCTGGACTGAAGACTGAGGACGAGGCTGACTACTACTGT |
| | | | CAGTCTTATGATAGCAGCAATCATTGGGTGTTCGGCGGAGGGACC |
| | | | AAGCTGACCGTCCTAGCCCAAGGCTGCCCCCTCGGTCACTCTGTTC |
| | | | CCACCCTCCTCTGAGGAGCTTCAAGCCAACAAGGCCACACTGGTG |
| | | | TGTCTCATAAGTGACTTCTACCCGGGAGCCGTGACAGTTGCCTGGA |
| | | | AGGCAGATAGCAGCCCCGTCAAGGCGGGGGTGGAGACCACCACA |
| | | | CCCTCCAAACAAAGCAACAACAAGTACGCGGCCAGCAGCTACCTG |
| | | | AGCCTGACGCCTGAGCAGTGGAAGTCCCACAAAAGCTACAGCTGC |
| | | | CAGGTCACGCATGAAGGGAGCACCGTGGAGAAGACAGTTGCCCCT |
| | | | ACGGAATGTTCA |
| TMEL1002 | VH | 92 | GAGGTGCAGCTGTTGGAGTCTGGGGGAGGCTTGGTACAGCCTGGG |
| | | | GGGTCCCTGAGACTCTCCTGTGCAGCCTCTGGATTCACCTTTAGCA |
| | | | GCTATGCCATGAGCTGGGTCCGCCAGGCTCCAGGGAAGGGGCTGG |
| | | | AGTGGGTCTCAGCTATTAGTGGTAGTGGTGGTAGCACATACTACGC |
| | | | AGACTCCGTGAAGGGCCGGTTCACCATCTCCAGAGACAATTCCAA |
| | | | GAACACGCTGTATCTGCAAATGAACAGCCTGAGAGCCGAGGACAC |
| | | | GGCCGTATATTACTGTGCGAAAGAGTATTACTATGATAGTAGTGGT |
| | | | TATTACTACTGATGCTTTTGATATCTGGGGCCAAGGGACAATGGTC |
| | | | ACCGTCTCTTCAGGCCTCCACCAAGGGCCCATCGGTCTTCCCCCTG |
| | | | GCACCCTCCTCCAAGAGCACCTCTGGGGGCACAGCGGCCCTGGGC |
| | | | TGCCTGGTCAAGGACTACTTCCCCGAACCGGTGACGGTGTCGTGG |
| | | | AACTCAGGCGCCCTGACCAGCGGCGTGCACACCTTCCCGGCTGTC |
| | | | CTACAGTCCTCAGGACTCTACTCCCTCAGCAGCGTGGTGACCGTGC |
| | | | CCTCCAGCAGCTTGGGCACCCAGACCTACATCTGCAACGTGAATC |
| | | | ACAAGCCCAGCAACACCAAGGTGGACAAGAAAGTT |
| | VL | 97 | CAGTCTGCCCTGACTCAGCCTGCCTCCGTGTCTGGGTCTCCTGGAC |
| | | | AGTCGATCACCATCTCCTGCACTGGAACCAGCAGTGACGTTGGTG |
| | | | GTTATAACTATGTCTCCTGGTACCAACAGCACCCAGGCAAAGCCCC |
| | | | CAAACTCATGATTTATGAGGTCAGTAATCGGCCCTCAGGGGTTTCT |
| | | | AATCGCTTCTCTGGCTCCAAGTCTGGCAACACGGCCTCCCTGACCA |
| | | | TCTCTGGGCTCCAGGCTGAGGACGAGGCTGATTATTACTGCAGCTC |
| | | | ATATACAAGCAGCAGCACTCTCTGTGGTATTCGGCGGAGGGACCA |
| | | | AGCTGACCGTCCTAGGGTCAGCCCAAGGCTGCCCCCTCGGTCACTC |
| | | | TGTTCCCGCCCTCCTCTGAGGAGCTTCAAGCCAACAAGGCCACACT |
| | | | GGTGTGTCTCATAAGTGACTTCTACCCGGGAGCCGTGACAGTGGCT |
| | | | TGGAAAGCAGATAGCAGCCCCGTCAAGGCGGGAGTGGAGACCAC |
| | | | CACACCCTCCAAACAAAGCAACAACAAGTACGCGGCCAGCAGCTA |
| | | | TCTGAGCCTGACGCCTGAGCAGTGGAAGTCCCACAGAAGCTACAG |
| | | | CTGCCAGGTCACGCATGAAGGGAGCACCGTGGAGAAGACAGTGGC |
| | | | CCCTACAGAATGTTCA |
| TMEL1003 | VH | 93 | GAAGTGCAGCTGGTGCAGTCCGGAGCAGAGGTGAAAAAGCCCGG |
| | | | GGAGTCTCTGAGGATCTCCTGTAAGGGTTCTGGATACAGCTTTACC |
| | | | AGCTACTGGATCAGCTGGGTGCGCCAGATGCCCGGGAAAGGCCTG |
| | | | GAGTGGATGGGGAGGATTGATCCTAGTGACTCTTATACCAACTAC |
| | | | AGCCCGTCCTTCCAAGGCCACGTCACCATCTCAGCTGACAAGTCCA |
| | | | TCAGCACTGCCTACCTGCAGTGGAGCAGCCTGAAGGCCTCGGACA |
| | | | CCGCCATGTATTACTGTGCGAGAGGGTATAGCAGCAGCTGGTACA |
| | | | CTACTTTGACTACTGGGGCCAGGGAACCCTGGTCACCGTCTCCTCA |
| | | | GGGGAGTGCATCCGCCCCAACCCTTTTCCCCCTCGTCTCCTGTGAG |
| | | | AATTCCCCGTCGGATACGAGCAGCGTGGCCGTTGGCTGCCTCGCAC |
| | | | AGGACTTCCTTCCCGACTCCATCACTTTGTCCTGGAAATACAAGAA |
| | | | CAACTCTGACATCAGCAGTACCCGGGGCTTCCCATCAGTCCTGAGA |
| | | | GGGGGCAAGTACGCAGCCACCTCACAGGTGCTGCTGCCTTCCAAG |
| | | | GACGTCATGCAGGGCACAGACGAACACGTGGTGTGCAAAGTCCAG |
| | | | CACCCCAACGGCAACAAAGAAAAGAACGTGCCTCTTCCA |
| | VL | 98 | CAGTCTGCCCTGACTCAGCCTGCCTCCGTGTCTGGGTCTCCTGGAC |
| | | | AGTCGATCACCATCTCCTGCACTGGAACCAGCAGTGATGTTGGGA |
| | | | GTTATAACCTTGTCTCCTGGTACCAACAGCACCCAGGCAAAGCCCC |
| | | | CAAACTCATGATTTATGAGGGCAGTAAGCGGCCCTCAGGGGTTTCT |
| | | | AATCGCTTCTCTGGCTCCAAGTCTGGCAACACGGCCTCCCTGACAA |
| | | | TCTCTGGGCTCCAGGCTGAGGACGAGGCTGATTATTACTGCTGCTC |
| | | | ATATGCAGGTAGTAGCACTTTCTGTGGTATTCGGCGGAGGGACCA |
| | | | AGCTGACCGTCCTAGCCCAAGGCTGCCCCCTCGGTCACTCTGTTCC |
| | | | CACCCTCCTCTGAGGAGCTTCAAGCCAACAAGGCCACACTGGTGT |
| | | | GTCTCATAAGTGACTTCTACCCGGGAGCCGTGACAGTTGCCTGGAA |
| | | | GGCAGATAGCAGCCCCGTCAAGGCGGGGGTGGAGACCACCACACC |
| | | | CTCCAAACAAAGCAACAACAAGTACGCGGCCAGCAGCTACCTGAG |
| | | | CCTGACGCCTGAGCAGTGGAAGTCCCACAAAAGCTACAGCTGCCA |
| | | | GGTCACGCATGAAGGGAGCACCGTGGAGAAGACAGTTGCCCCTAC |
| | | | GGAATGTTCA |
| TMEL1004 | VH | 94 | CAGATCACCTTGAAGGAGTCTGGTCCTACGCTGGTGAAACCCACA |
| | | | CAGACCCTCACGCTGACCTGCACCTTCTCTGGGTTCTCACTCAGCA |
| | | | CTAGTGGAGTGGGTGTGGGCTGGATCCGTCAGCCCCCAGGAAAGG |
| | | | CCCTGGAGTGGCTTGCACTCATTTATTGGGATGATGATAAGCGCTA |
| | | | CAGCCCATCTCTGAAGAGCAGGCTCACCATCACCAAGGACACCTC |
| | | | CAAAAACCAGGTGGTCCTTACAATGACCAACATGGACCCTGTGGA |
| | | | CACAGCCACATATTACTGTGCACACAGACGGTATAACCGGAACCA |
| | | | CACAACTGGTTCGACCCCTGGGGCCAGGGAACCCTGGTCACCGTC |
| | | | TCCTCAGGCCTCCACCAAGGGCCCATCGGTCTTCCCCCTGGCGCCC |
| | | | TGCTCCAGGAGCACCTCCGAGAGCACAGCCGCCCTGGGCTGCCTG |
| | | | GTCAAGGACTACTTCCCCGAACCGGTGACGGTGTCGTGGAACTCA |
| | | | GGCGCTCTGACCAGCGGCGTGCACACCTTCCCAGCTGTCCTACAGT |
| | | | CCTCAGGACTCTACTCCCTCAGCAGCGTGGTGACCGTGCCCTCCAG |
| | | | CAACTTCGGCACCCAGACCTACACCTGCAACGTAGATCACAAGCC |
| | | | CAGCAACACCAAGGTGGACAAGACAGTT |
| | VL | 99 | GAAATTGTGTTGACGCAGTCTCCAGGCACCCTGTCTTTGTCTCCAG |
| | | | GGGAAAGAGCCACCCTCTCCTGCAGGGCCAGTCAGAGTGTTAGCA |
| | | | GCAGCTACTTAGCCTGGTACCAGCAGAAACCTGGCCAGGCTCCCA |
| | | | GGCTCCTCATCTATGGTGCATCCAGCAGGGCCACTGGCATCCCAGA |
| | | | CAGGTTCAGTGGCAGTGGGTCTGGGACAGACTTCACTCTCACCATC |
| | | | AGCAGACTGGAGCCTGAAGATTTTGCAGTGTATTACTGTCAGCAGT |
| | | | ATGGTAGCTCACCTCCTGTACACTTTTGGCCAGGGGACCAAGCTGG |
| | | | AGATCAAACCGAACTGTGGCTGCACCATCTGTCTTCATCTTCCCGC |
| | | | CATCTGATGAGCAGTTGAAATCTGGAACTGCCTCTGTTGTGTGCCT |
| | | | GCTGAATAACTTCTATCCCAGAGAGGCCAAAGTACAGTGGAAGGT |
| | | | GGATAACGCCCTCCAATCGGGTAACTCCCAGGAGAGTGTCACAGA |
| | | | GCAGGACAGCAAGGACAGCACCTACAGCCTCAGCAGCACCCTGAC |
| | | | GCTGAGCAAAGCAGACTACGAGAAACACAAAGTCTACGCCTGCGA |
| | | | AGTCACCCATCAGGGCCTGAGCTCGCCCGTCACAAAGAGCTTCAA |
| | | | CAGGGGAGAGTGT |
| TMEL1005 | VH | 95 | CAGCTGCAGCTGCAGGAGTCGGGCCCAGGACTGGTGAAGCCTTCG |
| | | | GAGACCCTGTCCCTCACCTGCACTGTCTCTGGTGGCTCCATCAGCA |
| | | | GTAGTAGTTACTACTGGGGCTGGATCCGCCAGCCCCCAGGGAAGG |
| | | | GGCTGGAGTGGATTGGGAGTATCTATTATAGTGGGAGCACCTACT |
| | | | ACAACCCGTCCCTCAAGAGTCGAGTCACCATATCAGTAGACACGT |
| | | | CCAAGAACCAGTTCTCCCTGAAGCTGAGCTCTGTGACCGCCGCGG |
| | | | ACACGGCCGTGTATTACTGTGCGAGAGAGTATTACGATTTTTGGAG |
| | | | TGGTTATTATACCACTACTTTGACTACTGGGGCCAGGGAACCCTGG |
| | | | TCACCGTCTCCTCAGGCCTCCACCAAGGGCCCATCGGTCTTCCCCC |
| | | | TGGCACCCTCCTCCAAGAGCACCTCTGGGGGCACAGCGGCCCTGG |
| | | | GCTGCCTGGTCAAGGACTACTTCCCCGAACCGGTGACGGTGTCGTG |
| | | | GAACTCAGGCGCCCTGACCAGCGGCGTGCACACCTTCCCGGCTGT |
| | | | CCTACAGTCCTCAGGACTCTACTCCCTCAGCAGCGTGGTGACCGTG |
| | | | CCCTCCAGCAGCTTGGGCACCCAGACCTACATCTGCAACGTGAATC |
| | | | ACAAGCCCAGCAACACCAAGGTGGACAAGAAAGTT |
| | VL | 100 | GACATCCAGATGACCCAGTCTCCATCCTCCCTGTCTGCATCTGTAG |
| | | | GAGACAGAGTCACCATCACTTGCCAGGCGAGTCAGGACATTAGCA |
| | | | ACTATTTAAATTGGTATCAGCAGAAACCAGGGAAAGCCCCTAAGC |
| | | | TCCTGATCTACGATGCATCCAATTTGGAAACAGGGGTCCCATCAAG |
| | | | GTTCAGTGGAAGTGGATCTGGGACAGATTTTACTTTCACCATCAGC |
| | | | AGCCTGCAGCCTGAAGATATTGCAACATATTACTGTCAACAGTATG |
| | | | ATAATCTCCCTCCATTCACTTTCGGCCCTGGGACCAAAGTGGATAT |
| | | | CAAACCGAACTGTGGCTGCACCATCTGTCTTCATCTTCCCGCCATC |
| | | | TGATGAGCAGTTGAAATCTGGAACTGCCTCTGTTGTGTGCCTGCTG |
| | | | AATAACTTCTATCCCAGAGAGGCCAAAGTACAGTGGAAGGTGGAT |
| | | | AACGCCCTCCAATCGGGTAACTCCCAGGAGAGTGTCACAGAGCAG |
| | | | GACAGCAAGGACAGCACCTACAGCCTCAGCAGCACCCTGACGCTG |
| | | | AGCAAAGCAGACTACGAGAAACACAAAGTCTACGCCTGCGAAGTC |
| | | | ACCCATCAGGGCCTGAGCTCGCCCGTCACAAAGAGCTTCAACAGG |
| | | | GGAGAGTGT |

Table 7 lists exemplary heavy and light chain pairings

| | SEQ ID NOs | | | | | | | |
|---|---|---|---|---|---|---|---|---|
| | Variable region | | CDR 3 | | CDR 2 | | CDR 1 | |
| | Heavy | Light | Heavy | Light | Heavy | Light | Heavy | Light |
| | Chain | Chain | Chain | chain | Chain | chain | Chain | chain |
| Name | (VH) | (VL) | (CDR-H3) | (CDR-L3) | (CDR-H2) | (CDR-L2) | (CDR-H1) | (CDR-L1) |
| TMEL | SEQ ID | SEQ ID | SEQ ID | SEQ ID | SEQ ID | SEQ ID | SEQ ID | SEQ ID |
| 1001 | NO: 1 | NO: 6 | NO: 11 | NO: 16 | NO: 21 | NO: 26 | NO: 31 | NO: 36 |
| TMEL | SEQ ID | SEQ ID | SEQ ID | SEQ ID | SEQ ID | SEQ ID | SEQ ID | SEQ ID |
| 1002 | NO: 2 | NO: 7 | NO: 12 | NO: 17 | NO: 22 | NO: 27 | NO: 32 | NO: 37 |
| TMEL | SEQ ID | SEQ ID | SEQ ID | SEQ ID | SEQ ID | SEQ ID | SEQ ID | SEQ ID |
| 1003 | NO: 3 | NO: 8 | NO: 13 | NO: 18 | NO: 23 | NO: 28 | NO: 33 | NO: 38 |
| TMEL | SEQ ID | SEQ ID | SEQ ID | SEQ ID | SEQ ID | SEQ ID | SEQ ID | SEQ ID |
| 1004 | NO: 4 | NO: 9 | NO: 14 | NO: 19 | NO: 24 | NO: 29 | NO: 34 | NO: 39 |
| TMEL | SEQ ID | SEQ ID | SEQ ID | SEQ ID | SEQ ID | SEQ ID | SEQ ID | SEQ ID |
| 1005 | NO: 5 | NO: 10 | NO: 15 | NO: 20 | NO: 25 | NO: 30 | NO: 35 | NO: 40 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for generating a reconstructed consensus sequence coding for at least a portion of an immunoglobulin, comprising:
(a) obtaining ribonucleic acid sequence data for a plurality of biological samples obtained from subjects having a disease or disorder;
(b) processing the ribonucleic acid sequence data to identify a plurality of unique immunoglobulin clonotypes; and
(c) generating a reconstructed consensus sequence coding for at least a portion of the immunoglobulin based on the plurality of unique immunoglobulin clonotypes.

2. (canceled)

3. (canceled)

4. The method of claim 1, wherein processing the ribonucleic acid sequence data in (b) comprises filtering the ribonucleic acid sequence information.

5. The method of claim 4, wherein the filtering comprises removing non-functional clonotypes from further analysis.

6. The method of claim 1, wherein processing the ribonucleic acid sequence data in (b) comprises selecting a seed sequence from a predicted reference sequence and searching the ribonucleic acid sequence data for the seed sequence using a fuzzy pattern searching algorithm in order to identify at least one of the heavy D and light V-J junction for at least one of the plurality of sequences.

7. (canceled)

8. The method of claim 6, wherein processing the ribonucleic acid sequence data in (b) comprises selecting a new seed sequence from the predicted reference sequence and searching the ribonucleic acid sequence data for the new seed sequence

9. The method of claim 1, further comprising computing a diversity metric for at least a subset of the one or more biological samples, wherein the diversity metric is a measure of clonotype diversity.

10. The method of claim 9, wherein the diversity metric comprises an entropy index.

11.-41. (canceled)

42. A method for generating a reconstructed consensus sequence coding for at least a portion of an immunoglobulin, comprising:
(a) obtaining a plurality of biological samples from subjects having a disease or disorder;
(b) performing ribonucleic acid sequencing on the plurality of biological samples to obtain ribonucleic acid sequence data comprising a plurality of sequences;
(c) selecting a seed sequence from a predicted reference sequence and searching the ribonucleic acid sequence data for the seed sequence using a fuzzy pattern searching algorithm in order to identify at least one of the heavy D and light V-J junction for at least one of the plurality of sequences;
(d) filtering the ribonucleic acid sequence data to eliminate sequences that fail to achieve a threshold match ratio with the predicted reference sequence;
(e) selecting a new seed sequence from the predicted reference sequence and searching the ribonucleic acid sequence data for the new seed sequence;
(f) iteratively repeating step (e) until a threshold percentage of a J segment of the at least one of the plurality of sequences has been assembled;
(g) aligning and assembling a plurality of unique clonotypes based on the at least one of the plurality of sequences; and
(h) generating a reconstructed consensus sequence based on the aligned plurality of unique clonotypes.

43-48. (canceled)

49. A method of identifying protein dimers associated with a disease or disorder from mRNA sequencing data, the method comprising:
a. obtaining ribonucleic acid sequence data for a plurality of biological samples obtained from subjects having the disease or disorder;
b. processing the ribonucleic acid sequence data to identify a plurality of mRNA isoforms;
c. inferring at least one protein dimer from the plurality of unique mRNA isoforms, the at least one protein dimer comprising a first protein isoform and a second protein isoform inferred from the plurality of mRNA isoforms; and
d. generating a reconstructed consensus sequence coding for the at least one protein dimer based on the plurality of mRNA isoforms.

50. The method of claim 49, wherein processing the ribonucleic acid sequence data comprises aligning the ribonucleic acid sequencing data using a transcriptomic-referenced genomic aligner or a pseudo aligner.

51. The method of claim 51, further comprising discarding ribonucleic acid sequence data if the ribonucleic acid sequence data aligns to genomic loci that are at least 0.5 read lengths, one read length, or more than two read lengths away from a pair of loci known to code for two mRNA isoforms in a protein isomer.

52. The method of claim 49, wherein processing the ribonucleic acid sequence data comprises assembling the ribonucleic acid sequence data to identify the plurality of mRNA isoforms.

53. The method of claim 52, further comprising estimating the expression levels of the mRNA isoforms.

54. The method of claim 53, further comprising inferring, based on the expression levels of the mRNA isoforms, the probability of the protein dimer forming from the first mRNA isoform and the second mRNA isoform in vivo.

55.-70. (canceled)

71. A method for generating a reconstructed consensus sequence coding for at least a portion of a protein dimer, comprising:
a. obtaining ribonucleic acid sequence data for a plurality of biological samples obtained from subjects having a disease or disorder;
b. processing the ribonucleic acid sequence data to identify a plurality of unique protein isoforms; and
c. generating a reconstructed consensus sequence coding for at least a portion of the protein dimer based on the plurality of unique protein isoforms.

72. The method of claim 71, wherein the protein dimer is a human immunoglobulin.

73. The method of claim 72, wherein the human immunoglobulin is a candidate immunoglobulin for treating the disease or disorder.

74. The method of claim 71, wherein processing the ribonucleic acid sequence data in (b) comprises filtering the ribonucleic acid sequence information.

75.-110. (canceled)

111. A computer-implemented system for generating a reconstructed consensus sequence coding for at least a portion of a protein dimer, comprising at least one processor, an operating system configured to perform executable instructions, a memory, and instructions executable by the at least one processor to perform steps comprising:
a. obtaining ribonucleic acid sequence data for a plurality of biological samples obtained from subjects having a disease or disorder;
b. processing the ribonucleic acid sequence data to identify a plurality of unique protein isoforms; and
c. generating a reconstructed consensus sequence coding for at least a portion of the protein dimer based on the plurality of unique protein isoforms.

112-117. (canceled)
